SEQUENCE LISTING (353 sequences)

SEQ ID NO: 1 - DNA, 99 bp, Artificial Sequence
  Aptamer
taatacgactcactataggcgtagcctgatgaggcacaccacgtcaaaaatcattgtcaccacgaagccgaaacgtggtgaaagccacgtagctgcgcc

SEQ ID NO: 2 - DNA, 35 bp, Artificial Sequence
  signaling polynucleotide
gcacaccacgtcaaaaatcattgtcaccacgaagc

SEQ ID NO: 3 - DNA, 41 bp, Artificial Sequence
  Signaling polynucleotide
gcagcacaccacgtcaaaaatcattgtcaccacgaagctgc

SEQ ID NO: 4 - DNA, 45 bp, Artificial Sequence
  Signaling Polynucleotide
atgccgcacaccacgucaaaaaucauugucaccacgaagcggcat

SEQ ID NO: 5 - DNA, 99 bp, Artificial Sequence
  Aptamer
taatacgactcactataggcgtagcctgatgagtgcgcaacataagtctcttgaaagaccacgttcaacgaaacgtggtgaaagccacgtagctgcgcc

SEQ ID NO: 6 - DNA, 35 bp, Artificial Sequence
  Signaling Polynucleotide
tgcgcaacataagtctcttgaaagaccacgttcaa

SEQ ID NO: 7 - DNA, 49 bp, Artificial Sequence
  Signaling Polynucleotide
cctgatgagtgcgcaacataagtctcttgaaagaccacgttcaacgaaa

SEQ ID NO: 8 - DNA, 51 bp, Artificial Sequence
  Signaling Polynucleotide
uucctgatgagtgcgcaacataagtctcttgaaagaccacgttcaacgaaa

SEQ ID NO: 9 - DNA, 50 bp, Artificial Sequence
  Signaling Polynucleotide
uaactgatgagtgcgcaacataagtctcttgaaagaccacgttcaacgua

SEQ ID NO: 10 - DNA, 50 bp, Artificial Sequence
  Signaling Polynucleotide
uacgtgatgagtgcgcaacataagtctcttgaaagaccacgttcaacgua

SEQ ID NO: 11 - DNA, 49 bp, Artificial Sequence
  Signaling Polynucleotide
acatgatgagtgcgcaacataagtctcttgaaagaccacgttcaaaugu

SEQ ID NO: 12 - DNA, 99 bp, Artificial Sequence
  Aptamer
taatacgactcactataggcgtagcctgatgagcacccaccataccagaaatgttgacaccacgtggacgaaacgtggtgaaagccacgtagctgcgcc

SEQ ID NO: 13 - DNA, 35 bp, Artificial Sequence
  Signaling Polynucleotide
cacccaccataccagaaatgttgacaccacgtgga

SEQ ID NO: 14 - DNA, 54 bp, Artificial Sequence
  Signaling Polynucleotide
cctgatgagcacccaccataccagaaatgttgacaccacgtggacgaaacgtgg

SEQ ID NO: 15 - DNA, 43 bp, Artificial Sequence
  Signaling Polynucleotide
ugucacccaccataccagaaatgttgacaccacgtggacgaca

SEQ ID NO: 16 - DNA, 42 bp, Artificial Sequence
  Signaling Polynucleotide
uaucacccaccataccagaaatgttgacaccacgtggagaua

SEQ ID NO: 17 - DNA, 44 bp, Artificial Sequence
  Signaling Polynucleotide
ucgcacccaccataccagaaatgttgacaccacgtggaaaacga

SEQ ID NO: 18 - DNA, 44 bp, Artificial Sequence
  Signaling Polynucleotide
ucacacccaccataccagaaatgttgacaccacgtggaaaauga

SEQ ID NO: 19 - DNA, 48 bp, Artificial Sequence
  Signaling Polynucleotide
ucgucccacccaccataccagaaatgttgacaccacgtggaaaaacga

SEQ ID NO: 20 - DNA, 51 bp, Artificial Sequence
  Signaling Polynucleotide
cacccaccataccagaaatgttgacaccacgtggacgaaacgtggtgaaag

SEQ ID NO: 21 - DNA, 57 bp, Artificial Sequence
  Signaling Polynucleotide
cuucacccaccataccagaaatgttgacaccacgtggacgaaacgtggtgaaagaag

SEQ ID NO: 22 - DNA, 55 bp, Artificial Sequence
  Signaling Polynucleotide
uucacccaccataccagaaatgttgacaccacgtggacgaaacgtggtgaaagaa

SEQ ID NO: 23 - DNA, 53 bp, Artificial Sequence
  Signaling Polynucleotide
uacacccaccataccagaaatgttgacaccacgtggacgaaacgtggtgugua

SEQ ID NO: 24 - DNA, 54 bp, Artificial Sequence
  Signaling Polynucleotide
uacacccaccataccagaaatgttgacaccacgtggacgaaacgtggtgaugua

SEQ ID NO: 25 - DNA, 51 bp, Artificial Sequence
  Signaling Polynucleotide
uacacccaccataccagaaatgttgacaccacgtggacgaaacgtgcagua

SEQ ID NO: 26 - DNA, 99 bp, Artificial Sequence
  Aptamer
taatacgactcactataggcgtagcctgatgagtgcacaatgtaattatcaaaatacaccacgttggccgaaacgtggtgaaagccacgtagctgcgcc

SEQ ID NO: 27 - DNA, 35 bp, Artificial Sequence
  Signaling Polynucleotide
tgcacaatgtaattatcaaaatacaccacgttggc

SEQ ID NO: 28 - DNA, 61 bp, Artificial Sequence
  Signaling Polynucleotide
cctgatgagtgcacaatgtaattatcaaaatacaccacgttggccgaaacgtggtgaaagg

SEQ ID NO: 29 - DNA, 68 bp, Artificial Sequence
  Signaling Polynucleotide
ccaaacctgatgagtgcacaatgtaattatcaaaatacaccacgttggccgaaacgtggtgaccaagg

SEQ ID NO: 30 - DNA, 70 bp, Artificial Sequence
  Signaling Polynucleotide
ccaaauutgatgagtgcacaatgtaattatcaaaatacaccacgttggccgaaacgtggtgaaaccaugg

SEQ ID NO: 31 - DNA, 70 bp, Artificial Sequence
  Signaling Polynucleotide
uauaauutgatgagtgcacaatgtaattatcaaaatacaccacgttggccgaaacgtggtgaaaucucua

SEQ ID NO: 32 - DNA, 70 bp, Artificial Sequence
  Signaling Polynucleotide
uaugguutgatgagtgcacaatgtaattatcaaaatacaccacgttggccgaaacgtggtgaaaucaaua

SEQ ID NO: 33 - DNA, 68 bp, Artificial Sequence
  Signaling Polynucleotide
uguutgatgagtgcacaatgtaattatcaaaatacaccacgttggccgaaacgtggtgaaaucaaaca

SEQ ID NO: 34 - DNA, 99 bp, Artificial Sequence
  Aptamer
taatacgactcactataggcgtagcctgatgagccacatcgtgcaatgcccgaaacataccacgtagacgaaacgtggtgaaagccacgtagctgcgcc

SEQ ID NO: 35 - DNA, 35 bp, Artificial Sequence
  Signaling Polynucleotide
ccacatcgtgcaatgcccgaaacataccacgtaga

SEQ ID NO: 36 - DNA, 45 bp, Artificial Sequence
  Signaling Polynucleotide
ctgagccacatcgtgcaatgcccgaaacataccacgtagacucag

SEQ ID NO: 37 - DNA, 39 bp, Artificial Sequence
  Signaling Polynucleotide
gccacatcgtgcaatgcccgaaacataccacgtagaggc

SEQ ID NO: 38 - DNA, 35 bp, Artificial Sequence; identical to SEQ ID NO: 35
  Signaling Polynucleotide

SEQ ID NO: 39 - DNA, 49 bp, Artificial Sequence
  Signaling Polynucleotide
guccaaaccacatcgtgcaatgcccgaaacataccacgtagauggaaac

SEQ ID NO: 40 - DNA, 46 bp, Artificial Sequence
  Signaling Polynucleotide
cgcaaaccacatcgtgcaatgcccgaaacataccacgtagaaagcg

SEQ ID NO: 41 - DNA, 40 bp, Artificial Sequence
  Signaling Polynucleotide
ccacatcgtgcaatgcccgaaacataccacgtagaugugg

SEQ ID NO: 42 - DNA, 40 bp, Artificial Sequence
  Signaling Polynucleotide
ccacatcgtgcaatgcccgaaacataccacgtagacaagg

SEQ ID NO: 43 - DNA, 99 bp, Artificial Sequence
  Aptamer
taatacgactcactataggcgtagcctgatgagctatgcagtgatgattaaagataccaccacgtgagcgaaacgtggtgaaagccacgtagctgcgcc

SEQ ID NO: 44 - DNA, 35 bp, Artificial Sequence
  Signaling Polynucleotide
ctatgcagtgatgattaaagataccaccacgtgag

SEQ ID NO: 45 - DNA, 54 bp, Artificial Sequence
  Signaling Polynucleotide
gatgagctatgcagtgatgattaaagataccaccacgtgagcgaaacgtggtga

SEQ ID NO: 46 - DNA, 53 bp, Artificial Sequence
  Signaling Polynucleotide
gctatgcagtgatgattaaagataccaccacgtgagcgaaacgtggtgaaagc

SEQ ID NO: 47 - DNA, 53 bp, Artificial Sequence
  Signaling Polynucleotide
actatgcagtgatgattaaagataccaccacgtgagcgaaacgtggtgaaagu

SEQ ID NO: 48 - DNA, 53 bp, Artificial Sequence
  Signaling Polynucleotide
uactatgcagtgatgattaaagataccaccacgtgagcgaaacgtggtgagua

SEQ ID NO: 49 - DNA, 51 bp, Artificial Sequence
  Signaling Polynucleotide
gctatgcagtgatgattaaagataccaccacgtgagcgaaacgtggtccgc

SEQ ID NO: 50 - DNA, 62 bp, Artificial Sequence
  Signaling Polynucleotide
gguugatgagctatgcagtgatgattaaagataccaccacgtgagcgaaacgtggtgaaacc

SEQ ID NO: 51 - DNA, 60 bp, Artificial Sequence
  Signaling Polynucleotide
uuggatgagctatgcagtgatgattaaagataccaccacgtgagcgaaacgtggtgacaa

SEQ ID NO: 52 - DNA, 62 bp, Artificial Sequence
  Signaling Polynucleotide
ucggatgagctatgcagtgatgattaaagataccaccacgtgagcgaaacgtggtgaaacga

SEQ ID NO: 53 - DNA, 62 bp, Artificial Sequence
  Signaling Polynucleotide
augatgagctatgcagtgatgattaaagataccaccacgtgagcgaaacgtggtgaaaccau

SEQ ID NO: 54 - DNA, 59 bp, Artificial Sequence
  Signaling Polynucleotide
auggagctatgcagtgatgattaaagataccaccacgtgagcgaaacgtggtgaaacau

SEQ ID NO: 55 - DNA, 57 bp, Artificial Sequence
  Signaling Polynucleotide
guaaactatgcagtgatgattaaagataccaccacgtgagcgaaacgtggtgaaugc

SEQ ID NO: 56 - DNA, 88 bp, Artificial Sequence
  Aptamer
taatacgactcactataggcgtagcctgatgagcaaatagttacaaacaccacgtagcgaaacgtggtgaaagccacgtagctgcgcc

SEQ ID NO: 57 - DNA, 24 bp, Artificial Sequence
  Signaling Polynucleotide
caaatagttacaaacaccacgtag

SEQ ID NO: 58 - DNA, 32 bp, Artificial Sequence
  Signaling Polynucleotide
uucgcaaatagttacaaacaccacgtagcgaa

SEQ ID NO: 59 - DNA, 32 bp, Artificial Sequence
  Signaling Polynucleotide
aucgcaaatagttacaaacaccacgtagcgau

SEQ ID NO: 60 - DNA, 89 bp, Artificial Sequence
  Aptamer
taatacgactcactataggcgtagcctgatgagcccaactgtacagtacaccacgtagcgaaacgtggtgaaagccacgtagctgcgcc

SEQ ID NO: 61 - DNA, 25 bp, Artificial Sequence
  Signaling Polynucleotide
cccaactgtacagtacaccacgtag

SEQ ID NO: 62 - DNA, 46 bp, Artificial Sequence
  Signaling Polynucleotide
gagcccaactgtacagtacaccacgtagcgaaacgtggtgaaacuc

SEQ ID NO: 63 - DNA, 51 bp, Artificial Sequence
  Signaling Polynucleotide
uaucccaactgtacagtacaccacgtagcgaaacgtggtgaaaggaagaua

SEQ ID NO: 64 - DNA, 89 bp, Artificial Sequence
  Aptamer
taatacgactcactataggcgtagcctgatgagcacacacacattccaccacgtcacgcgaaacgtggtgaaagccacgtagctgcgcc

SEQ ID NO: 65 - DNA, 25 bp, Artificial Sequence
  Signaling Polynucleotide
cacacacacattccaccacgtcacg

SEQ ID NO: 66 - DNA, 33 bp, Artificial Sequence
  Signaling Polynucleotide
ctgatgagcacacacacattccaccacgtcacg

SEQ ID NO: 67 - DNA, 34 bp, Artificial Sequence
  Signaling Polynucleotide
gaugatgagcacacacacattccaccacgtcacg

SEQ ID NO: 68 - DNA, 36 bp, Artificial Sequence
  Signaling Polynucleotide
ugaugatgagcacacacacattccaccacgtcacga

SEQ ID NO: 69 - DNA, 39 bp, Artificial Sequence
  Signaling Polynucleotide
uuauugatgagcacacacacattccaccacgtcacguaa

SEQ ID NO: 70 - DNA, 89 bp, Artificial Sequence
  Aptamer
taatacgactcactataggcgtagcctgatgagcacacgttaccacaccacgttgacgcgaaacgtggtgaaagccacgtagctgcgcc

SEQ ID NO: 71 - DNA, 25 bp, Artificial Sequence
  Signaling Polynucleotide
cacacgttaccacaccacgttgacg

SEQ ID NO: 72 - DNA, 46 bp, Artificial Sequence
  Signaling Polynucleotide
auctgatgagcacacgttaccacaccacgttgacgcgaaacgagau

SEQ ID NO: 73 - DNA, 38 bp, Artificial Sequence
  Signaling Polynucleotide
tauugagcacacgttaccacaccacgttgacgcgaaua

SEQ ID NO: 74 - DNA, 40 bp, Artificial Sequence
  Signaling Polynucleotide
tgauugagcacacgttaccacaccacgttgacgcgaauca

SEQ ID NO: 75 - DNA, 35 bp, Artificial Sequence
  Signaling Polynucleotide
guugagcacacgttaccacaccacgttgacgcgaa

SEQ ID NO: 76 - DNA, 35 bp, Artificial Sequence
  Signaling Polynucleotide
auugagcacacgttaccacaccacgttgacgcgau

SEQ ID NO: 77 - DNA, 89 bp, Artificial Sequence
  Aptamer
taatacgactcactataggcgtagcctgatgagcgtgcccgaaacacacaccacgatgcgaaacgtggtgaaagccacgtagctgcgcc

SEQ ID NO: 78 - DNA, 25 bp, Artificial Sequence
  Signaling Polynucleotide
cgtgcccgaaacacacaccacgatg

SEQ ID NO: 79 - DNA, 41 bp, Artificial Sequence
  Signaling Polynucleotide
uugagcgtgcccgaaacacacaccacgatgcgaaacgucaa

SEQ ID NO: 80 - DNA, 40 bp, Artificial Sequence
  Signaling Polynucleotide
uuagcgtgcccgaaacacacaccacgatgcgaaacgcuaa

SEQ ID NO: 81 - DNA, 40 bp, Artificial Sequence
  Signaling Polynucleotide
gauagacgtgcccgaaacacacaccacgatgcgaaauguc

SEQ ID NO: 82 - DNA, 42 bp, Artificial Sequence
  Signaling Polynucleotide
ugauagacgtgcccgaaacacacaccacgatgcgaaauguca

SEQ ID NO: 83 - DNA, 89 bp, Artificial Sequence
  Aptamer
taatacgactcactataggcgtagcctgatgagctcaccacataccatgtaccacgtgcgaaacgtggtgaaagccacgtagctgcgcc

SEQ ID NO: 84 - DNA, 25 bp, Artificial Sequence
  Signaling Polynucleotide
ctcaccacataccatgtaccacgtg

SEQ ID NO: 85 - DNA, 50 bp, Artificial Sequence
  Signaling Polynucleotide
cctgatgagctcaccacataccatgtaccacgtgcgaaacgtggtgaagc

SEQ ID NO: 86 - DNA, 35 bp, Artificial Sequence
  Signaling Polynucleotide
gagctcaccacataccatgtaccacgtgcgaauga

SEQ ID NO: 87 - DNA, 32 bp, Artificial Sequence
  Signaling Polynucleotide
ctcaccacataccatgtaccacgtgcgaauga

SEQ ID NO: 88 - DNA, 53 bp, Artificial Sequence
  Signaling Polynucleotide
gcucctgatgagctcaccacataccatgtaccacgtgcgaaacgtggtgaagc

SEQ ID NO: 89 - DNA, 42 bp, Artificial Sequence
  Signaling Polynucleotide
caugatgagctcaccacataccatgtaccacgtgcgaacaug

SEQ ID NO: 90 - DNA, 41 bp, Artificial Sequence
  Signaling Polynucleotide
ucauacgagctcaccacataccatgtaccacgtgcgaauga

SEQ ID NO: 91 - DNA, 41 bp, Artificial Sequence; identical to SEQ ID NO: 90
  Signaling Polynucleotide

SEQ ID NO: 92 - DNA, 53 bp, Artificial Sequence; identical to SEQ ID NO: 88
  Signaling Polynucleotide

SEQ ID NO: 93 - DNA, 50 bp, Artificial Sequence; identical to SEQ ID NO: 85
  Signaling Polynucleotide

SEQ ID NO: 94 - DNA, 42 bp, Artificial Sequence; identical to SEQ ID NO: 89
  Signaling Polynucleotide

SEQ ID NO: 95 - DNA, 41 bp, Artificial Sequence; identical to SEQ ID NO: 90
  Signaling Polynucleotide

SEQ ID NO: 96 - DNA, 41 bp, Artificial Sequence; identical to SEQ ID NO: 90
  Signaling Polynucleotide

SEQ ID NO: 97 - DNA, 94 bp, Artificial Sequence
  Aptamer
taatacgactcactataggcgtagcctgatgagttcactggctgcacccaccaccgcgttccacgaaacgtggtgaaagccacgtagctgcgcc

SEQ ID NO: 98 - DNA, 30 bp, Artificial Sequence
  Signaling Polynucleotide
ttcactggctgcacccaccaccgcgttcca

SEQ ID NO: 99 - DNA, 55 bp, Artificial Sequence
  Signaling Polynucleotide
gatgagttcactggctgcacccaccaccgcgttccacgaaacgtggtgaaagcca

SEQ ID NO: 100 - DNA, 64 bp, Artificial Sequence
  Signaling Polynucleotide
cgucctgatgagttcactggctgcacccaccaccgcgttccacgaaacgtggtgaaagccaacg

SEQ ID NO: 101 - DNA, 66 bp, Artificial Sequence
  Signaling Polynucleotide
agcucctgatgagttcactggctgcacccaccaccgcgttccacgaaacgtggtgaaagccaagcu

SEQ ID NO: 102 - DNA, 59 bp, Artificial Sequence
  Signaling Polynucleotide
aucucctgatgagttcactggctgcacccaccaccgcgttccacgaaacgtggtgaaag

SEQ ID NO: 103 - DNA, 66 bp, Artificial Sequence
  Signaling Polynucleotide
gcuacctgatgagttcactggctgcacccaccaccgcgttccacgaaacgtggtgaaagccauagc

SEQ ID NO: 104 - DNA, 65 bp, Artificial Sequence
  Signaling Polynucleotide
ccacctgatgagttcactggctgcacccaccaccgcgttccacgaaacgtggtgaaagccaaagg

SEQ ID NO: 105 - DNA, 64 bp, Artificial Sequence
  Signaling Polynucleotide
uacctgatgagttcactggctgcacccaccaccgcgttccacgaaacgtggtgaaagccaggua

SEQ ID NO: 106 - DNA, 66 bp, Artificial Sequence
  Signaling Polynucleotide
auacctgatgagttcactggctgcacccaccaccgcgttccacgaaacgtggtgaaagccagguau

SEQ ID NO: 107 - DNA, 66 bp, Artificial Sequence
  Signaling Polynucleotide
cuacctgatgagttcactggctgcacccaccaccgcgttccacgaaacgtggtgaaagccaaauag

SEQ ID NO: 108 - DNA, 65 bp, Artificial Sequence
  Signaling Polynucleotide
uacctgatgagttcactggctgcacccaccaccgcgttccacgaaacgtggtgaaagccaaagua

SEQ ID NO: 109 - DNA, 94 bp, Artificial Sequence
  Aptamer
taatacgactcactataggcgtagcctgatgagcatccacggtgacgctaatcccacgttcgacgaaacgtggtgaaagccacgtagctgcgcc

SEQ ID NO: 110 - DNA, 30 bp, Artificial Sequence
  Signaling Polynucleotide
catccacggtgacgctaatcccacgttcga

SEQ ID NO: 111 - DNA, 46 bp, Artificial Sequence
  Signaling Polynucleotide
cctgatgagcatccacggtgacgctaatcccacgttcgacgaaagg

SEQ ID NO: 112 - DNA, 49 bp, Artificial Sequence
  Signaling Polynucleotide
agacctgatgagcatccacggtgacgctaatcccacgttcgacgaaucu

SEQ ID NO: 113 - DNA, 48 bp, Artificial Sequence
  Signaling Polynucleotide
agcctgatgagcatccacggtgacgctaatcccacgttcgacgaagcu

SEQ ID NO: 114 - DNA, 49 bp, Artificial Sequence
  Signaling Polynucleotide
uaucctgatgagcatccacggtgacgctaatcccacgttcgacggaaua

SEQ ID NO: 115 - DNA, 42 bp, Artificial Sequence
  Signaling Polynucleotide
gucgatgagcatccacggtgacgctaatcccacgttcgagac

SEQ ID NO: 116 - DNA, 40 bp, Artificial Sequence
  Signaling Polynucleotide
ucgatgagcatccacggtgacgctaatcccacgttcgaga

SEQ ID NO: 117 - DNA, 39 bp, Artificial Sequence
  Signaling Polynucleotide
ucgatgagcatccacggtgacgctaatcccacgttcgag

SEQ ID NO: 118 - DNA, 94 bp, Artificial Sequence
  Aptamer
taatacgactcactataggcgtagcctgatgagacaatgcagatgcgcccaccacggatcactcgaaacgtggtgaaagccacgtagctgcgcc

SEQ ID NO: 119 - DNA, 30 bp, Artificial Sequence
  Signaling Polynucleotide
acaatgcagatgcgcccaccacggatcact

SEQ ID NO: 120 - DNA, 58 bp, Artificial Sequence
  Signaling Polynucleotide
cctgatgagacaatgcagatgcgcccaccacggatcactcgaaacgtggtgaaagcca

SEQ ID NO: 121 - DNA, 33 bp, Artificial Sequence
  Signaling Polynucleotide
gagacaatgcagatgcgcccaccacggatcact

SEQ ID NO: 122 - DNA, 34 bp, Artificial Sequence
  Signaling Polynucleotide
gagacaatgcagatgcgcccaccacggatcactc

SEQ ID NO: 123 - DNA, 37 bp, Artificial Sequence
  Signaling Polynucleotide
gacacaatgcagatgcgcccaccacggatcactcguc

SEQ ID NO: 124 - DNA, 61 bp, Artificial Sequence
  Signaling Polynucleotide
cctgatgagacaatgcagatgcgcccaccacggatcactcgaaacgtggtgaaagccaagg

SEQ ID NO: 125 - DNA, 94 bp, Artificial Sequence
  Aptamer
taatacgactcactataggcgtagcctgatgagcaaccaagcacgctgcatcacgtttcatcgcgaaacgtggtgaaagccacgtagctgcgcc

SEQ ID NO: 126 - DNA, 30 bp, Artificial Sequence
  Signaling Polynucleotide
caaccaagcacgctgcatcacgtttcatcg

SEQ ID NO: 127 - DNA, 46 bp, Artificial Sequence
  Signaling Polynucleotide
gagcaaccaagcacgctgcatcacgtttcatcgcgaaacgtggcuc

SEQ ID NO: 128 - DNA, 46 bp, Artificial Sequence
  Signaling Polynucleotide
guucaaccaagcacgctgcatcacgtttcatcgcgaaacgtggaac

SEQ ID NO: 129 - DNA, 44 bp, Artificial Sequence
  Signaling Polynucleotide
uucaaccaagcacgctgcatcacgtttcatcgcgaaacgtugaa

SEQ ID NO: 130 - DNA, 37 bp, Artificial Sequence
  Signaling Polynucleotide
cgatgagcaaccaagcacgctgcatcacgtttcatcg

SEQ ID NO: 131 - DNA, 94 bp, Artificial Sequence
  Aptamer
taatacgactcactataggcgtagcctgatgagctcacagcccgaaacacatcgccacgttcacgaaacgtggtgaaagccacgtagctgcgcc

SEQ ID NO: 132 - DNA, 30 bp, Artificial Sequence
  Signaling Polynucleotide
ctcacagcccgaaacacatcgccacgttca

SEQ ID NO: 133 - DNA, 34 bp, Artificial Sequence
  Signaling Polynucleotide
tgagctcacagcccgaaacacatcgccacgttca

SEQ ID NO: 134 - DNA, 44 bp, Artificial Sequence
  Signaling Polynucleotide
cgaaatgagctcacagcccgaaacacatcgccacgttcaaaacg

SEQ ID NO: 135 - DNA, 54 bp, Artificial Sequence
  Signaling Polynucleotide
uaucgctcacagcccgaaacacatcgccacgttcacgaaacgtggtgaaagaua

SEQ ID NO: 136 - DNA, 104 bp, Artificial Sequence
  Aptamer
taatacgactcactataggcgtagcctgatgagctcaatactacgtcaattcacagatgatagacaccacggacgaaacgtggtgaaagccacgtagctgcgcc

SEQ ID NO: 137 - DNA, 40 bp, Artificial Sequence
  Signaling Polynucleotide
ctcaatactacgtcaattcacagatgatagacaccacgga

SEQ ID NO: 138 - DNA, 59 bp, Artificial Sequence
  Signaling Polynucleotide
gagctcaatactacgtcaattcacagatgatagacaccacggacgaaacgtggtgaaag

SEQ ID NO: 139 - DNA, 59 bp, Artificial Sequence
  Signaling Polynucleotide
gagctcaatactacgtcaattcacagatgatagacaccacggacgaaacgtggtgacuc

SEQ ID NO: 140 - DNA, 62 bp, Artificial Sequence
  Signaling Polynucleotide
guaagctcaatactacgtcaattcacagatgatagacaccacggacgaaacgtggtgacuac

SEQ ID NO: 141 - DNA, 62 bp, Artificial Sequence
  Signaling Polynucleotide
cuaagctcaatactacgtcaattcacagatgatagacaccacggacgaaacgtggtgacuag

SEQ ID NO: 142 - DNA, 60 bp, Artificial Sequence
  Signaling Polynucleotide
cuguctcaatactacgtcaattcacagatgatagacaccacggacgaaacgtggtgacag

SEQ ID NO: 143 - DNA, 60 bp, Artificial Sequence
  Signaling Polynucleotide
cugactcaatactacgtcaattcacagatgatagacaccacggacgaaacgtggtgacag

SEQ ID NO: 144 - DNA, 59 bp, Artificial Sequence
  Signaling Polynucleotide
cgactcaatactacgtcaattcacagatgatagacaccacggacgaaacgtggtguuug

SEQ ID NO: 145 - DNA, 62 bp, Artificial Sequence
  Signaling Polynucleotide
ucugactcaatactacgtcaattcacagatgatagacaccacggacgaaacgtggtgauaga

SEQ ID NO: 146 - DNA, 63 bp, Artificial Sequence
  Signaling Polynucleotide
aaaagctcaatactacgtcaattcacagatgatagacaccacggacgaaacgtggtgacuuau

SEQ ID NO: 147 - DNA, 63 bp, Artificial Sequence
  Signaling Polynucleotide
gaaagctcaatactacgtcaattcacagatgatagacaccacggacgaaacgtggtgacuuac

SEQ ID NO: 148 - DNA, 62 bp, Artificial Sequence
  Signaling Polynucleotide
cucagctcaatactacgtcaattcacagatgatagacaccacggacgaaacgtggtgacgag

SEQ ID NO: 149 - DNA, 104 bp, Artificial Sequence
  Aptamer
taatacgactcactataggcgtagcctgatgagtccaacaccacgtaacgtacactgcatgtgattggtgcaacgaaacgtggtgaaagccacgtagctgcgcc

SEQ ID NO: 150 - DNA, 40 bp, Artificial Sequence
  Signaling Polynucleotide
tccaacaccacgtaacgtacactgcatgtgattggtgcaa

SEQ ID NO: 151 - DNA, 59 bp, Artificial Sequence
  Signaling Polynucleotide
cctgatgagtccaacaccacgtaacgtacactgcatgtgattggtgcaacgaaacgtgg

SEQ ID NO: 152 - DNA, 65 bp, Artificial Sequence
  Signaling Polynucleotide
tagcctgatgagtccaacaccacgtaacgtacactgcatgtgattggtgcaacgaaacgtggtga

SEQ ID NO: 153 - DNA, 50 bp, Artificial Sequence
  Signaling Polynucleotide
gatgagtccaacaccacgtaacgtacactgcatgtgattggtgcaacgaa

SEQ ID NO: 154 - DNA, 58 bp, Artificial Sequence
  Signaling Polynucleotide
uuaagatgagtccaacaccacgtaacgtacactgcatgtgattggtgcaacgaauuaa

SEQ ID NO: 155 - DNA, 58 bp, Artificial Sequence
  Signaling Polynucleotide
ugaagatgagtccaacaccacgtaacgtacactgcatgtgattggtgcaacgaauuca

SEQ ID NO: 156 - DNA, 58 bp, Artificial Sequence
  Signaling Polynucleotide
ucacgatgagtccaacaccacgtaacgtacactgcatgtgattggtgcaacgaaguga

SEQ ID NO: 157 - DNA, 104 bp, Artificial Sequence
  Aptamer
taatacgactcactataggcgtagcctgatgagtggcgccgactgatcaactagacatcacgttagcattccgcgaaacgtggtgaaagccacgtagctgcgcc

SEQ ID NO: 158 - DNA, 40 bp, Artificial Sequence
  Signaling Polynucleotide
tggcgccgactgatcaactagacatcacgttagcattccg

SEQ ID NO: 159 - DNA, 59 bp, Artificial Sequence
  Signaling Polynucleotide
cctgatgagtggcgccgactgatcaactagacatcacgttagcattccgcgaaacgtgg

SEQ ID NO: 160 - DNA, 53 bp, Artificial Sequence
  Signaling Polynucleotide
gatgagtggcgccgactgatcaactagacatcacgttagcattccgcgaaacg

SEQ ID NO: 161 - DNA, 53 bp, Artificial Sequence
  Signaling Polynucleotide
ugtgagtggcgccgactgatcaactagacatcacgttagcattccgcgaaacg

SEQ ID NO: 162 - DNA, 59 bp, Artificial Sequence
  Signaling Polynucleotide
cctgatgagtggcgccgactgatcaactagacatcacgttagcattccgcgaaacgagg

SEQ ID NO: 163 - DNA, 65 bp, Artificial Sequence
  Signaling Polynucleotide
gatgagtggcgccgactgatcaactagacatcacgttagcattccgcgaaacgtggtgaaagcca

SEQ ID NO: 164 - DNA, 66 bp, Artificial Sequence
  Signaling Polynucleotide
catgagtggcgccgactgatcaactagacatcacgttagcattccgcgaaacgtggtgaaagcaug

SEQ ID NO: 165 - DNA, 66 bp, Artificial Sequence
  Signaling Polynucleotide
tagagtggcgccgactgatcaactagacatcacgttagcattccgcgaaacgtggtgaaagcucua

SEQ ID NO: 166 - DNA, 64 bp, Artificial Sequence
  Signaling Polynucleotide
agagtggcgccgactgatcaactagacatcacgttagcattccgcgaaacgtggtgaaagcuuu

SEQ ID NO: 167 - DNA, 104 bp, Artificial Sequence
  Aptamer
taatacgactcactataggcgtagcctgatgagccagcaaccaggttacctcccatcacgcttcgtctcaggacgaaacgtggtgaaagccacgtagctgcgcc

SEQ ID NO: 168 - DNA, 40 bp, Artificial Sequence
  Signaling Polynucleotide
ccagcaaccaggttacctcccatcacgcttcgtctcagga

SEQ ID NO: 169 - DNA, 56 bp, Artificial Sequence
  Signaling Polynucleotide
cctgatgagccagcaaccaggttacctcccatcacgcttcgtctcaggacgaaacg

SEQ ID NO: 170 - DNA, 53 bp, Artificial Sequence
  Signaling Polynucleotide
gatgagccagcaaccaggttacctcccatcacgcttcgtctcaggacgaaacg

SEQ ID NO: 171 - DNA, 50 bp, Artificial Sequence
  Signaling Polynucleotide
gatgagccagcaaccaggttacctcccatcacgcttcgtctcaggacgaa

SEQ ID NO: 172 - DNA, 56 bp, Artificial Sequence
  Signaling Polynucleotide
caagatgagccagcaaccaggttacctcccatcacgcttcgtctcaggacgaauug

SEQ ID NO: 173 - DNA, 56 bp, Artificial Sequence
  Signaling Polynucleotide
caagatgagccagcaaccaggttacctcccatcacgcttcgtctcaggacgauuug

SEQ ID NO: 174 - DNA, 59 bp, Artificial Sequence
  Signaling Polynucleotide
ccaagatgagccagcaaccaggttacctcccatcacgcttcgtctcaggacgacuuugg

SEQ ID NO: 175 - DNA, 59 bp, Artificial Sequence
  Signaling Polynucleotide
acaagatgagccagcaaccaggttacctcccatcacgcttcgtctcaggacgacuuugu

SEQ ID NO: 176 - DNA, 58 bp, Artificial Sequence
  Signaling Polynucleotide
acagatgagccagcaaccaggttacctcccatcacgcttcgtctcaggacgacuuugu

SEQ ID NO: 177 - DNA, 104 bp, Artificial Sequence
  Aptamer
taatacgactcactataggcgtagcctgatgagctgacaccacaaacgattatgaccacgttatcgtacatagcgaaacgtggtgaaagccacgtagctgcgcc

SEQ ID NO: 178 - DNA, 40 bp, Artificial Sequence
  Signaling Polynucleotide
ctgacaccacaaacgattatgaccacgttatcgtacatag

SEQ ID NO: 179 - DNA, 62 bp, Artificial Sequence
  Signaling Polynucleotide
taggcgtagcctgatgagctgacaccacaaacgattatgaccacgttatcgtacatagcgaa

SEQ ID NO: 180 - DNA, 62 bp, Artificial Sequence
  Signaling Polynucleotide
gagctgacaccacaaacgattatgaccacgttatcgtacatagcgaaacgtggtgaaagcca

SEQ ID NO: 181 - DNA, 64 bp, Artificial Sequence
  Signaling Polynucleotide
agagctgacaccacaaacgattatgaccacgttatcgtacatagcgaaacgtggtgaaagccau

SEQ ID NO: 182 - DNA, 65 bp, Artificial Sequence
  Signaling Polynucleotide
ugagctgacaccacaaacgattatgaccacgttatcgtacatagcgaaacgtggtgaaagccuca

SEQ ID NO: 183 - DNA, 67 bp, Artificial Sequence
  Signaling Polynucleotide
ugaagctgacaccacaaacgattatgaccacgttatcgtacatagcgaaacgtggtgaaagccguca

SEQ ID NO: 184 - DNA, 60 bp, Artificial Sequence
  Signaling Polynucleotide
ugaagctgacaccacaaacgattatgaccacgttatcgtacatagcgaaacgtggcguca

SEQ ID NO: 185 - DNA, 62 bp, Artificial Sequence
  Signaling Polynucleotide
augaagctgacaccacaaacgattatgaccacgttatcgtacatagcgaaacgtggcgucau

SEQ ID NO: 186 - DNA, 65 bp, Artificial Sequence
  Signaling Polynucleotide
guaactgacaccacaaacgattatgaccacgttatcgtacatagcgaaacgtggtgaaagccuac

SEQ ID NO: 187 - DNA, 67 bp, Artificial Sequence
  Signaling Polynucleotide
uguaagctgacaccacaaacgattatgaccacgttatcgtacatagcgaaacgtggtgaaagaggca

SEQ ID NO: 188 - DNA, 68 bp, Artificial Sequence
  Signaling Polynucleotide
uguaaagctgacaccacaaacgattatgaccacgttatcgtacatagcgaaacgtggtgaaagaggca

SEQ ID NO: 189 - DNA, 104 bp, Artificial Sequence
  Aptamer
taatacgactcactataggcgtagcctgatgagtaggtcaagtgcgctaaaacacaccgcgttagttcaccaacgaaacgtggtgaaagccacgtagctgcgcc

SEQ ID NO: 190 - DNA, 40 bp, Artificial Sequence
  Signaling Polynucleotide
taggtcaagtgcgctaaaacacaccgcgttagttcaccaa

SEQ ID NO: 191 - DNA, 62 bp, Artificial Sequence
  Signaling Polynucleotide
ataggtcaagtgcgctaaaacacaccgcgttagttcaccaacgaaacgtggtgaaagauaau

SEQ ID NO: 192 - DNA, 64 bp, Artificial Sequence
  Signaling Polynucleotide
gataggtcaagtgcgctaaaacacaccgcgttagttcaccaacgaaacgtggtgaaagauaauc

SEQ ID NO: 193 - DNA, 109 bp, Artificial Sequence
  Aptamer
taatacgactcactataggcgtagcctgatgaggccacctcactgtgttttgttgcacaacataatatgatgacgtgccgaaacgtggtgaaagccacgtagctgcgcc

SEQ ID NO: 194 - DNA, 46 bp, Artificial Sequence
  Signaling Polynucleotide
ggccacctcactgtgttttgttgcacaacataatatgatgacgtgc

SEQ ID NO: 195 - RNA, 52 nt, Artificial Sequence
  Signaling Polynucleotide
gcacgaggccaccucacuguguuuuguugcacaacauaauaugaugacgugc

SEQ ID NO: 196 - RNA, 53 nt, Artificial Sequence
  Signaling Polynucleotide
gcacguaggccaccucacuguguuuuguugcacaacauaauaugaugacgugc

SEQ ID NO: 197 - RNA, 54 nt, Artificial Sequence
  Signaling Polynucleotide
gcacguauggccaccucacuguguuuuguugcacaacauaauaugaugacgugc

SEQ ID NO: 198 - RNA, 53 nt, Artificial Sequence
  Signaling Polynucleotide
gcaccugggccaccucacuguguuuuguugcacaacauaauaugaugacgugc

SEQ ID NO: 199 - RNA, 50 nt, Artificial Sequence
  Signaling Polynucleotide
gcacggccaccucacuguguuuuguugcacaacauaauaugaugacgugc

SEQ ID NO: 200 - DNA, 52 bp, Artificial Sequence
  Signaling Polynucleotide
ttggccaccucacuguguuuuguugcacaacauaauaugaugacgugccgaa

SEQ ID NO: 201 - RNA, 51 nt, Artificial Sequence
  Signaling Polynucleotide
gccaccucacuguguuuuguugcacaacauaauaugaugacgugcgguggc

SEQ ID NO: 202 - RNA, 49 nt, Artificial Sequence
  Signaling Polynucleotide
gcacgccaccucacuguguuuuguugcacaacauaauaugaugacgugc

SEQ ID NO: 203 - DNA, 109 bp, Artificial Sequence
  Aptamer
taatacgactcactataggcgtagcctgatgaggcgttccccaccgttgcccacgcttaactggacaaagatgggccccgaaacgtggtgaaagccacgtagctgcgcc

SEQ ID NO: 204 - DNA, 45 bp, Artificial Sequence
  Signaling Polynucleotide
gcgttccccaccgttgcccacgcttaactggacaaagatgggccc

SEQ ID NO: 205 - DNA, 50 bp, Artificial Sequence
  Signaling Polynucleotide
gcgttccccaccgttgcccacgcttaactggacaaagatgggcccaacgc

SEQ ID NO: 206 - DNA, 52 bp, Artificial Sequence
  Signaling Polynucleotide
gggcccagcgttccccaccgttgcccacgcttaactggacaaagatgggccc

SEQ ID NO: 207 - DNA, 52 bp, Artificial Sequence
  Signaling Polynucleotide
accgcgttccccaccgttgcccacgcttaactggacaaagatgggcccagga

SEQ ID NO: 208 - DNA, 51 bp, Artificial Sequence
  Signaling Polynucleotide
ttgggcgttccccaccgttgcccacgcttaactggacaaagatgggcccga

SEQ ID NO: 209 - DNA, 52 bp, Artificial Sequence
  Signaling Polynucleotide
tagggcgttccccaccgttgcccacgcttaactggacaaagatgggcccgaa

SEQ ID NO: 210 - DNA, 48 bp, Artificial Sequence
  Signaling Polynucleotide
tggcgttccccaccgttgcccacgcttaactggacaaagatgggccca

SEQ ID NO: 211 - DNA, 50 bp, Artificial Sequence
  Signaling Polynucleotide
ugcgttccccaccgttgcccacgcttaactggacaaagatgggcccugug

SEQ ID NO: 212 - DNA, 53 bp, Artificial Sequence
  Signaling Polynucleotide
ttgggcgttccccaccgttgcccacgcttaactggacaaagatgggcccggaa

SEQ ID NO: 213 - DNA, 51 bp, Artificial Sequence
  Signaling Polynucleotide
ttcgcgttccccaccgttgcccacgcttaactggacaaagatgggccugag

SEQ ID NO: 214 - DNA, 109 bp, Artificial Sequence
  Aptamer
taatacgactcactataggcgtagcctgatgagtctgtgcacatcactcgacctctacggctgtattgatcctgcatacgaaacgtggtgaaagccacgtagctgcgcc

SEQ ID NO: 215 - DNA, 45 bp, Artificial Sequence
  Signaling Polynucleotide
tctgtgcacatcactcgacctctacggctgtattgatcctgcata

SEQ ID NO: 216 - DNA, 54 bp, Artificial Sequence
  Signaling Polynucleotide
tctgtgcacatcactcgacctctacggctgtattgatcctgcatagugcacaga

SEQ ID NO: 217 - DNA, 54 bp, Artificial Sequence
  Signaling Polynucleotide
ccuaugctctgtgcacatcactcgacctctacggctgtattgatcctgcatagg

SEQ ID NO: 218 - DNA, 53 bp, Artificial Sequence
  Signaling Polynucleotide
gcuagagtgcacatcactcgacctctacggctgtattgatcctgcataagagc

SEQ ID NO: 219 - DNA, 109 bp, Artificial Sequence
  Aptamer
taatacgactcactataggcgtagcctgatgagcgtccaacgttcgatcagaaccgcgttcaggctgatgattgtacgcgaaacgtggtgaaagccacgtagctgcgcc

SEQ ID NO: 220 - DNA, 45 bp, Artificial Sequence
  Signaling Polynucleotide
cgtccaacgttcgatcagaaccgcgttcaggctgatgattgtacg

SEQ ID NO: 221 - DNA, 52 bp, Artificial Sequence
  Signaling Polynucleotide
cgtccaacgttcgatcagaaccgcgttcaggctgatgattgtacguuccacg

SEQ ID NO: 222 - DNA, 55 bp, Artificial Sequence
  Signaling Polynucleotide
gcuuacgtccaacgttcgatcagaaccgcgttcaggctgatgattgtacguaagc

SEQ ID NO: 223 - DNA, 59 bp, Artificial Sequence
  Signaling Polynucleotide
tgatgagcgtccaacgttcgatcagaaccgcgttcaggctgatgattgtacgcgaaacg

SEQ ID NO: 224 - DNA, 59 bp, Artificial Sequence
  Signaling Polynucleotide
cgtgatgagcgtccaacgttcgatcagaaccgcgttcaggctgatgattgtacgcgacg

SEQ ID NO: 225 - DNA, 63 bp, Artificial Sequence
  Signaling Polynucleotide
caagtgatgagcgtccaacgttcgatcagaaccgcgttcaggctgatgattgtacgcgacttg

SEQ ID NO: 226 - DNA, 63 bp, Artificial Sequence
  Signaling Polynucleotide
auagtgatgagcgtccaacgttcgatcagaaccgcgttcaggctgatgattgtacgcgactat

SEQ ID NO: 227 - DNA, 109 bp, Artificial Sequence
  Aptamer
taatacgactcactataggcgtagcctgatgagcatcagtgcgttctgcctttgcaaccacacaacacaccgtatgagcgaaacgtggtgaaagccacgtagctgcgcc

SEQ ID NO: 228 - DNA, 45 bp, Artificial Sequence
  Signaling Polynucleotide
catcagtgcgttctgcctttgcaaccacacaacacaccgtatgag

SEQ ID NO: 229 - DNA, 51 bp, Artificial Sequence
  Signaling Polynucleotide
cucauacatcagtgcgttctgcctttgcaaccacacaacacaccgtatgag

SEQ ID NO: 230 - DNA, 55 bp, Artificial Sequence
  Signaling Polynucleotide
gcuuacatcagtgcgttctgcctttgcaaccacacaacacaccgtatgaguaagc

SEQ ID NO: 231 - DNA, 61 bp, Artificial Sequence
  Signaling Polynucleotide
cctgatgagcatcagtgcgttctgcctttgcaaccacacaacacaccgtatgagcgaaacg

SEQ ID NO: 232 - DNA, 52 bp, Artificial Sequence
  Signaling Polynucleotide
gcagcatcagtgcgttctgcctttgcaaccacacaacacaccgtatgagcgc

SEQ ID NO: 233 - DNA, 54 bp, Artificial Sequence
  Signaling Polynucleotide
ttgagcatcagtgcgttctgcctttgcaaccacacaacacaccgtatgagcgaa

SEQ ID NO: 234 - DNA, 54 bp, Artificial Sequence
  Signaling Polynucleotide
tgcagcatcagtgcgttctgcctttgcaaccacacaacacaccgtatgagcgcg

SEQ ID NO: 235 - DNA, 54 bp, Artificial Sequence
  Signaling Polynucleotide
cgcagcatcagtgcgttctgcctttgcaaccacacaacacaccgtatgagcgcg

SEQ ID NO: 236 - DNA, 109 bp, Artificial Sequence
  Aptamer
taatacgactcactataggcgtagcctgatgagccaactgtgcacactgttcgcttatcgagctgtgtacctccatagcgaaacgtggtgaaagccacgtagctgcgcc

SEQ ID NO: 237 - DNA, 45 bp, Artificial Sequence
  Signaling Polynucleotide
ccaactgtgcacactgttcgcttatcgagctgtgtacctccatag

SEQ ID NO: 238 - DNA, 64 bp, Artificial Sequence
  Signaling Polynucleotide
cctgatgagccaactgtgcacactgttcgcttatcgagctgtgtacctccatagcgaaacgtgg

SEQ ID NO: 239 - DNA, 70 bp, Artificial Sequence
  Signaling Polynucleotide
acucctgatgagccaactgtgcacactgttcgcttatcgagctgtgtacctccatagcgaaacgaggcat

SEQ ID NO: 240 - DNA, 71 bp, Artificial Sequence
  Signaling Polynucleotide
atgcctgatgagccaactgtgcacactgttcgcttatcgagctgtgtacctccatagcgaaacgtggtgaa

SEQ ID NO: 241 - DNA, 68 bp, Artificial Sequence
  Signaling Polynucleotide
aacctgatgagccaactgtgcacactgttcgcttatcgagctgtgtacctccatagcgaaacgtggca

SEQ ID NO: 242 - DNA, 65 bp, Artificial Sequence
  Signaling Polynucleotide
aactgatgagccaactgtgcacactgttcgcttatcgagctgtgtacctccatagcgaaacaguu

SEQ ID NO: 243 - DNA, 75 bp, Artificial Sequence
  Signaling Polynucleotide
catgcctgatgagccaactgtgcacactgttcgcttatcgagctgtgtacctccatagcgaaacgtggtgaagug

SEQ ID NO: 244 - DNA, 50 bp, Artificial Sequence
  Signaling Polynucleotide
ccaactgtgcacactgttcgcttatcgagctgtgtacctccatagguugg

SEQ ID NO: 245 - DNA, 51 bp, Artificial Sequence
  Signaling Polynucleotide
cuauggccaactgtgcacactgttcgcttatcgagctgtgtacctccatag

SEQ ID NO: 246 - DNA, 94 bp, Artificial Sequence
  Aptamer
taatacgactcactataggcgtagcctgatgagcttggtcacctttcctgacattaacacaggcgaaacgtggtgaaagccacgtagctgcgcc

SEQ ID NO: 247 - DNA, 30 bp, Artificial Sequence
  Signaling Polynucleotide
cttggtcacctttcctgacattaacacagg

SEQ ID NO: 248 - DNA, 35 bp, Artificial Sequence
  Signaling Polynucleotide
cttggtcacctttcctgacattaacacaggccaag

SEQ ID NO: 249 - DNA, 35 bp, Artificial Sequence
  Signaling Polynucleotide
ccugucttggtcacctttcctgacattaacacagg

SEQ ID NO: 250 - DNA, 94 bp, Artificial Sequence
  Aptamer
taatacgactcactataggcgtagcctgatgagttttcccgatacggctacgaattgcgacaacgaaacgtggtgaaagccacgtagctgcgcc

SEQ ID NO: 251 - DNA, 30 bp, Artificial Sequence
  Signaling Polynucleotide
ttttcccgatacggctacgaattgcgacaa

SEQ ID NO: 252 - DNA, 36 bp, Artificial Sequence
  Signaling Polynucleotide
ccttttcccgatacggctacgaattgcgacaaaagg

SEQ ID NO: 253 - DNA, 40 bp, Artificial Sequence
  Signaling Polynucleotide
gcuuattttcccgatacggctacgaattgcgacaauaagc

SEQ ID NO: 254 - DNA, 94 bp, Artificial Sequence
  Aptamer
taatacgactcactataggcgtagcctgatgaggcaccaattttaccgattttggtggacagccgaaacgtggtgaaagccacgtagctgcgcc

SEQ ID NO: 255 - DNA, 30 bp, Artificial Sequence
  Signaling Polynucleotide
gcaccaattttaccgattttggtggacagc

SEQ ID NO: 256 - DNA, 37 bp, Artificial Sequence
  Signaling Polynucleotide
gcuguccgcaccaattttaccgattttggtggacagc

SEQ ID NO: 257 - DNA, 37 bp, Artificial Sequence
  Signaling Polynucleotide
gcaccaattttaccgattttggtggacagcuuggugc

SEQ ID NO: 258 - DNA, 94 bp, Artificial Sequence
  Aptamer
taatacgactcactataggcgtagcctgatgagcgtacaacccaccaccgttgtccacaaatgcgaaacgtggtgaaagccacgtagctgcgcc

SEQ ID NO: 259 - DNA, 30 bp, Artificial Sequence
  Signaling Polynucleotide
cgtacaacccaccaccgttgtccacaaatg

SEQ ID NO: 260 - DNA, 36 bp, Artificial Sequence
  Signaling Polynucleotide
cauuugcgtacaacccaccaccgttgtccacaaatg

SEQ ID NO: 261 - DNA, 37 bp, Artificial Sequence
  Signaling Polynucleotide
cgtacaacccaccaccgttgtccacaaatguuguacg

SEQ ID NO: 262 - DNA, 94 bp, Artificial Sequence
  Aptamer
taatacgactcactataggcgtagcctgatgagtgcgtcaacggccgtcccgaaacgtgaatacgaaacgtggtgaaagccacgtagctgcgcc

SEQ ID NO: 263 - DNA, 30 bp, Artificial Sequence
  Signaling Polynucleotide
tgcgtcaacggccgtcccgaaacgtgaata

SEQ ID NO: 264 - DNA, 36 bp, Artificial Sequence
  Signaling Polynucleotide
uauucatgcgtcaacggccgtcccgaaacgtgaata

SEQ ID NO: 265 - DNA, 40 bp, Artificial Sequence
  Signaling Polynucleotide
gcuuatgcgtcaacggccgtcccgaaacgtgaatauaagc

SEQ ID NO: 266 - DNA, 94 bp, Artificial Sequence
  Aptamer
taatacgactcactataggcgtagcctgatgaggttaccccgaaacggccctaactgcatcagcgaaacgtggtgaaagccacgtagctgcgcc

SEQ ID NO: 267 - DNA, 30 bp, Artificial Sequence
  Signaling Polynucleotide
gttaccccgaaacggccctaactgcatcag

SEQ ID NO: 268 - DNA, 37 bp, Artificial Sequence
  Signaling Polynucleotide
cugaugcgttaccccgaaacggccctaactgcatcag

SEQ ID NO: 269 - DNA, 37 bp, Artificial Sequence
  Signaling Polynucleotide
gttaccccgaaacggccctaactgcatcagggguaac

SEQ ID NO: 270 - DNA, 89 bp, Artificial Sequence
  Aptamer
taatacgactcactataggcgtagcctgatgagccgcatcaccacccaaaccaccgttcgaaacgtggtgaaagccacgtagctgcgcc

SEQ ID NO: 271 - DNA, 25 bp, Artificial Sequence
  Signaling Polynucleotide
ccgcatcaccacccaaaccaccgtt

SEQ ID NO: 272 - DNA, 46 bp, Artificial Sequence
  Signaling Polynucleotide
gcctgatgagccgcatcaccacccaaaccaccgttcgaaacgaggc

SEQ ID NO: 273 - DNA, 50 bp, Artificial Sequence
  Signaling Polynucleotide
ttccaatgatgagccgcatcaccacccaaaccaccgttcgaaacgaggaa

SEQ ID NO: 274 - DNA, 52 bp, Artificial Sequence
  Signaling Polynucleotide
tatacaatgatgagccgcatcaccacccaaaccaccgttcgaaacgagtaag

SEQ ID NO: 275 - DNA, 47 bp, Artificial Sequence
  Signaling Polynucleotide
uucctgatgagccgcatcaccacccaaaccaccgttcgaaauaggaa

SEQ ID NO: 276 - DNA, 53 bp, Artificial Sequence
  Signaling Polynucleotide
tggacaatgatgagccgcatcaccacccaaaccaccgttcgaaacgagtcaca

SEQ ID NO: 277 - DNA, 53 bp, Artificial Sequence
  Signaling Polynucleotide
tccacaatgatgagccgcatcaccacccaaaccaccgttcgaaacgagtgaga

SEQ ID NO: 278 - DNA, 89 bp, Artificial Sequence
  Aptamer
taatacgactcactataggcgtagcctgatgagcctgctccatccgcgccagcctcaccgaaacgtggtgaaagccacgtagctgcgcc

SEQ ID NO: 279 - DNA, 25 bp, Artificial Sequence
  Signaling Polynucleotide
cctgctccatccgcgccagcctcac

SEQ ID NO: 280 - DNA, 41 bp, Artificial Sequence
  Signaling Polynucleotide
cctgatgagcctgctccatccgcgccagcctcaccgaaacg

SEQ ID NO: 281 - DNA, 50 bp, Artificial Sequence
  Signaling Polynucleotide
tagcctgatgagcctgctccatccgcgccagcctcaccgaaacgtggcta

SEQ ID NO: 282 - DNA, 45 bp, Artificial Sequence
  Signaling Polynucleotide
cttagcctgatgagcctgctccatccgcgccagcctcaccgtaag

SEQ ID NO: 283 - DNA, 43 bp, Artificial Sequence
  Signaling Polynucleotide
cttagggtagagcctgctccatccgcgccagcctcaccutaag

SEQ ID NO: 284 - DNA, 41 bp, Artificial Sequence
  Signaling Polynucleotide
ttagggtagagcctgctccatccgcgccagcctcaccutaa

SEQ ID NO: 285 - DNA, 42 bp, Artificial Sequence
  Signaling Polynucleotide
gttagggtagagcctgctccatccgcgccagcctcaccuaau

SEQ ID NO: 286 - DNA, 39 bp, Artificial Sequence
  Signaling Polynucleotide
ttaggtagagcctgctccatccgcgccagcctcaccuaa

SEQ ID NO: 287 - DNA, 56 bp, Artificial Sequence
  Signaling Polynucleotide
gcgtagcctgatgagcctgctccatccgcgccagcctcaccgaaacgtggtgaaag

SEQ ID NO: 288 - DNA, 50 bp, Artificial Sequence
  Signaling Polynucleotide
taatacgaccctgctccatccgcgccagcctcaccgaaacgtggtgaaag

SEQ ID NO: 289 - DNA, 50 bp, Artificial Sequence
  Signaling Polynucleotide
tcactatagcctgctccatccgcgccagcctcaccgaaacgtggtgaaag

SEQ ID NO: 290 - DNA, 56 bp, Artificial Sequence
  Signaling Polynucleotide
ccacgtagctgcgcccctgctccatccgcgccagcctcaccgaaacgtggtgaaag

SEQ ID NO: 291 - DNA, 56 bp, Artificial Sequence
  Signaling Polynucleotide
cuutcacctgatgagcctgctccatccgcgccagcctcaccgaaacgtggtgaaag

SEQ ID NO: 292 - DNA, 56 bp, Artificial Sequence
  Signaling Polynucleotide
uautcacctgatgagcctgctccatccgcgccagcctcaccgaaacgtggtgaaag

SEQ ID NO: 293 - DNA, 41 bp, Artificial Sequence
  Signaling Polynucleotide
utcgatgagcctgctccatccgcgccagcctcaccgaacag

SEQ ID NO: 294 - DNA, 43 bp, Artificial Sequence
  Signaling Polynucleotide
cautcgatgagcctgctccatccgcgccagcctcaccgaacug

SEQ ID NO: 295 - DNA, 89 bp, Artificial Sequence
  Aptamer
taatacgactcactataggcgtagcctgatgagccaatctcctgcccacgccgttccacgaaacgtggtgaaagccacgtagctgcgcc

SEQ ID NO: 296 - DNA, 25 bp, Artificial Sequence
  Signaling Polynucleotide
ccaatctcctgcccacgccgttcca

SEQ ID NO: 297 - DNA, 59 bp, Artificial Sequence
  Signaling Polynucleotide
taggcgtagcctgatgagccaatctcctgcccacgccgttccacgaaacgtggtgaaag

SEQ ID NO: 298 - DNA, 47 bp, Artificial Sequence
  Signaling Polynucleotide
taggcgtagcctgatgagccaatctcctgcccacgccgttccacgaa

SEQ ID NO: 299 - DNA, 53 bp, Artificial Sequence
  Signaling Polynucleotide
taggcgtagcctgatgagccaatctcctgcccacgccgttccaccuacgccua

SEQ ID NO: 300 - DNA, 47 bp, Artificial Sequence
  Signaling Polynucleotide
gcgtagcctgatgagccaatctcctgcccacgccgttccaccuacgc

SEQ ID NO: 301 - DNA, 47 bp, Artificial Sequence
  Signaling Polynucleotide
ucgtagcctgatgagccaatctcctgcccacgccgttccaccuacca

SEQ ID NO: 302 - DNA, 47 bp, Artificial Sequence
  Signaling Polynucleotide
ugctagcctgatgagccaatctcctgcccacgccgttccaccuagaa

SEQ ID NO: 303 - DNA, 47 bp, Artificial Sequence
  Signaling Polynucleotide
cgatagcctgatgagccaatctcctgcccacgccgttccaccuatag

SEQ ID NO: 304 - DNA, 55 bp, Artificial Sequence
  Signaling Polynucleotide
gucctagcctgatgagccaatctcctgcccacgccgttccacaucuugcuagaac

SEQ ID NO: 305 - DNA, 41 bp, Artificial Sequence
  Signaling Polynucleotide
gatgagccaatctcctgcccacgccgttccacaagcucauc

SEQ ID NO: 306 - DNA, 44 bp, Artificial Sequence
  Signaling Polynucleotide
cctgatgagccaatctcctgcccacgccgttccacaucgaaagg

SEQ ID NO: 307 - DNA, 89 bp, Artificial Sequence
  Aptamer
taatacgactcactataggcgtagcctgatgagccaatcaaggaccgccttcaccgctcgaaacgtggtgaaagccacgtagctgcgcc

SEQ ID NO: 308 - DNA, 25 bp, Artificial Sequence
  Signaling Polynucleotide
ccaatcaaggaccgccttcaccgct

SEQ ID NO: 309 - DNA, 41 bp, Artificial Sequence
  Signaling Polynucleotide
cctgatgagccaatcaaggaccgccttcaccgctcgaaacg

SEQ ID NO: 310 - DNA, 35 bp, Artificial Sequence
  Signaling Polynucleotide
gatgagccaatcaaggaccgccttcaccgctcgau

SEQ ID NO: 311 - DNA, 31 bp, Artificial Sequence
  Signaling Polynucleotide
tgagccaatcaaggaccgccttcaccgctcg

SEQ ID NO: 312 - DNA, 34 bp, Artificial Sequence
  Signaling Polynucleotide
gcacagccaatcaaggaccgccttcaccgctucu

SEQ ID NO: 313 - DNA, 35 bp, Artificial Sequence
  Signaling Polynucleotide
gcaacagccaatcaaggaccgccttcaccgctuuu

SEQ ID NO: 314 - DNA, 33 bp, Artificial Sequence
  Signaling Polynucleotide
gacagccaatcaaggaccgccttcaccgctuuu

SEQ ID NO: 315 - DNA, 89 bp, Artificial Sequence
  Aptamer
taatacgactcactataggcgtagcctgatgagactctcgcatcaccagccaactcaccgaaacgtggtgaaagccacgtagctgcgcc

SEQ ID NO: 316 - DNA, 25 bp, Artificial Sequence
  Signaling Polynucleotide
actctcgcatcaccagccaactcac

SEQ ID NO: 317 - DNA, 53 bp, Artificial Sequence
  Signaling Polynucleotide
tagcctgatgagactctcgcatcaccagccaactcaccgaaacgtggtgaaag

SEQ ID NO: 318 - DNA, 53 bp, Artificial Sequence
  Signaling Polynucleotide
tagcctgatgagactctcgcatcaccagccaactcaccgaaacgtggtggcua

SEQ ID NO: 319 - DNA, 53 bp, Artificial Sequence
  Signaling Polynucleotide
tagccagatgagactctcgcatcaccagccaactcaccgaaacgtggtggcua

SEQ ID NO: 320 - DNA, 53 bp, Artificial Sequence
  Signaling Polynucleotide
tagccagatgagactctcgcatcaccagccaactcaccgaacacauctggcua

SEQ ID NO: 321 - DNA, 53 bp, Artificial Sequence
  Signaling Polynucleotide
tcgccagatgagactctcgcatcaccagccaactcaccgaaacgtggtggcga

SEQ ID NO: 322 - DNA, 53 bp, Artificial Sequence
  Signaling Polynucleotide
tcgccagatgagactctcgcatcaccagccaactcaccgaaacgtggaggcga

SEQ ID NO: 323 - DNA, 53 bp, Artificial Sequence
  Signaling Polynucleotide
tugccagatgagactctcgcatcaccagccaactcaccgaaacgtggaggcga

SEQ ID NO: 324 - DNA, 55 bp, Artificial Sequence
  Signaling Polynucleotide
tugccagatgagactctcgcatcaccagccaactcaccgaaacgtggaguugcga

SEQ ID NO: 325 - DNA, 54 bp, Artificial Sequence
  Signaling Polynucleotide
tugccuugatgagactctcgcatcaccagccaactcaccgaaacgtggaggcga

SEQ ID NO: 326 - DNA, 55 bp, Artificial Sequence
  Signaling Polynucleotide
tugccuaugatgagactctcgcatcaccagccaactcaccgaaacgtggaggcga

SEQ ID NO: 327 - DNA, 55 bp, Artificial Sequence
  Signaling Polynucleotide
tugccugatgagactctcgcatcaccagccaactcaccgaaacgaatggaggcga

SEQ ID NO: 328 - DNA, 54 bp, Artificial Sequence
  Signaling Polynucleotide
tugccugatgagactctcgcatcaccagccaactcaccgaaacggtggaggcga

SEQ ID NO: 329 - DNA, 53 bp, Artificial Sequence
  Signaling Polynucleotide
tugccugatgagactctcgcatcaccagccaactcaccgaaacgtggaggcat

SEQ ID NO: 330 - DNA, 53 bp, Artificial Sequence
  Signaling Polynucleotide
tugccugatgagactctcgcatcaccagccaactcaccgaaacgtggaggcaa

SEQ ID NO: 331 - DNA, 89 bp, Artificial Sequence
  Aptamer
taatacgactcactataggcgtagcctgatgagcggtactcagattacagagtgacatcgaaacgtggtgaaagccacgtagctgcgcc

SEQ ID NO: 332 - DNA, 25 bp, Artificial Sequence
  Signaling Polynucleotide
cggtactcagattacagagtgacat

SEQ ID NO: 333 - DNA, 88 bp, Artificial Sequence
  Aptamer
taatacgactcactataggcgtagcctgatgagagacaccacggatccgaactggagcgaaacgtggtgaaagccacgtagctgcgcc

SEQ ID NO: 334 - DNA, 24 bp, Artificial Sequence
  Signaling Polynucleotide
agacaccacggatccgaactggag

SEQ ID NO: 335 - DNA, 89 bp, Artificial Sequence
  Aptamer
taatacgactcactataggcgtagcctgatgagcctcgcaagattgcatacgttagaacgaaacgtggtgaaagccacgtagctgcgcc

SEQ ID NO: 336 - DNA, 25 bp, Artificial Sequence
  Signaling Polynucleotide
cctcgcaagattgcatacgttagaa

SEQ ID NO: 337 - DNA, 89 bp, Artificial Sequence
  Aptamer
taatacgactcactataggcgtagcctgatgagcacgtaggaaacgacctctacggagcgaaacgtggtgaaagccacgtagctgcgcc

SEQ ID NO: 338 - DNA, 25 bp, Artificial Sequence
  Signaling Polynucleotide
cacgtaggaaacgacctctacggag

SEQ ID NO: 339 - DNA, 90 bp, Artificial Sequence
  Aptamer
taatacgactcactataggcgtagcctgatgagcccgaaaccaccaccgttgtccaatacgaaacgtggtgaaagccacgtagctgcgcc

SEQ ID NO: 340 - DNA, 26 bp, Artificial Sequence
  Signaling Polynucleotide
cccgaaaccaccaccgttgtccaata

SEQ ID NO: 341 - DNA, 40 bp, Artificial Sequence
  Signaling Polynucleotide
ccagtctcccgtttaccgcgcctacacatgtctgaatgcc

SEQ ID NO: 342 - DNA, 37 bp, Artificial Sequence
  Signaling Polynucleotide
tagtctcccgtttaccgcgcctacacatgtctgaatg

SEQ ID NO: 343 - DNA, 35 bp, Artificial Sequence
  Signaling Polynucleotide
tagtctcccgtttaccgcgcctacacatgtctgaa

SEQ ID NO: 344 - DNA, 40 bp, Artificial Sequence
  Signaling Polynucleotide
ctaggcgaaatatagctacaactgtctgaaggcacccaat

SEQ ID NO: 345 - DNA, 42 bp, Artificial Sequence
  Signaling Polynucleotide
atctacgaattcatcagggctaaagagtgcagagttacttag

SEQ ID NO: 346 - DNA, 36 bp, Artificial Sequence
  Signaling Polynucleotide
tacgaattcatcagggctaaagagtgcagagttact

SEQ ID NO: 347 - DNA, 48 bp, Artificial Sequence
  Signaling Polynucleotide
aatatctacgaattcatcagggctaaagagtgcagagttacttagctc

SEQ ID NO: 348 - DNA, 42 bp, Artificial Sequence; identical to SEQ ID NO: 345
  Signaling Polynucleotide

SEQ ID NO: 349 - DNA, 40 bp, Artificial Sequence
  Signaling Polynucleotide
gcagctaagcaggcggctcacaaaaccattcgcatgcggc

SEQ ID NO: 350 - DNA, 34 bp, Artificial Sequence
  Signaling Polynucleotide
gctaagcaggcggctcacaaaaccattcgcatgc

SEQ ID NO: 351 - DNA, 36 bp, Artificial Sequence
  Signaling Polynucleotide
gctaagcaggcggctcacaaaaccattcgcatgcaa

SEQ ID NO: 352 - RNA, 30 nt, Artificial Sequence
  Signaling Polynucleotide
gguugugaagauugggagcgucguggcuac

SEQ ID NO: 353 - DNA, 80 bp, Artificial Sequence
  Signaling Polynucleotide
tcgcacattccgcttctaccgggggggtcgagctgagtggatgcgaatctgtgggtgggccgtaagtccgtgtgtgcgaa

(19) United States
(12) Patent Application Publication
(10) Pub. No.: US 2019/0119669 A1
(43) Pub. Date: Apr. 25, 2019

(54) ALLERGEN DETECTION USING MAGNETICS

(71) Applicant: DOTS Technology Corp., Natick, MA (US)

(72) Inventors: Adi Gilboa-Geffen, Wayland, MA (US); Renuka Babu Brown, Weston, MA (US)

(21) Appl. No.: 16/093,200

(22) PCT Filed: Apr. 11, 2017
(86) PCT No.: PCT/US17/26892

§ 371 Date: Oct. 12, 2018

Related U.S. Application Data
(60) Provisional application No. 62/321,642, filed on Apr. 12, 2016.

(51) Int. Cl. C12N 15/10; C12Q 1/6811; G01N 33/543

(52) U.S. Cl. CPC C12N 15/10; G01N 33/54326; C12Q 1/6811

(57) ABSTRACT

The present invention relates to assays and methods for target analyte (e.g., allergen) detection in a sample using aptamer-magnetic particle complexes.

### CROSS REFERENCE TO RELATED APPLICATIONS

This application claims priority to U.S. Provisional Application Ser. No. 62/321,642 filed on Apr. 12, 2016, which is entitled “Allergen Detection Using Magnetics”; the contents of which are incorporated herein by reference in their entirety.

### REFERENCE TO THE SEQUENCE LISTING

The present application is being filed along with a Sequence Listing in electronic format. The Sequence Listing is provided as a file entitled 20661006PCTSEQLST.txt, created on Apr. 11, 2017, which is 76,975 bytes in size. The information in the electronic format of the sequence listing is incorporated herein by reference in its entirety.

### FIELD OF THE INVENTION

The present invention relates to aptamer-magnetic particle conjugates for use in allergen detection.

### BACKGROUND OF THE INVENTION

Allergy is a serious medical condition affecting millions of people worldwide, with about 15 million people in the United States, including many children. During an allergic reaction, the immune system mistakenly targets an allergen as a threat and attacks it. The allergic reaction may affect the skin, the digestive system, the gastrointestinal tract, the respiratory system, the circulatory system and the cardiovascular system; in some allergic reactions, multiple organ systems are affected. Allergic reactions range from mild to severe or life-threatening. Severe symptoms may include difficulty in breathing, low blood pressure, chest pain, loss of consciousness, and anaphylaxis. People having allergies currently manage their allergies by avoiding any food that might contain that specific allergen. These restrictions have a major impact on the patients' quality of life and there remains no method for assessing the true allergen content of food. In the United States, food allergy symptoms send someone to the emergency room every three minutes. Given the fact that antibodies to allergens are not easily to obtained, there is an unmet demand of new platform technology to improve in vitro allergen detection in both clinical and non-clinical settings.

Antibody-based immunoassays are commonly used for food allergen detection. Several technologies for detecting the antibody-antigen (i.e. allergen) complexes have been developed including signaling molecules attached to antibodies, signal collection and calculation, and systems and devices to implement the detection assays.

Recently, nucleic acid aptamers and SELEX technology have gained great attention. Aptamers are excellent alternatives or supplements to antibodies including monoclonal antibodies. As compared to antibodies, aptamers are cost-effective. Their small sizes and nucleic acid characteristics also improve their suitability for industrialization. Moreover, aptamers can be developed against a seemingly unlimited range of targets such as small inorganic ions, drugs, organic peptides, proteins and even complex cells. Aptamers are thermally stable, so they can be stored and transported easily. These properties make aptamers good agents for analyte detection in a sample, protein/nucleic acid purification and other aspects of biological researches.

For example, for food allergen detection, in addition to antibodies, nucleic acid based aptamers recently have been approved to be reliable and ultrasensitive agents for detecting food allergen because of the high sensitivity and specificity of aptamers to target allergens (Nadal, et al., DNA aptamers against the Lup ani food allergen, Plos One, 2012, 7(4): e35253; Amaya-Gonzalez et al., Aptamer-based analysis: A promising alternative for food safety control, Sensor, 2013, Vol 13, pages 16292-16311; Svobodova et al., Ultrasensitive aptamer based detection of β-conglutin food allergen, Food Chemistry, 2014, Vol 165, Pages 419-423; and Trashin et al., Label-Free Impedance Aptasensor for Major Peanut Allergen Ara h 1, Electroanalysis, 2015, Vol 27(1), Pages 32-37).

Aptamer based biosensors for rapid, sensitive and highly selective detection of a target allergen have been developed and disclosed in the prior art for a variety of application. A rapid and sensitive detection of aptamer-target complexes in an aptamer based assay is critical to these aptamer-based biosensors. Similar to antibodies, aptamers can be labeled with fluorophores, enzymes, redox compounds and other signaling detectors, covalently or noncovalently. Recent studies have also reported that magnetic particles conjugated to nucleic acid aptamers can be used to detect analytes in biological samples, to purify proteins and to study protein-to-protein interaction. Magnetic detection is simple and easy to operation. Several patents and patent applications have disclosed how magnetic particles, when used as detection agents for analyte detection, can be easily detected and transmitted into the signals indicative of the presence, absence and/or quantity of target analytes in samples, for example, see U.S. Pat. Nos. 9,207,245; 9,207,244; 9,086,417; 9,052,275; and U.S. Pat. No. 9,034,168 to Ayub et al; U.S. Pat. Nos. 9,244,068 and 8,895,320 to Florescu et al.; and U.S. Pat. No. 8,614,572 to Florescu; U.S. Patent Application Publication NOs.: US2014/0336083 to Ayub et al; US2015/0129049 to Ayub et al.; and US2013/0230913 to Florescu; and PCT Patent Application NOs.: WO.2014/189624 to Florescu et al; the contents of each of which are herein incorporated by reference in their entirety.

In the present application, applicant identifies aptamers that bind common allergens with high specificity and affinity, and develops aptamer-magnetic particle conjugates used for allergen detection. Such aptamer-magnetic particle conjugates may be used as reagents for allergen detection in a variety of analyte detection assays, kits, devices and systems.

### SUMMARY OF THE INVENTION

The present invention provides methods for detecting target allergens in a sample dependent on aptamers with nucleic acid sequences that bind allergens with high specificity and affinity. Aptamer derived signaling polynucleotides of the present invention may be conjugated to magnetic particles, such aptamer-magnetic particle complexes can be used reagents in various assays, biosensors and systems for detection of target analyte (e.g., allergen) in a sample. In some aspects, the signaling polynucleotides comprise nucleic acid sequences of SEQ ID NOs.:1-353, which bind specifically to eight common food allergens.

In some embodiments, methods of the present invention for detecting the absence, presence and/or quantity of an allergen in a test sample comprise: (a). obtaining a test sample which is suspected to contain the target allergen; (b). placing the test sample into a sample analysis cartridge, wherein the cartridge comprises an input tunnel configured for receiving the test sample, a plurality of reservoirs which separately store sample preparation reagents and a substrate, and an analysis channel; (c). mixing the test sample with the sample preparation reagents stored in the reservoirs sequentially from the first reservoir, the second reservoir and the third reservoir, and so on, wherein the target allergen is hybridized with the preparation reagents; (d). initiating a testing protocol in a specialized computer which is configured to detect the sample analysis cartridge; (e). releasing the contents of the plurality of reservoirs into the analysis channel of the sample analysis cartridge wherein one or more sensors are disposed on the analysis channel to detect the hybridized target allergen; and (f). processing and analyzing the detection signals to identify the absence, presence, and/or quantity of the target allergen in the test sample. The detection devices and systems implementing the present assays may include those described by Ayub et al. in the PCT patent application publication No.: WO2014/164933; U.S. Pat. Nos. 9,207,245; 9,207,244; 9,086,417; 9,034,168; and 9,052,275; and U.S. patent application publication No.: US 2014/0336083.

In other embodiments, methods of the present invention for detecting the absence, presence and/or quantity of a target allergen in a test sample comprise: (a) obtaining a test sample which is suspected to contain the target allergen; (b) filtering the test sample using a filter configured to filter the test sample resulting in a filtrate comprising the target allergen; (c) delivering the filtrate of step (b) through a capillary to a surface of an integrated circuit comprising one or more sensor areas on the surface of said integrated circuit, wherein dried magnetic particles whose surfaces are functionalized to react with one or more target allergens in the filtrate are pre-stored in the capillary channel or the sensor areas on the surface of the integrated circuit, and wherein the filtrate flows in the capillary channel and target allergens in the filtrate bind the functionalized magnetic particles to form target magnetic particle complexes which can bind specifically onto the sensor areas on the surface of the integrated circuit; (d) detecting magnetic particles specifically bound to said one or more sensor areas on the surface of the integrated circuit using a plurality of sensors; and (e) transmitting signals detected in step (d) into indicative of the absence, presence and/or quantity of the target allergen in the test sample.

### BRIEF DESCRIPTION OF THE DRAWINGS

FIG. 1A displays the conjugation of biotinalyzed SPN to streptavidin coated magnetic particles to form SPN-magnetic particle complexes.

FIG. 1B depicts target allergen capture using SPN-magnetic particle complexes as detection agents.

FIG. 2A, FIG. 2B, FIG. 2C and FIG. 2D represent PRIOR ART figures from PCT patent publication NO.: WO2014/164933, FIGS. 1A-1D, respectively.

FIG. 3 represents a PRIOR ART figure from U.S. Pat. No. 9,244,068, FIG. 8.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### Composition of the Present Invention

### Aptamers

Molecules that recognize others with extreme specificity and high-affinity are important for a wide range of applications such as detection of analytes in samples. Typically, antibodies fulfill this role in immunoassays. Recent advancement of research has led to the discovery of a class of oligonucleotides referred to as aptamers that can recognize molecules with high-affinity and specificity. Consequently, aptamers have the potential to fulfill the role that antibodies play in research applications including analyte detection. One of the more recent reviews of aptamer-based analysis in context of food safety control indicated that the selection of aptamers for this group of ingredients is emerging (Amaya-González et al., Sensors 2013, 13: 16292-16311, the contents of which are incorporated herein by reference in its entirety).

Aptamers (sometimes also called chemical antibodies) are single-stranded oligonucleotides (RNA or single stranded DNA) that form stable but unique three-dimensional confirmations capable of binding with high affinity and specificity to a variety of molecular targets. Aptamers bind to protein targets in much the same manner as antibodies and modulate protein function. Thus, aptamers are also referred to as “chemical antibodies”. Generally, aptamers can be selected from random-sequence, single-stranded nucleic acid libraries by an in vitro selection and amplification procedure known as SELEX (systematic evolution of ligands by exponential enrichment). The selected aptamers are small single-stranded nucleic acids that fold into a well-defined three-dimensional structure. They show a high affinity and specificity for their target molecules and inhibit their biological functions.

Aptamers have advantages over antibodies in that they are poorly immunogenic, stable, and often bind to a target molecule more strongly than do antibodies. It is possible to produce an aptamer with a high affinity for a small molecule, such as a peptide or other molecular compound, against which antibodies are difficult to obtain. Producing an aptamer is more cost-advantageous than an antibody because it can be synthesized easily and in large quantities by in vitro transcription, PCR, or chemical synthesis (Annu. Rev. Med. 2005, 56, 555-583; Nat. Rev. Drug Discov. 2006, 5, 123-132). Thus, aptamers are useful and cost-effective tools for biochemical analyses. Also, they can be developed quickly against a seemingly unlimited range of targets. To date, specific aptamers against diverse targets have been successfully developed, including small inorganic irons, organic peptides, drugs, proteins, lipids and even complex cells. Furthermore, aptamers have important properties that simplify its industrialization. For example, aptamers are thermally stable, so they can be stored and transported easily. Aptamers can be produced or modified in large scale, with minimal batch-to-batch variation, given the well-established chemical synthesis and modification technologies.

As compared to other detection agents, aptamers can be conjugated to many particles due to their high suitability and flexibility. Moreover, aptamers are more amenable to chemical modifications, making them capable of utilization with most developed sensors. The present inventors have recognized that production of signaling polynucleotides (SPNs) (described in detail herein below) using an aptamer as the core/binding sequence allows convenient linkage to various detectors, such as fluorophores, enzymes, metal nanoparticles, redox compounds and magnetic particles.

The relatively low production cost of signaling polynucleotides based on aptamer core/binding sequences is also advantageous with respect to the objective of development of simple, yet effective detection assays for biomolecule sensors. A method of detection of gluten is described in PCT Publication PCT/ES2013/000133, 28 Jun. 2013, to Amaya-Gonzalez, et al; the contents of which are incorporated herein by reference in its entirety. By way of non-limiting example, a process for in vitro selection of a single stranded DNA aptamer specific for the anaphylactic toxic allergen, β-conglutin, Lup an 1 has been reported (Nadal, et al., PLoS ONE, 2012, 7(4): e35253; and Nadal, et al., Anal. Bioanal. Chem. 2013, 405: 9343-9349; the contents of each of which are incorporated herein by reference in their entirety).

A sensitive method exploiting fluorescence resonance energy transfer (FRET) was recently reported for rapid and sensitive detection of Lup an 1, using a high affinity dimeric form of the truncated 11-mer anti-β-conglutin aptamer, with each monomeric aptamer being flanked by donor/acceptor moieties. The dimeric form in the absence of target yields fluorescence emission due to the FRET from the excited fluorophore to the proximal second fluorophore. However, upon addition of β-conglutin, the specific interaction induces a change in the bi-aptameric structure resulting in an increase in fluorescence emission. The method is highly specific and sensitive, with a detection limit of 150 pM, providing an effective tool for the direct detection of the toxic β-conglutin subunit in foodstuffs in just 1 min. at room temperature (Mairal, et al., Biosensors and Bioelectronics, 2014, 54: 207-210; the contents of which are incorporated by reference herein in its entirety).

The present inventors have recognized that allergen detection in various matrices of food products can be conveniently performed using aptamer-based detector sequences such as signaling polynucleotides, which are particularly well suited for use in a simple and portable sensor that can be used repetitively with high sensitivity and reproducibility at ambient temperature to ensure food safety.

### Selection of Aptamers for a Target

Aptamers can be artificially generated by a method called systematic evolution of ligands by exponential enrichment (SELEX) (Science, 1990, 249, 505-510). More recently, a new improved separation technology for aptamer selection was introduced, capillary electrophoresis (CE)-SELEX.

Aptamers that bind to virtually any particular target can be selected by using an iterative process called SELEX™ (Systemic Evolution of Ligands by Exponential Enrichment). The process is described in, for example U.S. Pat. Nos. 5,270,163 and 5,475,096. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric.

The SELEX™ process relies, as a starting point, upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected purpose such as, CpG motifs, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically, the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in the test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs (see for example U.S. Pat. Nos. 5,958,691 and 5,660,985). Random oligonucleotides can be synthesized from phosphodiester-linked nucleotides using solid phase oligonucleotide synthesis techniques well known in the art. Random oligonucleotides can also be synthesized using solution phase methods such as triester synthesis methods. Typical syntheses carried out on automated DNA synthesis equipment yield 10¹⁴-10¹⁶ individual molecules, a number sufficient for most SELEX™ experiments.

The starting library of oligonucleotides may be generated by automated chemical synthesis on a DNA synthesizer. Partially random sequences can be created by adding the four nucleotides in different molar ratios at each addition step.

The library of oligonucleotides for aptamer selection may be either RNA or DNA. A RNA library of oligonucleotides is typically generated by transcribing a DNA library o foligonucleotides in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve the desired criterion of binding affinity and selectivity as defined in the present application.

More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. Cycles of selection and amplification are repeated until a desired goal is achieved. Generally this is until no significant improvement in binding strength is achieved on repetition of the cycle. Typically, nucleic acid aptamer molecules are selected in a 5 to 20 cycle procedure.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. The core SELEX™ method has been modified to achieve a number of specific objectives, such as selection of aptamers with particular secondary structures. Examples of SELEX processes can be found in U.S. Pat. Nos. 5,270,163 and 5,475,096. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-crosslinking to and/or photo-inactivating a target molecule U.S. Pat. Nos. 5,567,588 and 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target, the contents of each of which are incorporated herein by reference in their entirety.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

The binding affinity describes the measure of the strength of the binding or affinity of molecules to each other. Binding affinity of the aptamer herein with respect to targets and other molecules is defined in terms of K_{d}. The dissociation constant can be determined by methods known in the art. It has been observed, however, that for some small oligonucleotides, direct determination of K_{d} is difficult, and can lead to misleadingly high results. Under these circumstances, a competitive binding assay for the target molecule or other candidate substance can be conducted with respect to substances known to bind the target or candidate. The value of the concentration at which 50% inhibition occurs (K,) is, under ideal conditions, equivalent to K_{d}.

In accordance with the present invention, a SELEX approach was used to select core binding aptamers that bind 8 major food allergens (i.e. cashew, egg, milk, peanuts, gluten, fish, crustacean and soy). Several aptamers with sequences that can specifically recognize a target allergen were selected and the nucleic acid sequences of selected aptamers were further modified to generate signaling polynucleotides. The aptamers with high selectivity, specificity and stability are selected and further labeled as detection agents. The sequences of the selected aptamers for the 8 major allergens are listed in Table 1. For example, 1501 RiboSPN (SEQ ID NO.: 1) is the full sequence of one of the aptamers that bind cashew. The full sequence includes the primers used for the screen and the core binding sequence of the aptamer (SEQ ID NO.: 2). the full sequence will be further modified to generate signaling polynucleotides specific to cashew, as discussed herein below.

### Aptamer Modifications

In accordance with the present invention, oligonucleotides and aptamers may be further modified to improve their stability. The present invention also includes analogs as described herein and/or additional modifications designed to improve one or more characteristics of aptamers such as protection from nuclease digestion. Oligonucleotide modifications contemplated in the present invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole.

Modifications to generate oligonucleotides which are resistant to nucleases can also include one or more substitute internucleotide linkages, altered sugars, altered bases, or combinations thereof. Such modifications include 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, and unusual base-pairing combinations such as the isobases isocytidine and isoguanosine; 3′ and 5′ modifications such as capping; conjugation to a high molecular weight, non-immunogenic compound; conjugation to a lipophilic compound; and phosphate backbone modification.

Nucleic acid aptamers may be ribonucleic acid, deoxyribonucleic acid, or mixed ribonucleic acid and deoxyribonucleic acid. Aptamers may be single stranded ribonucleic acid, deoxyribonucleic acid or mixed ribonucleic acid and deoxyribonucleic acid.

Nucleic acid aptamers comprise a series of linked nucleosides or nucleotides. The term “nucleic acid,” in its broadest sense, includes any compound and/or substance that comprise a polymer of nucleotides. These polymers are often referred to as polynucleotides. Exemplary nucleic acid molecules or polynucleotides of the invention include, but are not limited to, either D- or L-nucleic acids, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure, the ribofuranosyl ring or in the ribose-phosphate backbone.

In some embodiments, the aptamer comprises at least one chemical modification. In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In a preferred embodiment, the high molecular weight, non-immunogenic compound is polyalkylene glycol, and more preferably is polyethylene glycol (PEG). The process of covalent conjugation of PEG to another molecule, normally a drug or therapeutic protein is known as PEGylation. PEGylation is routinely achieved by incubation of a reactive derivative of PEG with the target molecule. The covalent attachment of PEG to a drug or therapeutic protein can mask the agent from the host's immune system, thereby providing reduced immunogenicity and antigenicity, and increase the hydrodynamic size (size in solution) of the agent which prolongs its circulatory time by reducing renal clearance. PEGylation can also provide water solubility to hydrophobic drugs and proteins.

In another preferred embodiment, the 3′ cap is an inverted deoxythymidine cap.

In some embodiments, nucleic acid aptamers are provided in which the P(O)O group is replaced by P(O)S (“thioate”), P(S)S (“dithioate”), P(O)NR2 (“amidate”), P(O)R, P(O)OR′, CO or CH2 (“formacetal”) or 3′-amine (—NH—CH2-CH2-), wherein each R or R′ is independently H or substituted or unsubstituted alkyl. Linkage groups can be attached to adjacent nucleotide through a —O—, —N—, or —S— linkage. Not all linkages in the nucleic acid aptamers are required to be identical.

As non-limiting examples, a nucleic acid aptamer can include D-ribose or L-ribose nucleic acid residues and can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, an inverted deoxynucleoside or inverted ribonucleoside, a 2′-deoxy-2′-fluoro-modified nucleoside, a 2′-amino-modified nucleoside, a 2′-alkyl-modified nucleoside, a morpholino nucleoside, a phosphoramidate or a non-natural base comprising nucleoside, or any combination thereof. Alternatively, a nucleic acid aptamer can comprise at least two modified ribonucleosides, at least 3, at least 4, at least 5, at least 6, at least 7, at least 8, at least 9, at least 10, at least 15, at least 20 or more modified ribonucleosides, up to the entire length of the molecule. The modifications need not be the same for each of such a plurality of modified deoxy- or ribonucleosides in a nucleic acid molecule.

Aptamer may comprise modified nucleobase (often referred to in the art simply as “base”) for increasing the affinity and specificity for their target protein. As used herein, “unmodified” or “natural” nucleobases include the purine bases adenine (A) and guanine (G), and the pyrimidine bases thymine (T), cytosine (C) and uracil (U). For example, the modified base may be a pyrimidine modified by a hydrophobic group, such as benzyl group, a naphthyl group, or a pyrrolebenzyl group, at its 5-position. Modified nucleoside may be exemplified as 5-(N-benzylcarboxyamide)-2′-deoxyuridine (called BzdU), 5-(N-naphthylcarboxyamide)-2′-deoxyuridine (called NapdU), 5-(N-4-pyrrolebenzyl carboxyamide)-2′-deoxyuridine (called 4-PBdU), 5-(N-benzylcarboxyamide)-2′-deoxycytidine (called BzdC), 5-(N-naphthylcarboxyamide)-2′-deoxycytidine (called NapdC), 5-(N-4-pyrrolebenzylcarboxyamide)-2′-deoxycytidine (called 4-PBdC), 5-(N-benzylcarboxyamide)-2′-uridine (called BzU), 5-(N-naphthylcarboxyamide)-2′-uridine (called NapU), 5-(N-4-pyrrolebenzylcarboxyamide)-2′-uridine (called 4-PBU), 5-(N-benzylcarboxyamide)-2′-cytidine (called BzC), 5-(N-naphthylcarboxyamide)-2′-cytidine (called NapC), 5-(N-4-pyrrolebenzyl carboxyamide)-2′-cytidine (called 4-PBC), and the like, but not be limited thereto. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, those disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993.

In accordance with the present invention, a suitable nucleotide length for an aptamer ranges from about 15 to about 100 nucleotides (nt), and in various other preferred embodiments, 15-30 nt, 20-25 nt, 30-100 nt, 30-60 nt, 25-70 nt, 25-60 nt, 40-60 nt, 25-40 nt, 30-40 nt, any of 18, 19, 20, 21, 22, 23, 24, 25, 26, 27, 28, 29, 30, 31, 32, 33, 34, 35, 36, 37, 38, 39 or 40 nt or 40-70 nt in length. In some embodiments, an aptamer may be 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nt in length. In other embodiments, an aptamer may 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nt in length. However, the sequence can be designed with sufficient flexibility such that it can accommodate interactions of aptamers with two targets at the distances described herein.

In some embodiments, the nucleic acid aptamer comprises one or more regions of double-stranded character. Such double stranded regions may arise from internal self-complementarity or complementarity with a second or further aptamers or oligonucleotide molecule. In some embodiments, the double stranded region may be from 4-12, 4-10, 4-8 base pairs in length. In some embodiments, the double stranded region may be 5, 6, 7, 8, 9, 10, 11 or 12 base pairs. In some embodiments, the double stranded region may form a stem region. Such extended stem regions having double stranded character can serve to stabilize the nucleic acid aptamer. As used herein, the term “double stranded character” means that over any length of two nucleic acid molecules, their sequences form base pairings (standard or nonstandard) of more than 50 percent of the length.

Aptamers may be further modified to provide protection from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamers. For RNA aptamers, modified nucleotides such as substitutions of the 2′-OH groups of the ribose backbone, e.g., with 2′-deoxy-NTP or 2′-fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

In some embodiments, such modified nucleic acid aptamers may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modifications. For example, all purine nucleotides may have one type of modification (or are unmodified), while all pyrimidine nucleotides have another, different type of modification (or are unmodified). In this way, oligonucleotides, or libraries of oligonucleotides are generated using any combination of modifications as disclosed herein.

Aptamers may be either monovalent or multivalent. Aptamers may be monomeric, dimeric, trimeric, tetrameric or higher multimeric. Individual aptamer monomers may be linked to form multimeric aptamer fusion molecules. As a non-limiting example, a linking oligonucleotide (i.e., linker) may be designed to contain sequences complementary to both 5′-arm and 3′-arm regions of random aptamers to form dimeric aptamers. For trimeric or tetrameric aptamers, a small trimeric or tetrameric (i.e., a Holiday junction-like) DNA nanostructure will be engineered to include sequences complementary to the 3′-arm region of the random aptamers, therefore creating multimeric aptamer fusion through hybridization. In addition, 3 to 5 or 5 to 10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

As used herein, the term “multimeric aptamer” or “multivalent aptamer” refers to an aptamer that comprises multiple monomeric units, wherein each of the monomeric units can be an aptamer on its own. Multivalent aptamers have multivalent binding characteristics. A multimeric aptamer can be a homomultimer or a heteromultimer. The term “homomultimer” refers to a multimeric aptamer that comprises multiple binding units of the same kind, i.e., each unit binds to the same binding site of the same target molecule. The term “heteromultimer” refers to a multimeric aptamer that comprises multiple binding units of different kinds, i.e., each binding unit binds to a different binding site of the same target molecule, or each binding unit binds to a binding site on different target molecule. Thus, a heteromultimer can refer to a multimeric aptamer that binds to one target molecule at different binding sties or a multimeric aptamer that binds to different target molecules. A heteromultimer that binds to different target molecules can also be referred to as a multi-specific multimer.

According to certain embodiments of the present invention, variants and derivatives of aptamers are provided. The term “derivative” is used synonymously with the term “variant” and refers to a molecule that has been modified or changed in any way relative to a reference or starting aptamer. The nucleic acid sequence of aptamer variants may possess substitutions, deletions, and/or insertions at certain positions within the nucleotide sequence, as compared to a reference or starting sequence. Ordinarily, variants will possess at least about 50% identity (homology) to a reference sequence, and preferably, they will be at least about 80%, more preferably at least about 90% identical (homologous) to a reference sequence.

In some embodiments, variant mimics of aptamers of the present invention are provided. As used herein, the term “variant mimic” is one which contains one or more nucleic acids which would mimic an activated sequence. The nucleic acid sequences of variant mimics may comprise naturally occurring nucleic acids, or alternatively, non-naturally occurring nucleic acids.

### Aptamers as Signaling Polynucleotides (SPNs)

Aptamers selected through the process mentioned above herein may be used as signaling polynucleotides (SPNs) for detection of target allergens. In accordance with the present invention, a signaling polynucleotide may be developed from the selected aptamers which specifically bind a target allergen molecule. The polynucleotide sequences are detectable when bound at high affinity and specificity to molecular targets.

In some embodiments, signaling polynucleotides (SPNs) of the present invention comprise the core binding sequences which determine the specificity and affinity of SPNs to a target allergen molecule. The full sequence of a selected aptamer can be shortened by deleting the primers used for aptamer selection without impacting the binding sequence to a target allergen. Additional nucleotides may also be added at the 5′ terminus and/or the 3′ terminus, without impacting the binding (core) sequence of each aptamer. 3D structures of such SPNs are predicted using standard structure prediction software. The resulting polynucleotide may form a stable 3D structure. In other aspects, nucleotides added at the termini may increase the stability of the polynucleotide and facilitate magnetic particle conjugation. The length and sequence of additional nucleotides may vary in the context of the core binding sequence of a signaling polynucleotide. SPNs generated from aptamers against common allergens are listed in Table 1. For example, 1501-SPN_A (SEQ ID NO.: 3) and 1501 SPN_B (SEQ ID NO.: 4) are two polynucleotides derived from the aptamer 1501 RiboSPN (SEQ ID NO.: 1).

TABLE 1
Aptamers and SPNs that bind common allergens

| | | SEQ | |
|---|---|---|---|
| | | ID | |
| Allergen | SPN ID | NO. | Sequence (5′-3′) |
| Cashew | 1501RiboSPN_ | 1 | TAATACGACTCACTATAGGCGTAGCCTGATGAGGCACA |
| | full sequence | | CCACGTCAAAAATCATTGTCACCACGAAGCCGAAACG |
| | | | TGGTGAAAGCCACGT AGCTGCGCC |
| | 1501 SPN_core | 2 | GCACACCACGTCAAAAATCATTGTCACCACGAAGC |
| | sequence | | |
| | 1501 SPN_A | 3 | GCA |
| | | | GCACACCACGTCAAAAATCATTGTCACCACGAAGC |
| | | | TGC |
| | 1501 SPN_B | 4 | ATGCCGCACACCACGUCAAAAAUCAUUGUCACCACGA |
| | | | AGCGGCAT |
| | 1494 | 5 | TAATACGACTCACTATAGGCGTAGCCTGATGAGTGCGC |
| | RiboSPN_full | | AACATAAGTCTCTTGAAAGACCACGTTCAACGAAACG |
| | sequence | | TGGTGAAAGCCACGTAGCTGCGCC |
| | 1494 SPN_core | 6 | TGCGCAACATAAGTCTCTTGAAAGACCACGTTCAA |
| | sequence | | |
| | 1494 SPN_A | 7 | CCTGATGAGTGCGCAACATAAGTCTCTTGAAAGACCAC |
| | | | GTTCAACGAAA |
| | 1494 SPN_B | 8 | UUCCTGATGAGTGCGCAACATAAGTCTCTTGAAAGACC |
| | | | ACGTTCAA CGAAA |
| | 1494 SPN_C | 9 | UAACTGATGAGTGCGCAACATAAGTCTCTTGAAAGACC |
| | | | ACGTTCAACGUA |
| | 1494 SPN_D | 10 | UACGTGATGAG |
| | | | TGCGCAACATAAGTCTCTTGAAAGACCACGTTCAA |
| | | | CGUA |
| | 1494 SPN_E | 11 | ACATGATGAG |
| | | | TGCGCAACATAAGTCTCTTGAAAGACCACGTTCAA |
| | | | AUGU |
| | 1065 | 12 | TAATACGACTCACTATAGGCGTAGCCTGATGAGCACCC |
| | RiboSPN_full | | ACCATACCAGAAATGTTGACACCACGTGGACGAAACG |
| | sequence | | TGGTGAAAGCCACGTAGCTGCGCC |
| | 1065 SPN_core | 13 | CACCCACCATACCAGAAATGTTGACACCACGTGGA |
| | sequence | | |
| | 1065 SPN_A | 14 | CCTGATGAGCACCCACCATACCAGAAATGTTGACACCA |
| | | | CGTGGACGAAACGTGG |
| | 1065 SPN_B | 15 | UGUCACCCACCATACCAGAAATGTTGACACCACGTGG |
| | | | A CGACA |
| | 1065 SPN_C | 16 | UAUCACCCACCATACCAGAAATGTTGACACCACGTGG |
| | | | A GAUA |
| | 1065 SPN_D | 17 | UCGCACCCACCATACCAGAAATGTTGACACCACGTGG |
| | | | A AAACGA |
| | 1065 SPN_E | 18 | UCACACCCACCATACCAGAAATGTTGACACCACGTGG |
| | | | A AAAUGA |
| | 1065 SPN_F | 19 | UCGUCCCACCCACCATACCAGAAATGTTGACACCACGT |
| | | | GGAAAAACGA |
| | 1065 SPN_G | 20 | CACCCACCATACCAGAAATGTTGACACCACGTGGA |
| | | | CGAAACGTGGTGAAAG |
| | 1065 SPN_H | 21 | CUU |
| | | | CACCCACCATACCAGAAATGTTGACACCACGTGGA |
| | | | CGAAACGTGGTGAAAGAAG |
| | 1065 SPN_I | 22 | UU CACCCACCATACCAGAAATGTTGACACCACGTGGA |
| | | | CGAAACGTGGTGAAAGAA |
| | 1065 SPN_J | 23 | UA CACCCACCATACCAGAAATGTTGACACCACGTGGA |
| | | | CGAAACGTGGTGUGUA |
| | 1065 SPN_K | 24 | UA CACCCACCATACCAGAAATGTTGACACCACGTGGA |
| | | | CGAAACGTGGTGAUGUA |
| | 1065 SPN_L | 25 | UA CACCCACCATACCAGAAATGTTGACACCACGTGGA |
| | | | CGAAACGTGCAGUA |
| | 1904 | 26 | TAATACGACTCACTATAGGCGTAGCCTGATGAGTGCAC |
| | RiboSPN_full | | AATGTAATTATCAAAATACACCACGTTGGCCGAAACGT |
| | sequence | | GGTGAAAGCCACGTAGCTGCGCC |
| | 1904 SPN_core | 27 | TGCACAATGTAATTATCAAAATACACCACGTTGGC |
| | sequence | | |
| | 1904 SPN_A | 28 | CCTGATGAGTGCACAATGTAATTATCAAAATACACCAC |
| | | | GTTGGCCGAAACGTGGTGAAAGG |
| | 1904 SPN_B | 29 | CCAAACCTGATGAGTGCACAATGTAATTATCAAAATAC |
| | | | ACCACGTTGGCCGAAACGTGGTGACCAAGG |
| | 1904 SPN_C | 30 | CCAAAUUTGATGAGTGCACAATGTAATTATCAAAATAC |
| | | | ACCACGTTGGCCGAAACGTGGTGAAACCAUGG |
| | 1904 SPN_D | 31 | UAUAAUUTGATGAGTGCACAATGTAATTATCAAAATA |
| | | | CACCACGTTGGCCGAAACGTGGTGAAAUCUCUA |
| | 1904 SPN_E | 32 | UAUGGUUTGATGAGTGCACAATGTAATTATCAAAATA |
| | | | CACCACGTTGGCCGAAACGTGGTGAAAUCAAUA |
| | 1904 SPN_F | 33 | UGUUTGATGAGTGCACAATGTAATTATCAAAATACACC |
| | | | ACGTTGGCCGAAACGTGGTGAAAUCAAACA |
| | 1 RiboSPN_full | 34 | TAATACGACTCACTATAGGCGTAGCCTGATGAGCCACA |
| | sequence | | TCGTGCAATGCCCGAAACATACCACGTAGACGAAACG |
| | | | TGGTGAAAGCCACGTAGCTGCGCC |
| | 1 SPN_core | 35 | CCACATCGTGCAATGCCCGAAACATACCACGTAGA |
| | sequence | | |
| | 1 SPN_A | 36 | CTGAGCCACATCGTGCAATGCCCGAAACATACCACGTA |
| | | | GA CUCAG |
| | 1 SPN_B | 37 | GCCACATCGTGCAATGCCCGAAACATACCACGTAGA |
| | | | GGC |
| | 1 SPN_C | 38 | CCACATCGTGCAATGCCCGAAACATACCACGTAGA |
| | 1 SPN_D | 39 | GUCCAAACCACATCGTGCAATGCCCGAAACATACCAC |
| | | | GTAGAUGGAAAC |
| | 1 SPN_E | 40 | CGCAAACCACATCGTGCAATGCCCGAAACATACCACGT |
| | | | AGAAAGCG |
| | 1 SPN_F | 41 | CCACATCGTGCAATGCCCGAAACATACCACGTAGAUG |
| | | | UGG |
| | 1 SPN_G | 42 | CCACATCGTGCAATGCCCGAAACATACCACGTAGA |
| | | | CAAGG |
| | 28 | 43 | TAATACGACTCACTATAGGCGTAGCCTGATGAGCTATG |
| | RiboSPN_full | | CAGTGATGATTAAAGATACCACCACGTGAGCGAAACG |
| | sequence | | TGGTGAAAGCCACGTAGCTGCGCC |
| | 28 SPN_core | 44 | CTATGCAGTGATGATTAAAGATACCACCACGTGAG |
| | sequence | | |
| | 28 SPN_A | 45 | GATGAGCTATGCAGTGATGATTAAAGATACCACCACGT |
| | | | GAGCGAAACGTGGTGA |
| | 28 SPN_B | 46 | GCTATGCAGTGATGATTAAAGATACCACCACGTGAGCG |
| | | | AAACGTGGTGAAAGC |
| | 28 SPN_C | 47 | ACTATGCAGTGATGATTAAAGATACCACCACGTGAGCG |
| | | | AAACGTGGTGAAAGU |
| | 28 SPN_D | 48 | UACTATGCAGTGATGATTAAAGATACCACCACGTGAGC |
| | | | GAAACGTGGTGAGUA |
| | 28 SPN_E | 49 | GCTATGCAGTGATGATTAAAGATACCACCACGTGAG |
| | | | CGAAACGTGGTCCGC |
| | 28 SPN_F | 50 | GGUUGATGAGCTATGCAGTGATGATTAAAGATACCAC |
| | | | CACGTGAG CGAAACGTGGTGAAACC |
| | 28 SPN_G | 51 | UUGGATGAGCTATGCAGTGATGATTAAAGATACCACC |
| | | | ACGTGAGCGAAACGTGGTGACAA |
| | 28 SPN_H | 52 | UCGGATGAGCTATGCAGTGATGATTAAAGATACCACC |
| | | | ACGTGAGCGAAACGTGGTGAAACGA |
| | 28 SPN_I | 53 | AUGATGAGCTATGCAGTGATGATTAAAGATACCACCA |
| | | | CGTGAGCGAAACGTGGTGAAACCAU |
| | 28 SPN_J | 54 | AUGGAGCTATGCAGTGATGATTAAAGATACCACCACG |
| | | | TGAGCGAAACGTGGTGA AACAU |
| | 28 SPN_K | 55 | GUAAACTATGCAGTGATGATTAAAGATACCACCACGT |
| | | | GAGCGAAACGTGGTGAAUGC |
| Peanut | 2047 | 56 | TAATACGACTCACTATAGGCGTAGCCTGATGAGCAAAT |
| | RiboSPN_full | | AGTTACAAACACCACGTAGCGAAACGTGGTGAAAGCC |
| | sequence | | ACGTAGCTGCGCC |
| | 2047 SPN_core | 57 | CAAATAGTTACAAACACCACGTAG |
| | sequence | | |
| | 2047 SPN_A | 58 | UUCGCAAATAGTTACAAACACCACGTAGCGAA |
| | 2047 SPN_B | 59 | AUCGCAAATAGTTACAAACACCACGTAGCGAU |
| | 1981 | 60 | TAATACGACTCACTATAGGCGTAGCCTGATGAGCCCAA |
| | Ribo SPN_full | | CTGTACAGTACACCACGTAGCGAAACGTGGTGAAAGC |
| | sequence | | CACGTAGCTGCGCC |
| | 1981 SPN_core | 61 | CCCAACTGTACAGTACACCACGTAG |
| | sequence | | |
| | 1981 SPN_A | 62 | GAGCCCAACTGTACAGTACACCACGTAGCGAAACGTG |
| | | | GTGAAACUC |
| | 1981 SPN_B | 63 | UAUCCCAACTGTACAGTACACCACGTAGCGAAACGTG |
| | | | GTGAAAGGAAGAUA |
| | 2108 | 64 | TAATACGACTCACTATAGGCGTAGCCTGATGAGCACAC |
| | RiboSPN_full | | ACACATTCCACCACGTCACGCGAAACGTGGTGAAAGC |
| | sequence | | CACGTAGCTGCGCC |
| | 2108-SPN_core | 65 | CACACACACATTCCACCACGTCACG |
| | sequence | | |
| | 2108-SPN_A | 66 | CTGATGAGCACACACACATTCCACCACGTCACG |
| | 2108 SPN_B | 67 | GAUGATGAGCACACACACATTCCACCACGTCACG |
| | 2108 SPN_C | 68 | UGAUGATGAGCACACACACATTCCACCACGTCACGA |
| | 2108 SPN_D | 69 | UUAUUGATGAG CACACACACATTCCACCACGTCACG |
| | | | UAA |
| | 1785 | 70 | TAATACGACTCACTATAGGCGTAGCCTGATGAGCACAC |
| | RiboSPN_full | | GTTACCACACCACGTTGACGCGAAACGTGGTGAAAGC |
| | sequence | | CACGTAGCTGCGCC |
| | 1785 SPN_core | 71 | CACACGTTACCACACCACGTTGACG |
| | sequence | | |
| | 1785 SPN_A | 72 | AUCTGATGAG CACACGTTACCACACCACGTTGACG |
| | | | CGAAACGAGAU |
| | 1785 SPN_B | 73 | TAUUGAGCACACGTTACCACACCACGTTGACG |
| | | | CGAAUA |
| | 1785 SPN_C | 74 | TGAUUGAGCACACGTTACCACACCACGTTGACG |
| | | | CGAAUCA |
| | 1785 SPN_D | 75 | GUUGAGCACACGTTACCACACCACGTTGACG CGAA |
| | 1785 SPN_E | 76 | AUUGAGCACACGTTACCACACCACGTTGACG CGAU |
| | 1 RiboSPN_full | 77 | TAATACGACTCACTATAGGCGTAGCCTGATGAGCGTGC |
| | sequence | | CCGAAACACACACCACGATGCGAAACGTGGTGAAAGC |
| | | | CACGTAGCTGCGCC |
| | 1 SPN_core | 78 | CGTGCCCGAAACACACACCACGATG |
| | sequence | | |
| | 1 SPN_A | 79 | UUGAGCGTGCCCGAAACACACACCACGATGCGAAACG |
| | | | UCAA |
| | 1 SPN_B | 80 | UUAGCGTGCCCGAAACACACACCACGATGCGAAACGC |
| | | | UAA |
| | 1 SPN_C | 81 | GAUAGACGTGCCCGAAACACACACCACGATGCGAAAU |
| | | | GUC |
| | 1 SPN_D | 82 | UGAUAGACGTGCCCGAAACACACACCACGATGCGAAA |
| | | | UGUCA |
| | 7 RiboSPN_full | 83 | TAATACGACTCACTATAGGCGTAGCCTGATGAGCTCAC |
| | sequence | | CACATACCATGTACCACGTGCGAAACGTGGTGAAAGC |
| | | | CACGTAGCTGCGCC |
| | 7 SPN_core | 84 | CTCACCACATACCATGTACCACGTG |
| | Sequence | | |
| | 7 SPN_A | 85 | CCTGATGAGCTCACCACATACCATGTACCACGTGCGAA |
| | | | ACGTGGTGAAGC |
| | 7 SPN_B | 86 | GAG CTCACCACATACCATGTACCACGTG CGAAUGA |
| | 7 SPN_C | 87 | CTCACCACATACCATGTACCACG TGCGAAUGA |
| | 7 SPN_D | 88 | GCUCCTGATGAG CTCACCACATACCATGTACCACGTG |
| | | | CGAAACGTGGTGAAGC |
| | 7 SPN_E | 89 | CAUGATGAGCTCACCACATACCATGTACCACGTGCGAA |
| | | | CAUG |
| | 7 SPN_F | 90 | UCAUACGAGCTCACCACATACCATGTACCACGTGCGA |
| | | | AUGA |
| | 7 SPN_G | 91 | UCAUACGAGCTCACCACATACCATGTACCACGTGCGA |
| | | | AUGA |
| | 7 SPN_H | 92 | GCUCCTGATGAG CTCACCACATACCATGTACCACGTG |
| | | | CGAAACGTGGTGAAGC |
| | 7 SPN_I | 93 | CCTGATGAGCTCACCACATACCATGTACCACGTGCGAA |
| | | | ACGTGGTGAAGC |
| | 7 SPN_J | 94 | CAUGATGAGCTCACCACATACCATGTACCACGTGCGAA |
| | | | CAUG |
| | 7 SPN_K | 95 | UCAUACGAGCTCACCACATACCATGTACCACGTGCGA |
| | | | AUGA |
| | 7 SPN_L | 96 | UCAUACGAGCTCACCACATACCATGTACCACGTGCGA |
| | | | AUGA |
| | 35 | 97 | TAATACGACTCACTATAGGCGTAGCCTGATGAGTTCAC |
| | RiboSPN_full | | TGGCTGCACCCACCACCGCGTTCCACGAAACGTGGTGA |
| | sequence | | AAGCCACGTAGCTGCGCC |
| Milk | 35 SPN_core | 98 | TTCACTGGCTGCACCCACCACCGCGTTCCA |
| | sequence | | |
| | 35 SPN_A | 99 | GATGAGTTCACTGGCTGCACCCACCACCGCGTTCCACG |
| | | | AAACGTGGTGAAAGCCA |
| | 35 SPN_B | 100 | CGUCCTGATGAGTTCACTGGCTGCACCCACCACCGCGT |
| | | | TCCACGAAACGTGGTGAAAGCCAACG |
| | 35 SPN_C | 101 | AGCUCCTGATGAGTTCACTGGCTGCACCCACCACCGCG |
| | | | TTCCACGAAACGTGGTGAAAGCCAAGCU |
| | 35 SPN_D | 102 | AUCUCCTGATGAGTTCACTGGCTGCACCCACCACCGCG |
| | | | TTCCACGAAACGTGGTGAAAG |
| | 35 SPN_E | 103 | GCUACCTGATGAGTTCACTGGCTGCACCCACCACCGCG |
| | | | TTCCACGAAACGTGGTGAAAGCCAUAGC |
| | 35 SPN_F | 104 | CCACCTGATGAGTTCACTGGCTGCACCCACCACCGCGT |
| | | | TCCACGAAACGTGGTGAAAGCCAAAGG |
| | 35 SPN_G | 105 | UACCTGATGAGTTCACTGGCTGCACCCACCACCGCGTT |
| | | | CCACGAAACGTGGTGAAAGCCAGGUA |
| | 35 SPN_H | 106 | AUACCTGATGAGTTCACTGGCTGCACCCACCACCGCGT |
| | | | TCCACGAAACGTGGTGAAAGCCAGGUAU |
| | 35 SPN_I | 107 | CUACCTGATGAGTTCACTGGCTGCACCCACCACCGCGT |
| | | | TCCACGAAACGTGGTGAAAGCCAAAUAG |
| | 35 SPN_J | 108 | UACCTGATGAGTTCACTGGCTGCACCCACCACCGCGTT |
| | | | CCACGAAACGTGGTGAAAGCCAAAGUA |
| | 45 | 109 | TAATACGACTCACTATAGGCGTAGCCTGATGAGCATCC |
| | RiboSPN_full | | ACGGTGACGCTAATCCCACGTTCGACGAAACGTGGTG |
| | sequence | | AAAGCCACGTAGCTGCGCC |
| | 45 SPN_core | 110 | CATCCACGGTGACGCTAATCCCACGTTCGA |
| | sequence | | |
| | 45 SPN_A | 111 | CCTGATGAG |
| | | | CATCCACGGTGACGCTAATCCCACGTTCGA CGAAAGG |
| | 45 SPN_B | 112 | AGACCTGATGAGCATCCACGGTGACGCTAATCCCACGT |
| | | | TCGACGAAUCU |
| | 45 SPN_C | 113 | AGCCTGATGAGCATCCACGGTGACGCTAATCCCACGTT |
| | | | CGACGAAGCU |
| | 45 SPN_D | 114 | UAUCCTGATGAGCATCCACGGTGACGCTAATCCCACGT |
| | | | TCGACGGAAUA |
| | 45 SPN_E | 115 | GUCGATGAG |
| | | | CATCCACGGTGACGCTAATCCCACGTTCGA GAC |
| | 45 SPN_F | 116 | UCGATGAG |
| | | | CATCCACGGTGACGCTAATCCCACGTTCGA GA |
| | 45 SPN_G | 117 | UCGATGAG |
| | | | CATCCACGGTGACGCTAATCCCACGTTCGA G |
| | 74 | 118 | TAATACGACTCACTATAGGCGTAGCCTGATGAGACAAT |
| | RiboSPN_full | | GCAGATGCGCCCACCACGGATCACTCGAAACGTGGTG |
| | sequence | | AAAGCCACGTAGCTGCGCC |
| | 74 SPN_core | 119 | ACAATGCAGATGCGCCCACCACGGATCACT |
| | sequence | | |
| | 74 SPN_A | 120 | CCTGATGAG |
| | | | ACAATGCAGATGCGCCCACCACGGATCACT |
| | | | CGAAACGTGGTGAAAGCCA |
| | 74 SPN_B | 121 | GAG ACAATGCAGATGCGCCCACCACGGATCACT |
| | 74 SPN_C | 122 | GAG ACAATGCAGATGCGCCCACCACGGATCACT C |
| | 74 SPN_D | 123 | GAC ACAATGCAGATGCGCCCACCACGGATCACT CGUC |
| | 74 SPN_E | 124 | CCTGATGAG |
| | | | ACAATGCAGATGCGCCCACCACGGATCACT |
| | | | CGAAACGTGGTGAAAGCCAAGG |
| | 16 | 125 | TAATACGACTCACTATAGGCGTAGCCTGATGAGCAACC |
| | RiboSPN_full | | AAGCACGCTGCATCACGTTTCATCGCGAAACGTGGTGA |
| | sequence | | AAGCCACGTAGCTGCGCC |
| | 16 SPN_core | 126 | CAACCAAGCACGCTGCATCACGTTTCATCG |
| | sequence | | |
| | 16 SPN_A | 127 | GAGCAACCAAGCACGCTGCATCACGTTTCATCGCGAAA |
| | | | CGTGGCUC |
| | 16 SPN_B | 128 | GUUCAACCAAGCACGCTGCATCACGTTTCATCGCGAAA |
| | | | CGTGGAAC |
| | 16 SPN_C | 129 | UUCAACCAAGCACGCTGCATCACGTTTCATCGCGAAAC |
| | | | GTUGAA |
| | 16 SPN_D | 130 | CGATGAG CAACCAAGCACGCTGCATCACGTTTCATCG |
| | 1 RiboSPN_full | 131 | TAATACGACTCACTATAGGCGTAGCCTGATGAGCTCAC |
| | sequence | | AGCCCGAAACACATCGCCACGTTCACGAAACGTGGTG |
| | | | AAAGCCACGTAGCTGCGCC |
| | 1 SPN_core | 132 | CTCACAGCCCGAAACACATCGCCACGTTCA |
| | sequence | | |
| | 1 SPN_A | 133 | TGAG CTCACAGCCCGAAACACATCGCCACGTTCA |
| | 1 SPN_B | 134 | CGAAATGAG |
| | | | CTCACAGCCCGAAACACATCGCCACGTTCA AAACG |
| | 1 SPN_C | 135 | UAUCG CTCACAGCCCGAAACACATCGCCACGTTCA |
| | | | CGAAACGTGGTGAAAGAUA |
| Fish | 301 | 136 | TAATACGACTCACTATAGGCGTAGCCTGATGAGCTCAA |
| | RiboSPN_full | | TACTACGTCAATTCACAGATGATAGACACCACGGACG |
| | sequence | | AAACGTGGTGAAAGCCACGTAGCTGCGCC |
| | 301 SPN_core | 137 | CTCAATACTACGTCAATTCACAGATGATAGACACCACG |
| | sequence | | GA |
| | 301 SPN_A | 138 | GAGCTCAATACTACGTCAATTCACAGATGATAGACACC |
| | | | ACGGACGAAACGTGGTGAAAG |
| | 301 SPN_B | 139 | GAGCTCAATACTACGTCAATTCACAGATGATAGACACC |
| | | | ACGGACGAAACGTGGTGACUC |
| | 301 SPN_C | 140 | GUAAGCTCAATACTACGTCAATTCACAGATGATAGACA |
| | | | CCACGGACGAAACGTGGTGACUAC |
| | 301 SPN_D | 141 | CUAAGCTCAATACTACGTCAATTCACAGATGATAGACA |
| | | | CCACGGACGAAACGTGGTGACUAG |
| | 301 SPN_E | 142 | CUGUCTCAATACTACGTCAATTCACAGATGATAGACAC |
| | | | CACGGACGAAACGTGGTGACAG |
| | 301 SPN_F | 143 | CUGACTCAATACTACGTCAATTCACAGATGATAGACAC |
| | | | CACGGACGAAACGTGGTGACAG |
| | 301 SPN_G | 144 | CGACTCAATACTACGTCAATTCACAGATGATAGACACC |
| | | | ACGGACGAAACGTGGTGUUUG |
| | 301 SPN_H | 145 | UCUGACTCAATACTACGTCAATTCACAGATGATAGACA |
| | | | CCACGGACGAAACGTGGTGAUAGA |
| | 301 SPN_I | 146 | AAAAGCTCAATACTACGTCAATTCACAGATGATAGACA |
| | | | CCACGGACGAAACGTGGTGACUUAU |
| | 301 SPN_J | 147 | GAAAGCTCAATACTACGTCAATTCACAGATGATAGACA |
| | | | CCACGGACGAAACGTGGTGACUUAC |
| | 301 SPN_K | 148 | CUCAGCTCAATACTACGTCAATTCACAGATGATAGACA |
| | | | CCACGGACGAAACGTGGTGACGAG |
| | 333 | 149 | TAATACGACTCACTATAGGCGTAGCCTGATGAGTCCAA |
| | RiboSPN_full | | CACCACGTAACGTACACTGCATGTGATTGGTGCAACGA |
| | sequence | | AACGTGGTGAAAGCCACGTAGCTGCGCC |
| | 333 SPN_core | 150 | TCCAACACCACGTAACGTACACTGCATGTGATTGGTGC |
| | sequence | | AA |
| | 333 SPN_A | 151 | CCTGATGAGTCCAACACCACGTAACGTACACTGCATGT |
| | | | GATTGGTGCAACGAAACGTGG |
| | 333 SPN_B | 152 | TAGCCTGATGAGTCCAACACCACGTAACGTACACTGCA |
| | | | TGTGATTGGTGCAACGAAACGTGGTGA |
| | 333 SPN_C | 153 | GATGAGTCCAACACCACGTAACGTACACTGCATGTGAT |
| | | | TGGTGCAACGAA |
| | 333 SPN_D | 154 | UUAAGATGAGTCCAACACCACGTAACGTACACTGCAT |
| | | | GTGATTGGTGCAACGAAUUAA |
| | 333 SPN_E | 155 | UGAAGATGAGTCCAACACCACGTAACGTACACTGCAT |
| | | | GTGATTGGTGCAACGAAUUCA |
| | 333 SPN_F | 156 | UCACGATGAGTCCAACACCACGTAACGTACACTGCATG |
| | | | TGATTGGTGCAACGAA GUGA |
| | 365 | 157 | TAATACGACTCACTATAGGCGTAGCCTGATGAGTGGCG |
| | RiboSPN_full | | CCGACTGATCAACTAGACATCACGTTAGCATTCCGCGA |
| | sequence | | AACGTGGTGAAAGCCACGTAGCTGCGCC |
| | 365 SPN_core | 158 | TGGCGCCGACTGATCAACTAGACATCACGTTAGCATTC |
| | sequence | | CG |
| | 365 SPN_A | 159 | CCTGATGAGTGGCGCCGACTGATCAACTAGACATCACG |
| | | | TTAGCATTCCGCGAAACGTGG |
| | 365 SPN_B | 160 | GATGAGTGGCGCCGACTGATCAACTAGACATCACGTTA |
| | | | GCATTCCGCGAAACG |
| | 365 SPN_C | 161 | UGTGAGTGGCGCCGACTGATCAACTAGACATCACGTTA |
| | | | GCATTCCGCGAAACG |
| | 365 SPN_D | 162 | CCTGATGAGTGGCGCCGACTGATCAACTAGACATCACG |
| | | | TTAGCATTCCGCGAAACGAGG |
| | 365 SPN_E | 163 | GATGAGTGGCGCCGACTGATCAACTAGACATCACGTTA |
| | | | GCATTCCGCGAAACGTGGTGAAAGCCA |
| | 365 SPN_F | 164 | CATGAGTGGCGCCGACTGATCAACTAGACATCACGTTA |
| | | | GCATTCCGCGAAACGTGGTGAAAGCAUG |
| | 365 SPN_G | 165 | TAGAGTGGCGCCGACTGATCAACTAGACATCACGTTAG |
| | | | CATTCCGCGAAACGTGGTGAAAGCUCUA |
| | 365 SPN_H | 166 | AGAG |
| | | | TGGCGCCGACTGATCAACTAGACATCACGTTAGCATTC |
| | | | CG CGAAACGTGGTGAAAGCUUU |
| | 38 | 167 | TAATACGACTCACTATAGGCGTAGCCTGATGAGCCAGC |
| | RiboSPN_full | | AACCAGGTTACCTCCCATCACGCTTCGTCTCAGGACGA |
| | sequence | | AACGTGGTGAAAGCCACGTAGCTGCGCC |
| | 38 SPN_core | 168 | CCAGCAACCAGGTTACCTCCCATCACGCTTCGTCTCAG |
| | sequence | | GA |
| | 38 SPN_A | 169 | CCTGATGAG |
| | | | CCAGCAACCAGGTTACCTCCCATCACGCTTCGTCTCAG |
| | | | GA CGAAACG |
| | 38 SPN_B | 170 | GATGAG |
| | | | CCAGCAACCAGGTTACCTCCCATCACGCTTCGTCTCAG |
| | | | GA CGAAACG |
| | 38 SPN_C | 171 | GATGAG |
| | | | CCAGCAACCAGGTTACCTCCCATCACGCTTCGTCTCAG |
| | | | GA CGAA |
| | 38 SPN_D | 172 | CAAGATGAG |
| | | | CCAGCAACCAGGTTACCTCCCATCACGCTTCGTCTCAG |
| | | | GA CGAAUUG |
| | 38 SPN_E | 173 | CAAGATGAG |
| | | | CCAGCAACCAGGTTACCTCCCATCACGCTTCGTCTCAG |
| | | | GA CGAUUUG |
| | 38 SPN_F | 174 | CCAAGATGAG |
| | | | CCAGCAACCAGGTTACCTCCCATCACGCTTCGTCTCAG |
| | | | GA CGACUUUGG |
| | 38 SPN_G | 175 | ACAAGATGAG |
| | | | CCAGCAACCAGGTTACCTCCCATCACGCTTCGTCTCAG |
| | | | GA CGACUUUGU |
| | 38 SPN_H | 176 | ACAGATGAG |
| | | | CCAGCAACCAGGTTACCTCCCATCACGCTTCGTCTCAG |
| | | | GA CGACUUUGU |
| | 1 RiboSPN_full | 177 | TAATACGACTCACTATAGGCGTAGCCTGATGAGCTGAC |
| | sequence | | ACCACAAACGATTATGACCACGTTATCGTACATAGCGA |
| | | | AACGTGGTGAAAGCCACGTAGCTGCGCC |
| | 1 SPN_core | 178 | CTGACACCACAAACGATTATGACCACGTTATCGTACAT |
| | sequence | | AG |
| | 1 SPN_A | 179 | TAGGCGTAGCCTGATGAGCTGACACCACAAACGATTAT |
| | | | GACCACGTTATCGTACATAGCGAA |
| | 1 SPN_B | 180 | GAGCTGACACCACAAACGATTATGACCACGTTATCGTA |
| | | | CATAGCGAAACGTGGTGAAAGCCA |
| | 1 SPN_C | 181 | AGAGCTGACACCACAAACGATTATGACCACGTTATCGT |
| | | | ACATAGCGAAACGTGGTGAAAGCCAU |
| | 1 SPN_D | 182 | UGAGCTGACACCACAAACGATTATGACCACGTTATCGT |
| | | | ACATAGCGAAACGTGGTGAAAGCCUCA |
| | 1 SPN_E | 183 | UGAAGCTGACACCACAAACGATTATGACCACGTTATCG |
| | | | TACATAGCGAAACGTGGTGAAAGCCGUCA |
| | 1 SPN_F | 184 | UGAAGCTGACACCACAAACGATTATGACCACGTTATCG |
| | | | TACATAGCGAAACGTGGCGUCA |
| | 1 SPN_G | 185 | AUGAAGCTGACACCACAAACGATTATGACCACGTTATC |
| | | | GTACATAGCGAAACGTGGCGUCAU |
| | 1 SPN_H | 186 | GUAACTGACACCACAAACGATTATGACCACGTTATCGT |
| | | | ACATAG CGAAACGTGGTGAAAGCCUAC |
| | 1 SPN_I | 187 | UGUAAGCTGACACCACAAACGATTATGACCACGTTATC |
| | | | GTACATAGCGAAACGTGGTGAAAGAGGCA |
| | 1 SPN_J | 188 | UGUAAAGCTGACACCACAAACGATTATGACCACGTTA |
| | | | TCGTACATAG CGAAACGTGGTGAAAGAGGCA |
| | 27 | 189 | TAATACGACTCACTATAGGCGTAGCCTGATGAGTAGGT |
| | RiboSPN_full | | CAAGTGCGCTAAAACACACCGCGTTAGTTCACCAACG |
| | sequence | | AAACGTGGTGAAAGCCACGTAGCTGCGCC |
| | 27 SPN_core | 190 | TAGGTCAAGTGCGCTAAAACACACCGCGTTAGTTCACC |
| | sequence | | AA |
| | 27 SPN_A | 191 | ATAGGTCAAGTGCGCTAAAACACACCGCGTTAGTTCAC |
| | | | CAA CGAAACGTGGTGAAAGAUAAU |
| | 27 SPN_B | 192 | GATAGGTCAAGTGCGCTAAAACACACCGCGTTAGTTCA |
| | | | CCAA CGAAACGTGGTGAAAGAUAAUC |
| Egg | 1013 | 193 | TAATACGACTCACTATAGGCGTAGCCTGATGAGGCCAC |
| | RiboSPN_full | | CTCACTGTGTTTTGTTGCACAACATAATATGATGACGT |
| | sequence | | GCCGAAACGTGGTGAAAGCCACGTAGCTGCGCC |
| | 1013 SPN_core | 194 | GGCCACCTCACTGTGTTTTGTTGCACAACATAATATGA |
| | sequence | | TGACGTGC |
| | 1013 SPN_A | 195 | GCACGAGGCCACCUCACUGUGUUUUGUUGCACAACAU |
| | | | AAUAUGAUGACGUGC |
| | 1013 SPN_B | 196 | GCACGUAGGCCACCUCACUGUGUUUUGUUGCACAACA |
| | | | UAAUAUGAUGACGUGC |
| | 1013 SPN_C | 197 | GCACGUAUGGCCACCUCACUGUGUUUUGUUGCACAAC |
| | | | AUAAUAUGAUGACGUGC |
| | 1013 SPN_D | 198 | GCACCUGGGCCACCUCACUGUGUUUUGUUGCACAACA |
| | | | UAAUAUGAUGACGUGC |
| | 1013 SPN_E | 199 | GCACGGCCACCUCACUGUGUUUUGUUGCACAACAUAA |
| | | | UAUGAUGACGUGC |
| | 1013 SPN_F | 200 | TTGGCCACCUCACUGUGUUUUGUUGCACAACAUAAUA |
| | | | UGAUGACGUGCCGAA |
| | 1013 SPN_G | 201 | GCCACCUCACUGUGUUUUGUUGCACAACAUAAUAUG |
| | | | AUGACGUGCGGUGGC |
| | 1013 SPN_H | 202 | GCACGCCACCUCACUGUGUUUUGUUGCACAACAUAAU |
| | | | AUGAUGACGUGC |
| | 851 | 203 | TAATACGACTCACTATAGGCGTAGCCTGATGAGGCGTT |
| | RiboSPN_full | | CCCCACCGTTGCCCACGCTTAACTGGACAAAGATGGGC |
| | sequence | | CCCGAAACGTGGTGAAAGCCACGTAGCTGCGCC |
| | 851 SPN_core | 204 | GCGTTCCCCACCGTTGCCCACGCTTAACTGGACAAAGA |
| | sequence | | TGGGCCC |
| | 851 SPN_A | 205 | GCGTTCCCCACCGTTGCCCACGCTTAACTGGACAAAGA |
| | | | TGGGCCC AACGC |
| | 851 SPN_B | 206 | GGGCCCAGCGTTCCCCACCGTTGCCCACGCTTAACTGG |
| | | | ACAAAGATGGGCCC |
| | 851 SPN_C | 207 | ACCGCGTTCCCCACCGTTGCCCACGCTTAACTGGACAA |
| | | | AGATGGGCCCAGGA |
| | 851 SPN_D | 208 | TTGGGCGTTCCCCACCGTTGCCCACGCTTAACTGGACA |
| | | | AAGATGGGCCCGA |
| | 851 SPN_E | 209 | TAGGGCGTTCCCCACCGTTGCCCACGCTTAACTGGACA |
| | | | AAGATGGGCCCGAA |
| | 851 SPN_F | 210 | TGGCGTTCCCCACCGTTGCCCACGCTTAACTGGACAAA |
| | | | GATGGGCCCA |
| | 851 SPN_G | 211 | UGCGTTCCCCACCGTTGCCCACGCTTAACTGGACAAAG |
| | | | ATGGGCCCUGUG |
| | 851 SPN_H | 212 | TTGGGCGTTCCCCACCGTTGCCCACGCTTAACTGGACA |
| | | | AAGATGGGCCCGGAA |
| | 851 SPN_I | 213 | TTCGCGTTCCCCACCGTTGCCCACGCTTAACTGGACAA |
| | | | AGATGGGCCUGAG |
| | 505 | 214 | TAATACGACTCACTATAGGCGTAGCCTGATGAGTCTGT |
| | RiboSPN_full | | GCACATCACTCGACCTCTACGGCTGTATTGATCCTGCA |
| | sequence | | TACGAAACGTGGTGAAAGCCACGTAGCTGCGCC |
| | 505 SPN_core | 215 | TCTGTGCACATCACTCGACCTCTACGGCTGTATTGATCC |
| | sequence | | TGCATA |
| | 505 SPN_A | 216 | TCTGTGCACATCACTCGACCTCTACGGCTGTATTGATCC |
| | | | TGCATA GUGCACAGA |
| | 505 SPN_B | 217 | CCUAUGCTCTGTGCACATCACTCGACCTCTACGGCTGT |
| | | | ATTGATCCTGCATAGG |
| | 505 SPN_C | 218 | GCUAGAGTGCACATCACTCGACCTCTACGGCTGTATTG |
| | | | ATCCTGCATA AGAGC |
| | 780 | 219 | TAATACGACTCACTATAGGCGTAGCCTGATGAGCGTCC |
| | RiboSPN_full | | AACGTTCGATCAGAACCGCGTTCAGGCTGATGATTGTA |
| | sequence | | CGCGAAACGTGGTGAAAGCCACGTAGCTGCGCC |
| | 780 SPN_core | 220 | CGTCCAACGTTCGATCAGAACCGCGTTCAGGCTGATGA |
| | sequence | | TTGTACG |
| | 780 SPN_A | 221 | CGTCCAACGTTCGATCAGAACCGCGTTCAGGCTGATGA |
| | | | TTGTACGUUCCACG |
| | 780 SPN_B | 222 | GCUUACGTCCAACGTTCGATCAGAACCGCGTTCAGGCT |
| | | | GATGATTGTACGUAAGC |
| | 780 SPN_C | 223 | TGATGAGCGTCCAACGTTCGATCAGAACCGCGTTCAGG |
| | | | CTGATGATTGTACGCGAAACG |
| | 780 SPN_D | 224 | CGTGATGAGCGTCCAACGTTCGATCAGAACCGCGTTCA |
| | | | GGCTGATGATTGTACGCGACG |
| | 780 SPN_E | 225 | CAAGTGATGAGCGTCCAACGTTCGATCAGAACCGCGTT |
| | | | CAGGCTGATGATTGTACGCGACTTG |
| | 780 SPN_F | 226 | AUAGTGATGAGCGTCCAACGTTCGATCAGAACCGCGTT |
| | | | CAGGCTGATGATTGTACGCGACTAT |
| | 1 RiboSPN_full | 227 | TAATACGACTCACTATAGGCGTAGCCTGATGAGCATCA |
| | sequence | | GTGCGTTCTGCCTTTGCAACCACACAACACACCGTATG |
| | | | AGCGAAACGTGGTGAAAGCCACGTAGCTGCGCC |
| | 1 SPN_core | 228 | CATCAGTGCGTTCTGCCTTTGCAACCACACAACACACC |
| | sequence | | GTATGAG |
| | 1 SPN_A | 229 | CUCAUACATCAGTGCGTTCTGCCTTTGCAACCACACAA |
| | | | CACACCGTATGAG |
| | 1 SPN_B | 230 | GCUUACATCAGTGCGTTCTGCCTTTGCAACCACACAAC |
| | | | ACACCGTATGAG UAAGC |
| | 1 SPN_C | 231 | CCTGATGAGCATCAGTGCGTTCTGCCTTTGCAACCACA |
| | | | CAACACACCGTATGAG CGAAACG |
| | 1 SPN_D | 232 | GCAGCATCAGTGCGTTCTGCCTTTGCAACCACACAACA |
| | | | CACCGTATGAGCGC |
| | 1 SPN_E | 233 | TTGAGCATCAGTGCGTTCTGCCTTTGCAACCACACAAC |
| | | | ACACCGTATGAGCGAA |
| | 1 SPN_F | 234 | TGCAGCATCAGTGCGTTCTGCCTTTGCAACCACACAAC |
| | | | ACACCGTATGAGCGCG |
| | 1 SPN_G | 235 | CGCAGCATCAGTGCGTTCTGCCTTTGCAACCACACAAC |
| | | | ACACCGTATGAGCGCG |
| | 17 | 236 | TAATACGACTCACTATAGGCGTAGCCTGATGAGCCAAC |
| | RiboSPN_full | | TGTGCACACTGTTCGCTTATCGAGCTGTGTACCTCCAT |
| | sequence | | AGCGAAACGTGGTGAAAGCCACGTAGCTGCGCC |
| | 17 SPN_core | 237 | CCAACTGTGCACACTGTTCGCTTATCGAGCTGTGTACC |
| | sequence | | TCCATAG |
| | 17 SPN_A | 238 | CCTGATGAGCCAACTGTGCACACTGTTCGCTTATCGAG |
| | | | CTGTGTACCTCCATAGCGAAACGTGG |
| | 17 SPN_B | 239 | ACUCCTGATGAGCCAACTGTGCACACTGTTCGCTTATC |
| | | | GAGCTGTGTACCTCCATAGCGAAACGAGGCAT |
| | 17 SPN_C | 240 | ATGCCTGATGAGCCAACTGTGCACACTGTTCGCTTATC |
| | | | GAGCTGTGTACCTCCATAGCGAAACGTGGTGAA |
| | 17 SPN_D | 241 | AACCTGATGAGCCAACTGTGCACACTGTTCGCTTATCG |
| | | | AGCTGTGTACCTCCATAG CGAAACGTGGCA |
| | 17 SPN_E | 242 | AACTGATGAGCCAACTGTGCACACTGTTCGCTTATCGA |
| | | | GCTGTGTACCTCCATAG CGAAACAGUU |
| | 17 SPN_F | 243 | CATGCCTGATGAGCCAACTGTGCACACTGTTCGCTTAT |
| | | | CGAGCTGTGTACCTCCATAG CGAAACGTGGTGAAGUG |
| | 17 SPN_G | 244 | CCAACTGTGCACACTGTTCGCTTATCGAGCTGTGTACC |
| | | | TCCATAG GUUGG |
| | 17 SPN_H | 245 | CUAUGGCCAACTGTGCACACTGTTCGCTTATCGAGCTG |
| | | | TGTACCTCCATAG |
| Gluten | 457 | 246 | TAATACGACTCACTATAGGCGTAGCCTGATGAGCTTGG |
| | RiboSPN_full | | TCACCTTTCCTGACATTAACACAGGCGAAACGTGGTGA |
| | sequence | | AAGCCACGTAGCTGCGCC |
| | 457 SPN_core | 247 | CTTGGTCACCTTTCCTGACATTAACACAGG |
| | sequence | | |
| | 457 SPN_A | 248 | CTTGGTCACCTTTCCTGACATTAACACAGGCCAAG |
| | 457 SPN_B | 249 | CCUGUCTTGGTCACCTTTCCTGACATTAACACAGG |
| | 491 | 250 | TAATACGACTCACTATAGGCGTAGCCTGATGAGTTTTC |
| | RiboSPN_full | | CCGATACGGCTACGAATTGCGACAACGAAACGTGGTG |
| | sequence | | AAAGCCACGTAGCTGCGCC |
| | 491 SPN_core | 251 | TTTTCCCGATACGGCTACGAATTGCGACAA |
| | sequence | | |
| | 491 SPN_A | 252 | CC TTTTCCCGATACGGCTACGAATTGCGACAA AAGG |
| | 491 SPN_B | 253 | GCUUA TTTTCCCGATACGGCTACGAATTGCGACAA |
| | | | UAAGC |
| | 578 | 254 | TAATACGACTCACTATAGGCGTAGCCTGATGAGGCACC |
| | RiboSPN_full | | AATTTTACCGATTTTGGTGGACAGCCGAAACGTGGTGA |
| | sequence | | AAGCCACGTAGCTGCGCC |
| | 578 SPN_core | 255 | GCACCAATTTTACCGATTTTGGTGGACAGC |
| | sequence | | |
| | 578 SPN_A | 256 | GCUGUCC GCACCAATTTTACCGATTTTGGTGGACAGC |
| | 578 SPN_B | 257 | GCACCAATTTTACCGATTTTGGTGGACAGC UUGGUGC |
| | 1514 | 258 | TAATACGACTCACTATAGGCGTAGCCTGATGAGCGTAC |
| | RiboSPN_full | | AACCCACCACCGTTGTCCACAAATGCGAAACGTGGTG |
| | sequence | | AAAGCCACGTAGCTGCGCC |
| | 1514 SPN_core | 259 | CGTACAACCCACCACCGTTGTCCACAAATG |
| | sequence | | |
| | 1514 SPN_A | 260 | CAUUUG CGTACAACCCACCACCGTTGTCCACAAATG |
| | 1514 SPN_B | 261 | CGTACAACCCACCACCGTTGTCCACAAATG UUGUACG |
| | 1 RiboSPN_full | 262 | TAATACGACTCACTATAGGCGTAGCCTGATGAGTGCGT |
| | sequence | | CAACGGCCGTCCCGAAACGTGAATACGAAACGTGGTG |
| | | | AAAGCCACGTAGCTGCGCC |
| | 1 SPN_core | 263 | TGCGTCAACGGCCGTCCCGAAACGTGAATA |
| | sequence | | |
| | 1 SPN_A | 264 | UAUUCA TGCGTCAACGGCCGTCCCGAAACGTGAATA |
| | 1 SPN_B | 265 | GCUUA TGCGTCAACGGCCGTCCCGAAACGTGAATA |
| | | | UAAGC |
| | 35 | 266 | TAATACGACTCACTATAGGCGTAGCCTGATGAGGTTAC |
| | RiboSPN_full | | CCCGAAACGGCCCTAACTGCATCAGCGAAACGTGGTG |
| | sequence | | AAAGCCACGTAGCTGCGCC |
| | 35 SPN_core | 267 | GTTACCCCGAAACGGCCCTAACTGCATCAG |
| | sequence | | |
| | 35 SPN_A | 268 | CUGAUGC GTTACCCCGAAACGGCCCTAACTGCATCAG |
| | 35 SPN_B | 269 | GTTACCCCGAAACGGCCCTAACTGCATCAG GGGUAAC |
| Soy | 1 RiboSPN_full | 270 | TAATACGACTCACTATAGGCGTAGCCTGATGAGCCGCA |
| | sequence | | TCACCACCCAAACCACCGTTCGAAACGTGGTGAAAGC |
| | | | CACGTAGCTGCGCC |
| | 1 SPN_core | 271 | CCGCATCACCACCCAAACCACCGTT |
| | sequence | | |
| | 1 SPN_A | 272 | GCCTGATGAG CCGCATCACCACCCAAACCACCG |
| | | | TTCGAAACGAGGC |
| | 1 SPN_B | 273 | TTCCAATGATGAG CCGCATCACCACCCAAACCACCG |
| | | | TTCGAAACGAGGAA |
| | 1 SPN_C | 274 | TATACAATGATGAG CCGCATCACCACCCAAACCACCG |
| | | | TTCGAAACGAGTAAG |
| | 1 SPN_D | 275 | UUCCTGATGAG CCGCATCACCACCCAAACCACCGTT |
| | | | CGAAAUAGGAA |
| | 1 SPN_E | 276 | TGGACAATGATGAG CCGCATCACCACCCAAACCACCG |
| | | | TTCGAAACGAGTCACA |
| | 1 SPN_F | 277 | TCCACAATGATGAG CCGCATCACCACCCAAACCACCG |
| | | | TTCGAAACGAGTGAGA |
| | 2 RiboSPN_full | 278 | TAATACGACTCACTATAGGCGTAGCCTGATGAGCCTGC |
| | sequence | | TCCATCCGCGCCAGCCTCACCGAAACGTGGTGAAAGC |
| | | | CACGTAGCTGCGCC |
| | 2 SPN_core | 279 | CCTGCTCCATCCGCGCCAGCCTCAC |
| | sequence | | |
| | 2 SPN_A | 280 | CCTGATGAGCCTGCTCCATCCGCGCCAGCCTCACCGAA |
| | | | ACG |
| | 2 SPN_B | 281 | TAGCCTGATGAGCCTGCTCCATCCGCGCCAGCCTCACC |
| | | | GAAACGTGGCTA |
| | 2 SPN_C | 282 | CTTAGCCTGATGAG |
| | | | CCTGCTCCATCCGCGCCAGCCTCAC CGTAAG |
| | 2 SPN_D | 283 | CTTAGGGTAGAGCCTGCTCCATCCGCGCCAGCCTCACC |
| | | | UTAAG |
| | 2 SPN_E | 284 | TTAGGGTAGAG CCTGCTCCATCCGCGCCAGCCTCAC |
| | | | CUTAA |
| | 2 SPN_F | 285 | GTTAGGGTAGAG CCTGCTCCATCCGCGCCAGCCTCAC |
| | | | CUAAU |
| | 2 SPN_G | 286 | TTAGGTAGAG CCTGCTCCATCCGCGCCAGCCTCAC |
| | | | CUAA |
| | 2 SPN_H | 287 | GCGTAGCCTGATGAG |
| | | | CCTGCTCCATCCGCGCCAGCCTCAC |
| | | | CGAAACGTGGTGAAAG |
| | 2 SPN_I | 288 | TAATACGACCCTGCTCCATCCGCGCCAGCCTCACCGAA |
| | | | ACGTGGTGAAAG |
| | 2 SPN_J | 289 | TCACTATAGCCTGCTCCATCCGCGCCAGCCTCACCGAA |
| | | | ACGTGGTGAAAG |
| | 2 SPN_K | 290 | CCACGTAGCTGCGCCCCTGCTCCATCCGCGCCAGCCTC |
| | | | AC CGAAACGTGGTGAAAG |
| | 2 SPN_L | 291 | CUUTCACCTGATGAG |
| | | | CCTGCTCCATCCGCGCCAGCCTCAC |
| | | | CGAAACGTGGTGAAAG |
| | 2 SPN_M | 292 | UAUTCACCTGATGAG |
| | | | CCTGCTCCATCCGCGCCAGCCTCAC |
| | | | CGAAACGTGGTGAAAG |
| | 2 SPN_N | 293 | UTCGATGAGCCTGCTCCATCCGCGCCAGCCTCACCGAA |
| | | | CAG |
| | 2 SPN_O | 294 | CAUTCGATGAGCCTGCTCCATCCGCGCCAGCCTCACCG |
| | | | AACUG |
| | 3 RiboSPN_full | 295 | TAATACGACTCACTATAGGCGTAGCCTGATGAGCCAAT |
| | sequence | | CTCCTGCCCACGCCGTTCCACGAAACGTGGTGAAAGCC |
| | | | ACGTAGCTGCGCC |
| | 3 SPN_core | 296 | CCAATCTCCTGCCCACGCCGTTCCA |
| | sequence | | |
| | 3 SPN_A | 297 | TAGGCGTAGCCTGATGAGCCAATCTCCTGCCCACGCCG |
| | | | TTCCACGAAACGTGGTGAAAG |
| | 3 SPN_B | 298 | TAGGCGTAGCCTGATGAGCCAATCTCCTGCCCACGCCG |
| | | | TTCCACGAA |
| | 3 SPN_C | 299 | TAGGCGTAGCCTGATGAGCCAATCTCCTGCCCACGCCG |
| | | | TTCCACCUACGCCUA |
| | 3 SPN_D | 300 | GCGTAGCCTGATGAG |
| | | | CCAATCTCCTGCCCACGCCGTTCCA CCUACGC |
| | 3 SPN_E | 301 | UCGTAGCCTGATGAG |
| | | | CCAATCTCCTGCCCACGCCGTTCCA CCUACCA |
| | 3 SPN_F | 302 | UGCTAGCCTGATGAG |
| | | | CCAATCTCCTGCCCACGCCGTTCCA CCUAGAA |
| | 3 SPN_G | 303 | CGATAGCCTGATGAG |
| | | | CCAATCTCCTGCCCACGCCGTTCCA CCUATAG |
| | 3 SPN_H | 304 | GUCCTAGCCTGATGAGCCAATCTCCTGCCCACGCCGTT |
| | | | CCACAUCUUGCUAGAAC |
| | 3 SPN_I | 305 | GATGAGCCAATCTCCTGCCCACGCCGTTCCACAAGCUC |
| | | | AUC |
| | 3 SPN_J | 306 | CCTGATGAGCCAATCTCCTGCCCACGCCGTTCCACAUC |
| | | | GAAAGG |
| | 4 RiboSPN_full | 307 | TAATACGACTCACTATAGGCGTAGCCTGATGAGCCAAT |
| | sequence | | CAAGGACCGCCTTCACCGCTCGAAACGTGGTGAAAGC |
| | | | CACGTAGCTGCGCC |
| | 4 SPN_core | 308 | CCAATCAAGGACCGCCTTCACCGCT |
| | sequence | | |
| | 4 SPN_A | 309 | CCTGATGAG CCAATCAAGGACCGCCTTCACCGCT |
| | | | CGAAACG |
| | 4 SPN_B | 310 | GATGAG CCAATCAAGGACCGCCTTCACCGCT CGAU |
| | 4 SPN_C | 311 | TGAG CCAATCAAGGACCGCCTTCACCGCT CG |
| | 4 SPN_D | 312 | GCACAG CCAATCAAGGACCGCCTTCACCGCT UCU |
| | 4 SPN_E | 313 | GCAACAG CCAATCAAGGACCGCCTTCACCGCT UUU |
| | 4 SPN_F | 314 | GACAG CCAATCAAGGACCGCCTTCACCGCT UUU |
| | 5 RiboSPN_full | 315 | TAATACGACTCACTATAGGCGTAGCCTGATGAGACTCT |
| | sequence | | CGCATCACCAGCCAACTCACCGAAACGTGGTGAAAGC |
| | | | CACGTAGCTGCGCC |
| | 5 SPN_core | 316 | ACTCTCGCATCACCAGCCAACTCAC |
| | sequence | | |
| | 5 SPN_A | 317 | TAGCCTGATGAG ACTCTCGCATCACCAGCCAACTCAC |
| | | | CGAAACGTGGTGAAAG |
| | 5 SPN_B | 318 | TAGCCTGATGAG ACTCTCGCATCACCAGCCAACTCAC |
| | | | CGAAACGTGGTGGCUA |
| | 5 SPN_C | 319 | TAGCCAGATGAG ACTCTCGCATCACCAGCCAACTCAC |
| | | | CGAAACGTGGTGGCUA |
| | 5 SPN_D | 320 | TAGCCAGATGAG ACTCTCGCATCACCAGCCAACTCAC |
| | | | CGAACACAUCTGGCUA |
| | 5 SPN_E | 321 | TCGCCAGATGAG ACTCTCGCATCACCAGCCAACTCAC |
| | | | CGAAACGTGGTGGCGA |
| | 5 SPN_F | 322 | TCGCCAGATGAG ACTCTCGCATCACCAGCCAACTCAC |
| | | | CGAAACGTGGAGGCGA |
| | 5 SPN_G | 323 | TUGCCAGATGAG ACTCTCGCATCACCAGCCAACTCAC |
| | | | CGAAACGTGGAGGCGA |
| | 5 SPN_H | 324 | TUGCCAGATGAG ACTCTCGCATCACCAGCCAACTCAC |
| | | | CGAAACGTGGAGUUGCGA |
| | 5 SPN_I | 325 | TUGCCUUGATGAG |
| | | | ACTCTCGCATCACCAGCCAACTCAC |
| | | | CGAAACGTGGAGGCGA |
| | 5 SPN_J | 326 | TUGCCUAUGATGAG |
| | | | ACTCTCGCATCACCAGCCAACTCAC |
| | | | CGAAACGTGGAGGCGA |
| | 5 SPN_K | 327 | TUGCCUGATGAG ACTCTCGCATCACCAGCCAACTCAC |
| | | | CGAAACGAATGGAGGCGA |
| | 5 SPN_L | 328 | TUGCCUGATGAG ACTCTCGCATCACCAGCCAACTCAC |
| | | | CGAAACGGTGGAGGCGA |
| | 5 SPN_M | 329 | TUGCCUGATGAG ACTCTCGCATCACCAGCCAACTCAC |
| | | | CGAAACGTGGAGGCAT |
| | 5 SPN_N | 330 | TUGCCUGATGAG ACTCTCGCATCACCAGCCAACTCAC |
| | | | CGAAACGTGGAGGCAA |
| Crustacean | 1 RiboSPN_full | 331 | TAATACGACTCACTATAGGCGTAGCCTGATGAGCGGTA |
| | sequence | | CTCAGATTACAGAGTGACATCGAAACGTGGTGAAAGC |
| | | | CACGTAGCTGCGCC |
| | 1 SPN_core | 332 | CGGTACTCAGATTACAGAGTGACAT |
| | sequence | | |
| | 2 RiboSPN_full | 333 | TAATACGACTCACTATAGGCGTAGCCTGATGAGAGACA |
| | sequence | | CCACGGATCCGAACTGGAGCGAAACGTGGTGAAAGCC |
| | | | ACGTAGCTGCGCC |
| | 2 SPN_core | 334 | AGACACCACGGATCCGAACTGGAG |
| | sequence | | |
| | 3 RiboSPN_full | 335 | TAATACGACTCACTATAGGCGTAGCCTGATGAGCCTCG |
| | sequence | | CAAGATTGCATACGTTAGAACGAAACGTGGTGAAAGC |
| | | | CACGTAGCTGCGCC |
| | 3 SPN_core | 336 | CCTCGCAAGATTGCATACGTTAGAA |
| | sequence | | |
| | 4 RiboSPN_full | 337 | TAATACGACTCACTATAGGCGTAGCCTGATGAGCACGT |
| | sequence | | AGGAAACGACCTCTACGGAGCGAAACGTGGTGAAAGC |
| | | | CACGTAGCTGCGCC |
| | 4 SPN_core | 338 | CACGTAGGAAACGACCTCTACGGAG |
| | sequence | | |
| | 5 RiboSPN_full | 339 | TAATACGACTCACTATAGGCGTAGCCTGATGAGCCCGA |
| | sequence | | AACCACCACCGTTGTCCAATACGAAACGTGGTGAAAG |
| | | | CCACGTAGCTGCGCC |
| | 5 SPN_core | 340 | CCCGAAACCACCACCGTTGTCCAATA |
| | sequence | | |

In some embodiments, signaling polynucleotides of the present invention may be generated by modifying the original allergen binding aptamers disclosed in the literature. The parent sequence of each aptamer against a specific allergen is modified to comprise the shortest sequence without changing the binding specificity and affinity of the aptamer. Some exemplary signaling polynucleotides modified from known parent sequences are listed in Table 2.

TABLE 2
SPNs originated from literature sequences

| | | SEQ | |
|---|---|---|---|
| | | ID | |
| Allergen | SPN ID | NO. | Sequence (5′-3′) |
| Gluten | GLI4-parent | 341 | CCAGTCTCCCGTTTACCGCGCCTACACATGTCTGAATG |
| | sequence | | CC |
| | GLI4 SPN-1 | 342 | TAGTCTCCCGTTTACCGCGCCTACACATGTCTGAATG |
| | GLI4 SPN-2 | 343 | TAGTCTCCCGTTTACCGCGCCTACACATGTCTGAA |
| | GLI1-parent | 344 | CTAGGCGAAATATAGCTACAACTGTCTGAAGGCACCCA |
| | sequence | | AT |
| Egg | 2007-parent | 345 | ATCTACGAATTCATCAGGGCTAAAGAGTGCAGAGTTA |
| | sequence | | CTTAG |
| | 2007 SPN-1 | 346 | TACGAATTCATCAGGGCTAAAGAGTGCAGAGTTACT |
| | 2007 SPN-2 | 347 | AAT |
| | | | ATCTACGAATTCATCAGGGCTAAAGAGTGCAGAGTTA |
| | | | CTTAG CTC |
| | 2007 SPN-3 | 348 | ATCTACGAATTCATCAGGGCTAAAGAGTGCAGAGTTAC |
| | | | TTAG |
| | 2012-parent | 349 | GCAGCTAAGCAGGCGGCTCACAAAACCATTCGCATGC |
| | sequence | | GGC |
| | 2012 SPN-1 | 350 | GCTAAGCAGGCGGCTCACAAAACCATTCGCATGC |
| | 2012 SPN-2 | 351 | GCTAAGCAGGCGGCTCACAAAACCATTCGCATGC AA |
| Ellington | Parent sequence | 352 | GGUUGUGAAGAUUGGGAGCGUCGUGGCUAC |
| and Cox | | | |
| Peanut | ARAH1-parent | 353 | TCGCACATTCCGCTTCTACCGGGGGGGTCGAGCTGAGT |
| | sequence | | GGATGCGAATCTGTGGGTGGGCCGTAAGTCCGTGTGT |
| | | | GCGAA |

### Detection Agents

In accordance with the present invention, aptamer derived signaling polynucleotides (SPNs) can be used as detection agents in a variety of allergen detection assays, biosensors, detection systems and devices as disclosed in the prior art. In one example, the present SPNs may be used as surface bound affinity molecules that bind the surfaces of magnetic particles, or detector agents, or competitive binding agents which are reagents used in the system of identify the presence, absence and/or quantity of a molecule of interest in a sample as disclosed by Ayub et al in U.S. Patent Application Publication No.: 2014/0336083; in a microfluidic channel of the electrochemical sensor for detection of a target analyte by Ayub and Clinton in U.S. Pat. No. 9,207,245; the analyte detection kits by Ayub and Clinton in U.S. Pat. No. 9,207,244; in the sample analysis cartridge of the target analyte detection assays by Ayub and Clinton in U.S. Pat. Nos. 9,086,417; 9,034,168 and 9,052,275; the contents of each of which are incorporated by reference herein in their entirety.

In another example, the present SPNs may be used to coat magnetic particles to form functionalized magnetic particles which can capture a target analyte in a fluid sample when the sample flows through the capillary channel of a filtration device disclosed in U.S. Pat. Nos. 8,895,320 and 9,244,068 to Florescu; and in a magnetic particle based biosensor disclosed in U.S. patent application publication No.: 2013/0230913 to Florescu et al., and PCT patent application publication No.: WO2014/189624 to Florescu et al; the contents of each of which are incorporated by reference herein in their entirety. In addition, the present SPNs may also be linked to the sensing areas on the exposed surface of the integrated circuit to specifically capture target analyte-functionalized magnetic particle complexes for detection as disclosed in Florescu's biosensors.

In this context, the present SPNs may be conjugated magnetic particles for magnetic manipulations in a detection assay and method. The present SPNs coupled to magnetic particles may have nucleic acid sequences listed as SEQ ID NOs.: 1-353.

### Magnetic Beads/Magnetic Particles

Magnetic beads have several advantages that make them attractive candidates for use as signal transducers, including their biological inertness, physical stability, and the absence of competing magnetic signals in biological materials (Gijs et al., Chem Rev. 2010; Vol 110(3), 1518-1563).

Magnetic particles may be any particle materials that can be separated by magnetic forces. Magnetic particles for bioresearch may consist of one or more magnetic cores with a coating matrix of polymers, silica or hydroxylapatite with terminal functionalized groups. The magnetic core generally consists either of magnetite (Fe₃O₄) or maghemite (γ-Fe₂O₃) with superparamagnetic or ferromagnetic properties. For example, magnetic cores can be made with magnetic ferrites, such as cobalt ferrite or manganese ferrite. Such magnetic micro- or nanospheres can be separated easily and quickly by magnetic forces and can be used together with bioaffine ligands, e.g. antibodies or aptamers with a high affinity to the target.

In some embodiments, magnetic particles may be in different sizes. As used herein, the particle size (particle diameter) is given as hydrodynamic diameter, which includes the core diameter and two times the diameter of the cover matrix. In some examples, magnetic particles may be in a wide range of sizes, from 100 nm to 5 μm, having optimized parameters such as sedimentation rate, available binding sites, and magnetic volume.

As non-limiting examples, magnetic particles may be fluidMAG particles (which is hydrophilic), SiMAG particles (which are magnetic silica beads with superparamagnetic or ferromagnetic properties and possess either a highly porous or a non-porous silica surface), mHPA-particles (which are non-spherical with hydroxylapatite coated ferromagnetic particles with a diameter of 2 μm, consisting of calcium phosphate), ZeoliteMAG (which are magnetic zeolite particles, which consist of a superparamagnetic iron oxide core and a high-porous aluminosilicate matrix), beadMAG-particles (which are magnetic particles with a diameter of 1 μm, covered with a hydrophilic matrix of crosslinked starch with terminal cation-exchange phosphate groups), and magTosyl-magnetic beads or other appropriately derived magnetic beads for nucleic acid conjugations. In other embodiments, polystyrene magnetic particles may be used. The magnetic particles may also be sepharose magnetic microbeads and agarose magnetic microbeads.

In some embodiments, magnetic particles, in the absence of a magnetic field, may exhibit no net magnetization, but within a magnetic field, the magnetic moments of the bead align with the field, making the beads magnetic.

### Aptamer-Magnetic Particle Conjugates

Aptamers can be conjugated to magnetic particles by any method known in the art.

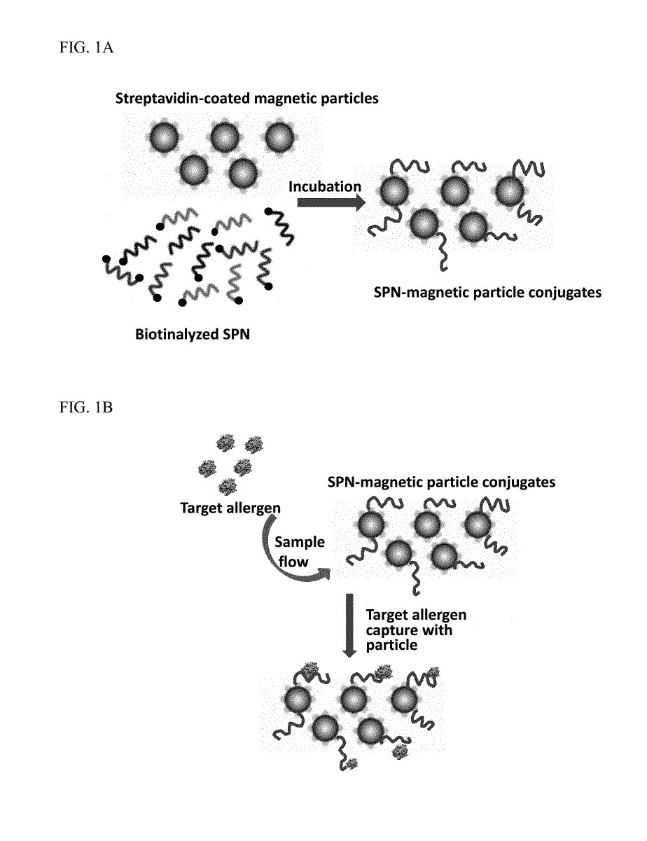

In one example, the present SPNs may be attached to magnetic particles using biotin-streptavidin system as shown in FIG. 1A. The biotin-streptavidin system is widely used in the art since the binding constant of biotin and streptavidin is 10¹⁵M−1 providing nearly covalent binding and fast binding kinetics. Biotin and molecule derived from biotin react with primary amines of proteins, nucleic acids and other molecules to form stable biotin conjugates. Biotinalyted aptamers can be coupled to streptavidin coated magnetic particles and the aptamer-magnetic particle conjugates may be separated from free biotinylated aptamers by magnet (FIG. 1A).

In another example, the present SPNs may be coupled to magnetic particles through EDC mediated coupling method. This coupling method lays covalent binding of amino-modified aptamer (SPNs) to carboxyl-functionalized magnetic particles.

In some aspects, acid treated magnetic particles containing hydroxyl (OH) groups on the surface can be used to conjugate ligands including aptamers as disclosed in U.S. Patent application publication No.: US2014/0206822, the content of which is incorporated herein by reference in its entirety.

In further another example, molecular spacers may be used to mediate the coupling between aptamers and magnetic particles. The method can avoid interaction between the solid surface and the aptamer conformation.

In other examples, magnetic particles may be coated with a short olionucleotide which comprises a short linker sequence (e.g., 5 nt in length) and a short sequence (e.g., 5 nt in length) complementary to either the 5′ terminus or the 3′ terminus of the SPN. The coated magnetic particles are attached to aptamers/SPNs through the complementary interaction. The conjugation to Aptamers/SPNs brings magnetic particles close to each other, forming aggregates. The aptamer magnetic particle aggegates may be separated in the presence of target analytes. The binding of target analyte to aptamer/SPN interrupts the interaction between magnetic particles and pulls the beads away from each other. The magnetic field changes before and after the target analyte binding can be detected and measured as the absence, presence and amount of the target analyte.

### Multiple-Aptamer Magnetic Particle Conjugates

SELEX methodology has a significant advantage since it is generally feasible to develop a panel of aptamers that can selectively recognize different parts of the same target. To take advantage of this property, a strategy to incorporate multiple aptamers into magnetic particles may be used for specific target analyte recognition. Utilizing multiple aptamer sequences also can make magnetic particles more widely applicable.

In some embodiments, the present SPNs may be conjugated with a fluorophore as a detection agent. The aptamer-magnetic conjugates and aptamer-fluorescent conjugates may be used in combination for binding, separation of analytes from a sample and fluorescence detection.

### Fluorescent Probed Signal Polynucleotides

In some embodiments, aptamer derived signal polynucleotides and/or aptamer magnetic particle conjugates may be labeled with a fluorescent marker (e.g., a fluorophore) as detection agents. In some examples, aptamers/SPNs are labeled with both a quencher and a fluorophore, the fluorescent intensity upon allergen binding to the aptamer is measured. Using fluorophore-quencher pairs, a FRET signal between fluorescein and quency (e.g. DABCYL moieties) is used to detect the presence of the target analyte (e.g. allergen). Such detection agents are disclosed in patent applications of the present inventors (e.g., PCT application publication NO.: 2015/066027; and U.S. Provisional Application Ser. No. 62/154,200; the contents of each of which are incorporated by reference in their entirety). In other examples, SPNs may be labeled with one or more fluorephore at either or both ends. The fluorescent polarization can be detected optically. Such detection agents can be found in patent application by the present inventors, U.S. Provisional Application Ser. No. 62/308,377; the contents of which are incorporated by reference herein in its entirety.

### Electroactive Reporter Labeled Signal Polynucleotides

In some embodiments, aptamer derived signal polynucleotides and/or aptamer magnetic particle conjugates may be labeled with an electroactive reporter (also called electrical signal transducer, or a redox indicator, or c conductive molecule) as detection agents. In this context, a SPN or an aptamer/SPN magnetic particle conjugate based sensor may be an electrical or electrochemical sensor, wherein the free SPN or aptamer-magnetic particle complex is covalently bonded to or chemisorbed on the surface of an electrode. The electron exchange between the target analyte that binds to the aptamer magnetic particle conjugates and the electrode may be transformed as amperometric and potentiometric signals to be detected.

In some embodiments, SPNs may be directly linked to or coated with the surface of an electrode, through either their 5′ termini or 3′ termini. In some aspects, a short nucleic acid linker may be used to link SPNs to the surface of an electrode. The short nucleic acid linker may contain 5 to 30 nucleotide residues. In particular, it may contain 5, 6, 7, 8, 9, 10, 11, 12, 13, 14, 15, 16, 17, 18, 19, 20, 21, 22, 23, 24, 25, 26, 27, 28, 29, or 30 nucleotide residues. In other embodiments, SPN magnetic particle conjugates may be linked to the surface of an electrode through the short linker.

Electrodes may include, but are not limited to metallised, non-metallised and mediator modified carbon graphite pastes, gold and platinum pastes. It may be Au/Ni/copper/gold low electrical resistance electrodes. Other electrodes include, but are not limited to gold electrodes, glassy carbon electrodes, an inert metal in an ionically conducting composite, and composite electrodes combining a polymeric material and electrically conducting particles. The electrode may be obtained by any manufacturing process known in the art, including the screen printing technique for making a SPE. Suitable composite potentiometric electrodes for selective analyte detection may be those disclosed in PCT application publication NO.: WO2005/103664; the contents of which are incorporated herein by reference in its entirety, and electrodes coated with an ionically conductive hydrophilic, preferably negatively charged, matrix such as gelatin type B or an equivalent (e.g., PCT application publication NO.: WO2015/001050; the contents of which are incorporated herein by reference in its entirety).

SPNs may be immobilized on the surface of the electrode using, e.g. coated with, ionically conductive hydrophilic matrices, preferably negatively charged hydrophilic protein matrices like gelatin B.

SPNs, aptamers and/or SPN magnetic particle conjugates of the present invention may be labeled with an electroactive reporter (e.g., a redox indicator). Electroactive reporters (signal transducers/redox indicators) may include, but are not limited to, 7-dimethyl-amino-1,2-benzophenoxazinium salt (Meldola's blue, MDB), methylene blue (MB), ferrocence, ferrocence-bearing polymers, ruthenium complexes, and Fe(CN)6^{4-/3-}.

### Target Analytes

Aptamer-magnetic particle detection agents, as discussed hereinabove, may bind any target analyte. As stated below, the target analyte may be an allergen protein or variants thereof. In some embodiments, aptamer-magnetic particle detection agents may be designed to bind or associate with proteins or other biomolecules which themselves associated with the allergen. In some embodiments, target analytes that can be detected using aptamer-magnetic particle agents may be allergens.

### Allergen

As used herein, the term “allergen” refers to a substance that can cause allergic reaction. An allergen is then a type of antigen that triggers an abnormally vigorous immune response in body.

Allergens include those from food products, the environment such as pollen, or animals such as a domestic pet dander. Food allergens include, but are not limited to proteins in legumes such as peanuts, peas, lentils and beans, tree nuts, wheat, milk, fish, egg white and sea food. Other allergens may be from the environment such as pollens, other animals (e.g., pet), pathogens and medicines. A comprehensive list of allergenic proteins from various sources is discussed below.

In some embodiments, allergens are food allergens. Examples of allergenic proteins associated with food include, but are not limited to, Brine shrimp (Art fr 5), Crab (Cha f 1), North Sea Shrimp (Cra c 1, Cra c 2, Cra c 4, Cra c 5, Cra c 6, Cra c 8), American lobster (Hom a 1, Hom a 3, Hom a 6), white shrimp (Lit v 1, Lit v 2, Lit v 3, Lit v4), giant freshwater prawn (Mac r 1), shrimp (Met e 1, Pen a 1, Pen i 1), northern shrimp (Pan b 1), spiny lobster (Pan s 1), black tiger shrimp (Pen m 1, Pen m 2, Pen m 3, Pen m 4, Pen m 6), narrow-clawed crayfish (Pon i 4, Pon i 7), blue swimmer crab (Por p 1), domestic cattle (Bos d 4, Bos d 5, Bos d 6, Bos d 7, Bos d 8, Bos d 9, Bos d 10, Bos d 11, Bos d 12), Atlantic herring (Clu h 1), common carp (Cyp c 1), Baltic cod (Gad c 1), Atlantic cod (Gad m 1, Gad m 2, Gad m 3), cod (Gad c 1), chicken (Gal d 1, Gal d 2, Gal d 3, Gal d 4, Gal d 5), Barramunda (Lat c 1), Lepidorhombus whiffiagonis (Lep w 1), chum salmon (Onc k 5), Atlantic salmon (Sal s 1, Sal s 2, Sal s 3) rainbow trout (Onc m 1), Mozambique tilapia (Ore m 4), edible frog (Ran e 1, Ran e 2), pacific pilchard (Sar sa 1), ocean perch (Seb m 1), yellowfin tuna (Thu a 1, Thu a 2, Thu a 3), swordfish (Xip g 1), abalone (Hal m 1), brown garden snail (Hel as 1), Squid (Tod p 1), pineapple (Ana c 1, Ana c 2), asparagus (Aspa o 1), barley (Hor v 12, Hor v 15, Hor v 16, Hor v 17, Hor v 20, Hor v 21), banana (Mus a 1, Mus a 2, Mus a 3, Mus a 4, Mus a 5), banana (Musxp1), rice (Ory s 12), rye (Sec c 20), wheat (Tri a 12, Tri a 14, Tri a 18, Tri a 19, Tri a 25, Tri a 26, Tri a 36, Tri a 37), maize (corn) (Zea m 14, Zea m 25), kiwi fruit (Act c1, Act c 2, Act c 5, Act c 8, Act c 10, Act d 1, Act d 2, Act d 3, Act d 4, Act d 5, Act d 6, Act d 7, Act d 8, Act d 9, Act d 10, Act d 11), cashew (Ana o 1, Ana o 2, Ana o 3), celery (Api g 1, Api g 2, Api g 3, Api g 4, Api g 5, Api g 6), peanut (Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13), brazil nut (Ber e 1, Ber e 2), oriental mustard (Bra j 1), rapeseed (Bran 1), cabbage (Bra o 3), turnip (Bra r 1, Bra r 2), bell pepper (Cap a 1w, Cap a 2), pecan (Car i 1, Car i 4), chestnut (Cas s 1, Cas s 5, Cas s 8, Cas s 9), lemon (Cit I 3), tangerine (Cit r 3), sweet orange (Cit s 1, Cit s 2, Cit s 3), Hazel (Cor a 1, Cor a 2, Cor a 8, Cor a 9, Cor a 11, Cor a 12, Cor a 13, Cor a 14), muskmelon (Cuc m 1, Cuc m 2, Cuc m 3), carrot (Dau c 1, Dau c 4, Dau c 5), common buckwheat (Fag e 2, Fag e 3), tartarian buckwheat (Fag t 2), strawberry (Fra a 1, Fra a 3, Fra a 4), soybean (Gly m 1, Gly m 2, Gly m 3, Gly m 4, Gly m 5, Gly m 6, Gly m 7, Gly m 8), sunflower (Hel a1, Hel a 2, Hel a 3), black walnut (Jug n 1, Jug n 2), English walnut (Jug r 1, Jug r 2, Jug r 3, Jug r 4), Cultivated lettuce (Lac s 1), Lentil (Len c 1, Len c 2, Len c 3), litchi (Lit c 1), narrow-leaved blue lupin (Lup an 1), apple (Mal d 1, Mal d 2, Mal d 3, Mal d 4), Cassava (Man e 5), mulberry (Morn 3), avocado (Pers a 1), green bean (Pha v 3), pistachio (Pis v 1, Pis v 2, Pis v 3, Pis v 4, Pis v 5), pea (Pis s 1, Pis s 2), apricot (Pru ar 1, Pru ar 3), sweet cherry (Pru av 1, Pru av 2, Pru av 3, Pru av 4), European plum (Pru d 3), almond (Pru du 3, Pru du 4, Pru du 5, Pru du 6), peach (Pru p 1, Pru p 2, Pru p 3, Pru p 4, Pru p 7), pomegranate (Pun g 1), pear (Pyr c 1, Pyr c 3, Pyr c 4, Pyr c 5), castor bean (Ric c 1), red raspberry (Rub i 1, Rub i 3), Sesame (Ses i 1, Ses i 2, Ses i 3, Ses i 4, Ses i 5, Ses i 6, Ses i 7), yellow mustard (Sin a 1, Sin a 2, Sin a 3, Sin a 4), tomato (Sola I 1, Sola I 2, Sola I 3, Sola I 4), potato (Sola t 1, Sola t 2, Sola t 3, Sola t 4), Mung bean (Vig r 1, Vig r 2, Vig r 3, Vig r 4, Vig r 5, Vig r 6), grape (Vit v 1), Chinese date (Ziz m 1), Anacardium occidentale (Ana o 1.0101, Ana o 1.0102), Apium graveolens (Api g 1.0101, Api g 1.0201), Daucus carota (Dau c1.0101, Dau c1.0102, Dau c1.0103, Dau c1.0104, Dau c1.0105, Dau c1.0201), Citrus sinensis (Cit s3.0101, Cit s3.0102), Glycine max (Gly m1.0101, Gly m1.0102, Gly m3.0101, Gly m3.0102), Lens culinaris (Len c1.0101, Len c1.0102, Len c1.0103), Pisum sativum (Pis s1.0101, Pis s1.0102), Lycopersicon sativum (Lyc e2.0101, Lyc e2.0102), Fragaria ananassa (Fra a3.0101, Fra a3.0102, Fra a3.0201, Fra a3.0202, Fra a3.0203, Fra a3.0204, Fra a3.0301), Malus domestica (Mal d1.0101, Mal d1.0102, Mal d1.0103, Mal d1.0104, Mal d1.0105, Mal d1.0106, Mal d1.0107, Mal d1.0108, Mal d1.0109, Mal d1.0201, Mal d1.0202, Mal d1.0203, Mal d1.0204, Mal d1.0205, Mal d1.0206, Mal d1.0207, Mal d1.0208, Mal d1.0301, Mal d1.0302, Mal d1.0303, Mal d1.0304, Mal d1.0401, Mal d1.0402, Mal d1.0403, Mal d3.0101w, Mal d3.0102w, Mal d3.0201w, Mal d3.0202w, Mal d3.0203w, Mal d4.0101, Mal d4.0102, Mal d4.0201, Mal d4.0202, Mal d4.0301, Mal d4.0302), Prunus avium (Pm av1.0101, Pru av1.0201, Pru av1.0202, Pru av1.0203), and Prunus persica (Pru p4.0101, Pru p4.0201); and any variants thereof. The names of allergens associated with food are systematically named and listed according to IUIS Allergen Nomenclature Sub-Committee (see, International Union of Immunological Societies Allergen Nomenclature Sub-Committee, List of isoallergens and variants.)

In addition to food allergens, signaling polynucleotides of the present invention may detect airborne particulates/allergens and other environmental allergens. Samples that contain allergens may be obtained from plants (e.g. weeds, grasses, trees, pollens), animals (e.g., allergens found in the dander, urine, saliva, blood or other bodily fluid of mammals such as cat, dog, cow, pig, sheep, horse, rabbit, rat, guinea pig, mouse and gerbil), fungi/mold, insects (e.g., stinging insects such as bee, wasp, and hornet and chirnomidae (non-biting midges), as well as other insects such as the housefly, fruit fly, sheep blow fly, screw worm fly, grain weevil, silkworm, honeybee, non-biting midge larvae, bee moth larvae, mealworm, cockroach and larvae of Tenibrio molitor beetle; spiders and mites such as the house dust mite), rubbers (e.g. latex), metals, chemicals (e.g. drugs, protein detergent additives) and autoallergens and human autoallergens (e.g. Hom s 1, Hom s 2, Hom s 3, Hom s 4, Hom s 5) (see, Allergen Nomenclature: International Union of Immunological Societies Allergen Nomenclature Sub-Committee, List of allergens and Allergen Nomenclature: International Union of Immunological Societies Allergen Nomenclature Sub-Committee, List of isoallergens and variants).

Examples of allergenic proteins from plants that can be detected using the compositions of the present invention include, but are not limited to, ash (Fra e 1), Japanese cypress (Cha o1, Cha o 2), sugi (Cry j1, Cry j 2), cypress (Cup a 1), common cypress (Cup s 1, Cup s 3), mountain cedar (Jun a 1, Jun a 2, Jun a 3, Jun s 1), prickly juniper (Jun o 4), eastern red cedar (Jun v 1, Jun v 3), sweet vernal grass (Ant o 1), saffron crocus (Cro s 1, Cro s 2), Bermuda grass (Cyn d 1, Cyn d 7, Cyn d 12, Cyn d 15, Cyn d 22w, Cyn d 23, Cyn d 24), orchard grass (Dac g 1, Dac g 2, Dac g 3, Dac g 4, Dac g 5), meadow fescue (Fes p 4), velvet grass (Hol I 1, Hol I 5), barley (Hor v 1, Hor v 5), rye grass (Lol p 1, Lol p 2, Lol p 3, Lol p 4, Lol p 11), bahia grass (Pas n 1), canary grass (Pha a 1, Pha a 5), timothy (Phl p 1, Phl p 2, Phl p 4, Phl p 5, Phl p 6, Phl p 7, Phl p 11, Phl p 12, Phl p 13), date palm (Pho d 2), Kentucky blue grass (Poa p 1, Poa p 5), rye (Sec c 1, Sec c 5, Sec c 38), Johnson grass (Sor h 1), wheat (Tri a 15, Tri a 21, Tri a 27, Tri a 28, Tri a 29, Tri a 30, Tri a 31, Tri a 32, Tri a 33, Tri a 34, Tri a 35, Tri a 39), maize (Zea m 1, Zea m 12), alder (Aln g 1, Aln g 4), redroot pigweed (Ama r 2), short ragweed (Amb a 1, Amb a 2, Amb a 3, Amb a 4, Amb a 5, Amb a 6, Amb a 7, Amb a 8, Amb a 9, Amb a 10, Amb a 11), western ragweed (Amb p 5), giant ragweed (Amb t 5), mugwort (Art v 1, Art v 2, Art v 3, Art v 4, Art v 5, Art v 6), sugar beet (Beta v 1, beta v 2), European white birch (Bet v 1, Bet v 2, Bet v 3, Bet v 4, Bet v 6, Bet v 7), turnip (Bra r 5), hornbeam (Car b 1), chestnut (Cas s 1), rosy periwinkle (Cat r 1), lamb's-quarters, pigweed (Che a 1, Che a 2, Che a 3), Arabian coffee (Cof a 1, Cof a 2, Cof a 3), Hazel (Cor a 6, Cor a 10), Hazel nut (Cor a1.04, Cor a2, Cor a8), European beech (Fag s 1), ash (Fra e 1), sunflower (Hel a 1, Hel a 2), para rubber tree (Hev b 1, Hev b 2, Hev b 3, Hey b 4, Hev b 5, Hev b 6, Hev b 7, Hev b 8, Hev b 9, Hev b 10, Hev b 11, Hev b 12, Hev b 13, Hev b 14), Japanese hop (Hum j 1), privet (Lig v 1), Mercurialis annua (Mer a 1), olive (Ole e 1, Ole e 2, Ole e 3, Ole e 4, Ole e 5, Ole e 6, Ole e 7, Ole e 8, Ole e 9, Ole e 10, Ole e 11), European hophornbeam (Ost c 1), Parietaria judaica (Par j 1, Par j 2, Par j 3, Par j 4), Parietaria officinalis (Par o 1), Plantago lanceolata (Pal I 1), London plane tree (Pla a 1, Pla a 2, Pla a 3), Platanus orientalis (Pla or 1, Pla or 2, Pla or 3), white oak (Que a 1), Russian thistle (Sal k 1, Sal k 2, Sal k 3, Sal k 4, Sal k 5), tomato (Sola I 5), Lilac (Syr v 1, Syr v 5), Russian-thistle (Sal k 1), English plantain (Pla 11), Ambrosia artemisiifolia (Amb a8.0101, Amb a8.0102, Amb a9.0101, Amb a9.0102), Plantago lanceolata (Pla11.0101, Pla11.0102, Pla11.0103), Parietaria judaica (Par j 3.0102), Cynodon dactylon (Cyn d1.0101, Cyn d1.0102, Cyn d1.0103, Cyn d1.0104, Cyn d1.0105, Cyn d1.0106, Cyn d1.0107, Cyn d1.0201, Cyn d1.0202, Cyn d1.0203, Cyn d1.0204), Holcus lanatus (Hol 11.0101, Hol 11.0102), Lolium perenne (Phl p1.0101, Phl p1.0102, Phl p4.0101, Phl p4.0201, Phl p5.0101, Phl p5.0102, Phl p5.0103, Phl p5.0104, Phl p5.0105, Phl p5.0106, Phl p5.0107, Phl p5.0108, Phl p5.0201, Phl p5.0202), Secale cereale (Sec c20.0101, Sec c20.0201), Betula Verrucosa (Bet v1.0101, Bet v1.0102, Bet v 1.0103, Bet v 1.0201, Bet v 1.0301, Bet v1.0401, Bet v 1.0402, Bet v 1.0501, Bet v 1.0601, Bet v 1.0602, Bet v1.0701, Bet v1.0801, Bet v1.0901, Bet v1.1001, Bet v1.1101, Bet v1.1201, Bet v 1.1301, Bet v1.1401, Bet v1.1402, Bet v1.1501, Bet v1.1502, Bet v1.1601, Bet v1.1701, Bet v 1.1801, Bet v1.1901, Bet v1.2001, Bet v1.2101, Bet v1.2201, Bet v1.2301, Bet v1.2401, Bet v 1.2501, Bet v1.2601, Bet v1.2701, Bet v1.2801, Bet v1.2901, Bet v1.3001, Bet v1.3101, Bet v 6.0101, Bet v6.0102), Carpinus betulus (Car b1.0101, Car b1.0102, Car b1.0103, Car b1.0104, Car b1.0105, Car b1.0106, Car b1.0106, Car b1.0106, Car b1.0106, Car b1.0107, Car b1.0107, Car b1.0108, Car b1.0201, Car b1.0301, Car b1.0302), Corylus avellana (Cor a1.0101, Cor a1.0102, Cor a1.0103, Cor a1.0104, Cor a1.0201, Cor a1.0301, Cor a1.0401, Cor a1.0402, Cor a1.0403, Cor a1.0404), Ligustrum vulgare (Syr v1.0101, Syr v1.0102, Syr v1.0103), Cryptomeria japonica (Cry j2.0101, Cry j2.0102), and Cupressus sempervirens (Cup s1.0101, Cup s1.0102, Cup s1.0103, Cup s1.0104, Cup s1.0105); and any variants thereof.

Lupin is an herbaceous plant of the leguminous family belonging to the genus Lupinus. In Europe, lupin flour and seeds are widely used in bread, cookies, pastry, pasta, sauces, as well as in beverages as a substitute for milk or soy, and in gluten-free foods. The International Union of Immunological Societies (IUIS) allergen nomenclature subcommittee recently designated □-conglutin as the Lup an 1 allergen. (Nadal, et al., (2012) PLoS one, 7(4): e35253), and more recently, a high-affinity 11-mer DNA aptamer against Lup an 1 (β-conglutin) was reported (Nadal, et al., (2013), Anal. Bioanal. Chem. 405:9343-9349).

Examples of allergenic proteins from mites that can be detected using the compositions of the present invention include, but are not limited to, mite (Blo t 1, Blo t 3, Blot 4, Blot 5, Blot 6, Blot 10, Blot 11, Blot 12, Blot 13, Blot 19, Blot t 21); American house dust mite (Der f 1, Der f 2, Der f 3, Der f 7, Der f 10, Der f 11, Der f 13, Der f 14, Der f 15, Der f 16, Der f 17, Der f 18, Der f 22, Der f 24); Dermatophagoides microceras (house dust mite) (Der m 1); European house dust mite (Der p 1, Der p 2, Der p 3, Der p 4, Der p 5, Der p 6, Der p 7, Der p 8, Der p 9, Der p 10, Der p 11, Der p 14, Der p 15, Der p 20, Der p 21, Der p 23); Euroglyphus maynei (House dust mite) (Eur m 2, Eur m 2, Eur m 3, Eur m 4, Eur m 14); storage mite (Aca s 13, Gly d 2, Lep d 2, Lep d 5, Lep d 7, Lep d 10, Lep d 13, Tyr p 2, Tyr p 3, Tyr p 10, Tyr p 13, Tyr p 24), Dermatophagoides farinae (Der f1.0101, Der f1.0102, Der f1.0103, Der f1.0104, Der f1.0105, Der f2.0101, Der f2.0102, Der f2.0103, Der f2.0104, Der f2.0105, Der f2.0106, Der f2.0107, Der f2.0108, Der f2.0109, Der f2.0110, Der f2.0111, Der f2.0112, Der f2.0113, Der f2.0114, Der f2.0115, Der f2.0116, Der f2.0117), Dermatophagoides pteronyssinus (Der p1.0101, Der p1.0102, Der p1.0103, Der p1.0104, Der p1.0105, Der p1.0106, Der p1.0107, Der p1.0108, Der p1.0109, Der p1.0110, Der p1.0111, Der p1.0112, Der p1.0113, Der p1.0114, Der p1.0115, Der p1.0116, Der p1.0117, Der p1.0118, Der p1.0119, Der p1.0120, Der p1.0121, Der p1.0122, Der p1.0123, Der p2.0101, Der p2.0102, Der p2.0103, Der p2.0104, Der p2.0105, Der p2.0106, Der p2.0107, Der p2.0108, Der p2.0109, Der p2.0110, Der p2.0111, Der p2.0112, Der p2.0113), Euroglyphus maynei (Eur m2.0101, Eur m2.0102), Lepidoglyphus destructor (Lep d2.0101, Lep d2.0101, Lep d2.0101, Lep d2.0102, Lep d2.0201, Lep d2.020) and Glycyphagus domesticus (Gly d2.0101, Gly d2.0201); and any variants thereof.

Examples of allergenic proteins from animals that can be detected using the compositions of the present invention include, but are not limited to, domestic cattle (Bos d 2, Bos d 3, Bos d 4, Bos d 5, Bos d 6, Bos d 7, Bos d 8), dog (Can f 1, Can f 2, Can f 3, Can f 4, Can f 5, Can f 6), domestic horse (Equ c 1, Equ c 2, Equ c 3, Equ c 4, Equ c 5), cat (Fel d 1, Fel d 2, Fel d 3, Fel d 4, Fel d 5w, Fel d 6w, Fel d 7, Fel d 8), mouse (Mus m 1), guinea pig (Cav p 1, Cav p 2, Cav p 3, Cav p 4, Cav p 6), rabbit (Ory c 1, Ory c 3, Ory c 4) rat (Rat n 1), Bos domesticus (Bos d 2.0101, Bos d 2.0102, Bos d 2.0103) and Equus caballus (Equ c2.0101, Equ c 2.0102); and any variants thereof

Examples of allergenic proteins from insects that can be detected using the compositions of the present invention include, but are not limited to, yellow fever mosquito (Aed a 1, Aed a 2, Aed a 3), Eastern hive bee (Api c 1), giant honeybee (Api d 1), honey bee (Api m 1, Api m 2, Api m 3, Api m 4, Api m 5, Api m 6, Api m 7, Api m 8, Api m 9, Api m 10, Api m 11, Api m 12), pigeon tick (Arg r 1), German cockroach (Bla g 1, Bla g 2, Bla g 3, Bla g 4, Bla g 5, Bla g 6, Bla g 7, Bla g 8, Bla g 11), bumble bee (Bom p 1, Bom p 4, Bom t 1, Bom t 4), silk moth (Bomb m 1), midge (Chi k 10, Chit 1, Chit 1.01, Chit 2, Chit 2. 0101, Chit 2. 0102, Chit 3, Chit 4, Chit 5, Chit 6, Chit 6. 01, Chit 7, Chit 8, Chi t 9), cat flea (Cte f 1, Cte f 2, Cte f 3), yellow hornet (Dol a 5), white face hornet (Dol m 1, Dol m 2, Dol m 5), biting midge (Fort 1, Fort 2), Savannah Tsetse fly (Glo m 5), Asian ladybeetle (Har a 1, Har a 2), silverfish (Lep s 1), booklouse (Lip b 1), Australian jumper ant (Myr p 1, Myr p 2, Myr p 3), American cockroach (Per a 1, Per a 3, Per a 6, Per a 7, Per a 9, Per a 10), Indian meal moth (Plo i 1, Plo i 2), wasp (Pol a 1, Pol a 2, Pol a 5, Pol e 1, Pol e 4, Pol e 5, Pol f 5, Pol g 1, Pol g 5, Pol m 5, Poly p 1, Poly s 5, Ves vi 5), Mediterranean paper wasp (Pol d 1, Pol d 4, Pol d 5), tropical fire ant (Sol g 2, Sol g 3, Sol g 4), Solenopsis invicta (red imported fire ant) (Sol I 1, Sol I 2, Sol I 3, Sol I 4), black fire ant (Sol r 2, Sol r 3), Brazilian fire ant (Sol s 2, Sol s 3), horsefly (Tab y 1, Tab y 2, Tab y 5), pine processionary moth (Tha p 1, Tha p 2), California kissing bug (Tria p 1), European hornet (Vesp c 1, Vesp c 5), Vespa magnifica (hornet) (Vesp ma 2, Vesp ma 5), Vespa mandarinia (Giant asian hornet) (Vesp m1, Vesp m 5), yellow jacket (Ves f 5, Ves g 5, Ves m 1, Ves m 2, Ves m 5), Vespula germanica (yellow jacket) (Ves p 5), Vespula squamosa (Yellow jacket) (Ves s 1, Ve s s5), Vespula vulgaris (Yellow jacket) (Ves v 1, Ves v 2, Ves v 3, Ves v 4, Ves v 5, Ves v 6), Blattella germanica (Bla g 1.0101, Bla g 1.0102, Bla g 1.0103, Bla g 1.02, Bla g 6.0101, Bla g 6.0201, Bla g 6.0301), Periplaneta Americana (Per a1.0101, Per a1.0102, Per a1.0103, Per a1.0104, Per a1.02, Per a3.01, Per a3.0201, Per a3.0202, Per a3.0203, Per a7.0101, Per a7.0102), Vespa crabo (Ves pc 5.0101, Ves pc 5.0101), Vespa mandarina (Vesp m 1.01, Vesp m 1.02); and any variants thereof.

Examples of allergenic proteins from fungi/mold that can be detected using the signaling polynucleotides and assays of the present invention include, but are not limited to, Alternaria alternata (Alternaria rot fungus) (Alt a 1, Alt a 3, Alt a 4, Alt a 5, Alt a 6, Alt a 7, Alt a 8, Alt a 10, Alt a 12, Alt a 13), Aspergillus flavus (fungus) (Asp f1 13), Aspergillus fumigatus (fungus) (Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 15, Asp f 16, Asp f 17, Asp f 18, Asp f 22, Asp f 23, Asp f 27, Asp f 28, Asp f 29, Asp f 34), Aspergillus niger (Asp n 14, Asp n 18, Asp n 25), Aspergillus oryzae (Asp o 13, Asp o 21), Aspergillus versicolor (Asp v 13), Candida albicans (Yeast) (Cand a 1, Cand a 3), Candida boidinii (Yeast) (Cand b 2), Cladosporium cladosporioides (Cla c 9, Cla c 14), Cladosporium herbarum (Cla h 2, Cla h 5, Cla h 6, Cla h 7, Cla h 8, Cla h 9, Cla h 10, Cla h 12), Curvularia lunata (Synonym: Cochliobolus lunatus) (Cur l 1, Cur I 2, Cur I 3, Cur I 4), Epicoccum purpurascens (Soil fungus) (Epi p 1), Fusarium culmorum (N.A.) (Fus c 1, Fus c 2), Fusarium proliferatum (Fus p 4), Penicillium brevicompactum (Pen b 13, Pen b 26), Penicillium chrysogenum (Pen ch 13, Pen ch 18, Pen ch 20, Pen ch 31, Pen ch 33, Pen ch 35), Penicillium citrinum (Pen c 3, Pen c 13, Pen c 19, Pen c 22, Pen c 24, Pen c 30, Pen c 32), Penicillium crustosum (Pen cr 26), Penicillium oxalicum (Pen o 18), Stachybotrys chartarum (Sta c 3), Trichophyton rubrum (Tri r 2, Tri r 4), Trichophyton tonsurans (Tri t 1, Tri t 4), Psilocybe cubensis (Psi c 1, Psi c 2), Shaggy cap (Cop c 1, Cop c 2, Cop c 3, Cop c 5, Cop c 7), Rhodotorula mucilaginosa (Rho m 1, Rho m 2), Malassezia furfur (Malaf2, Malaf3, Malaf4), Malassezia sympodialis (Malas1, Malas5, Malas6, Malas7, Malas8, Malas9, Malas10, Malas11, Malas12, Malas13) and Alternaria alternate (Alt a1.0101, Alt a1.0102); and any variants thereof.

Examples of additional allergens include, but are not limited to, Nematode (Ani s 1, Ani s 2, Ani s 3, Ani s 4), worm (Asc s 1), soft coral (Den n 1), rubber (Latex) (Hev b 1, Hev b 2, Hev b 3, Hev b 5, Hev b 6, Hev b 7, Hev b 8, Hev b 9, Hev b 10, Hev b 11, Hev b 12, Hev b 13), obeche (Trip s 1) and Heveabrasiliensis (Hev b6.01, Hev b6.0201, Hev b6.0202, Hev b6.03, Hev b8.0101, Hev b8.0102, Hev b8.0201, Hev b8.0202, Hev b8.0203, Hev b8.0204, Hev b10.0101, Hev b10.0102, Hev b10.0103, Hev b11.0101, Hev b11.0102); and any variants thereof.

In some embodiments, SPNs and compositions of the present invention may be used in a hospital for clinical food allergy or allergy test and to identify food/allergen(s) to which a patient is allergic. In addition, SPNs and compositions of the present invention may be used as a carry-on tester for people who have food/environmental allergy, for example at home to test commercial food, or at restaurant to check dishes they ordered. The food sample could be fresh food, frozen food, cooled food or processed food containing animal derived meat and/or vegetables.

### Other Target Molecules

In some embodiments, SPNs and compositions of the present invention may detect other target molecules, including but not limited to, pathogens from a pathogenic microorganism in a sample, such as bacteria, yeasts, fungi, spores, viruses or prions; disease proteins (e.g., biomarkers for diseases diagnosis and prognosis); pesticides and fertilizers remained in the environment; and toxins. In other embodiments, SPNs and compositions of the present invention may bind to non-protein targets such as minerals and small molecules (e.g., antibiotics), drugs and inorganic ion.

### Allergen Detection Methods and Assays

In accordance with the present invention, aptamers, signaling polynucleotides (SPNs), SPN-magnetic particle conjugates, detection agents and compositions of the present invention may be used to, in a broad concept, detect any molecules in a sample in a large variety of applications, such as food safety, diagnostic and prognostic tests in civilian and battlefield settings, environmental monitoring/control, and military use for detection of biological weapons. Various methods and assays may be used in combination with the aptamers, SPNs, SPN-magnetic particle conjugates, detection agents and compositions of the present invention; the choice may depend on the application field.

Particularly the present invention provides methods of determining the absence, presence and/or quantity of one or more target allergens in a sample using reagents comprising aptamer-magnetic particle conjugates. In some embodiments, the detection assays and methods can be used in a hospital for clinical food allergy or allergy test and to identify food/allergen(s) to which a patient is allergic. Such assays and methods may be used to monitor allergen contamination in food industry. Additionally, they may also be used at home or in a restaurant by a person who has allergy to test the allergen content before he/she consumes the food.

Assays and methods for detecting the allergen content in a sample is applicable to foods containing the allergens without any restriction. Examples of foods are eggs, milk, meat, fishes, crustacea and mollusks, cereals, legumes and nuts, fruits, vegetables, beer yeast, and gelatin; more particularly, egg white and egg yolk of the eggs, milk and cheese of the milk, pork, beef, chicken and mutton of the meat, mackerel, horse mackerel, sardine, tuna, salmon, codfish, flatfish and salmon caviar of the fishes, crab, shrimp, blue mussel, squid, octopus, lobster and abalone of the crustacea and mollusks, wheat, rice, buckwheat, rye, barley, oat, corn, millet, foxtail millet and barnyardgrass of the cereals, soybean, peanut, cacao, pea, kidney bean, hazelnut, Brazil nut, almond, coconut and walnut of the legumes and nuts, apple, banana, orange, peach, kiwi, strawberry, melon, avocado, grapefruit, mango, pear, sesame and mustard of the fruits, tomato, carrot, potato, spinach, onion, garlic, bamboo shoot, pumpkin, sweet potato, celery, parsley, yam and Matsutake mushroom of the vegetables, the foods containing them, and the ingredients thereof (e.g., ovoalbumin, ovomucoid, lysozyme, casein, beta-lactoglobulin, alpha-lactoalbumin, gluten, and alpha-amylase inhibitor).

The foods could be fresh foods, frozen foods, cooled foods or processed foods containing animal derived meat and/or vegetables. These foods may be processed by heating, freezing, drying, salting, fermentation, enzymatic processing, etc.

In some embodiments, one or more signaling polynucleotides (SPNs) may be used, depending on the nature of the food matrixes. Some food contains several allergenic proteins, e.g., at least eight peanut proteins, such as Ara h1 and Ara h2, can potentially cause an immunological response. In such case, more than one signaling polynucleotides (SPNs) against more than one allergenic protein may be used in a mixed cocktail for detecting the absence or presence of peanut. In other aspects, some food matrixes such as fish, shellfish and mollusks, contain only one major allergenic protein. One or more SPNs that specifically bind to this major allergen protein may be used for allergen detection.

In some embodiments, allergen detection assays and methods of the present invention can detect a lower concentration of allergen in a food sample. The sensitivity of nucleic acid aptamers makes it possible to detect the presence of an allergen as low as 0.0001 ppm. In some aspects, the concentration or mass of allergen that can be detected may range from 0.001 ppm to 5 ppm, or from 0.001 ppm to 0.1 ppm, or from 0.1 ppm to 3 ppm, or from 1 ppm to 5 ppm, or from 5 ppm to 10 ppm. In some aspects, the concentration or mass of allergen in a food sample that can be detected may be 0.001 ppm, 0.002 ppm, 0.003 ppm, 0.004 ppm, 0.005 ppm, 0.006 ppm, 0.007 ppm, 0.008 ppm, 0.009 ppm, 0.01 ppm, 0.02 ppm, 0.03 ppm, 0.04 ppm, 0.05 ppm, 0.06 ppm, 0.07 ppm, 0.08 ppm, 0.09 ppm, 0.1 ppm, 0.2 ppm, 0.3 ppm, 0.4 ppm, 0.5 ppm, 0.6 ppm, 0.7 ppm, 0.8 ppm, 0.9 ppm, 1.0 ppm, 1.5 ppm, 2 ppm, 2.5 ppm, 3 ppm, 3.5 ppm, 4 ppm, 4.5 ppm, 5 ppm or 10 ppm.

In general, methods for detecting the presence, absence and/or quantity of a target allergen in a sample comprise collecting and processing a food sample which is suspected to contain one or more target allergens of interest; contacting the food sample with SPN-magnetic particles in which the aptamer is a single-stranded nucleic acid having 20 to 200 nucleotides capable of specifically binding to a target allergen; detecting the allergen-SPN-magnetic particle complexes formed during the assay; and determining whether the target allergen is present in the food sample and the quantity of the target allergen in the sample as illustrated in FIG. 1B. The present assays and methods may have various forms depending on biosensors, detection kits and detection devices and systems used to implement the assays. SPN-magnetic particle conjugates may be used in any steps of the assays, such as capture agents to capture a target analyte (e.g., a target allergen or a specific epitope of the target allergen) in a sample; or detector agents for signaling detection; or competitive binding agents, or affinity agents to selected a target analyte (e.g., an allergen) bound magentic particles.

### Detection Assays Using Biosensors, Detection Systems and Devices by Ayub et al.

In some embodiments, methods of the present invention for detecting the absence, presence and/or quantity of a target allergen in a test sample comprise: (a). obtaining a test sample which is suspected to contain the target allergen; (b). placing the test sample into a sample analysis cartridge, wherein the cartridge comprises an input tunnel configured for receiving the test sample, a plurality of reservoirs which separately store sample preparation reagents and a substrate, and an analysis channel; (c). mixing the test sample with the sample preparation reagents stored in the reservoirs sequentially from the first reservoir, the second reservoir and the third reservoir, and so on, wherein the target allergen is hybridized with the preparation reagents; (d). initiating a testing protocol in a specialized computer which is configured to detect the sample analysis cartridge; (e). releasing the contents of the plurality of reservoirs into the analysis channel of the sample analysis cartridge wherein one or more sensors are disposed on the analysis channel to detect the hybridized target allergen; and (f). processing and analyzing the detection signals to identify the absence, presence, and/or the quantity of the allergen in the test sample. The detection devices and systems used to implement the present assay are described in detail by Ayub et al. in the PCT patent application publication No.: WO2014/164933; U.S. Pat. Nos. 9,207,245; 9,207,244; 9,086,417; 9,034,168; and 9,052,275; and U.S. patent application publication No.: US 2014/0336083; the contents of each of which are incorporated herein by reference in their entirety.

In some embodiments, the sample preparation reagents stored in the reservoirs within the sample analysis cartridge include a plurality of magnetic particles having surface-bound affinity molecules, a plurality of detector agents or a plurality of competitive binding agents, and a plurality of agents that facilitate the binding between the target allergen and the surface affinity molecules and the detector agents if the detector agents are used for the detection assay, or the surface affinity molecules and the competitive binding agents if the competitive binding agents are used for the detection assay. Accordingly, the plurality of magnetic particles within the reservoir may be conjugated to the present SPNs that specifically bind the target allergen which serve as surface bound affinity molecules. The target allergen in the test sample can bind the surface bound affinity molecules, i.e., SPNs specific to the target allergen. In some examples, the present SPNs may be used as detector agents if the detector agents are used for the detection assay, or competitive binding agents, each competitive binding agent including a pre-bound target allergen bound to a signaling agent, if the competitive binding agents are used for the detection assay.

In some embodiments, the present SPNs used for target allergen detection comprise the nucleic acid sequences of SEQ ID NOs.: 1-353 (See Tables 1 and 2). In some aspects, the SPNs with different sequences but having a high affinity and specificity to the same target allergen may be used in combination as affinity molecules, detector agents or competitive binding agents, to capture the same target allergen during the detection assay. In other aspects, the SPNs of the present invention may be used in combination with antibodies that bind the same target allergen in a detection assay. As a non-limiting example, the affinity molecule bound to the surface of magnetic particles may be a SPN specific to a target allergen, and an antibody specifically against the same target allergen may be used to construe the detector agent or competitive binding agent.

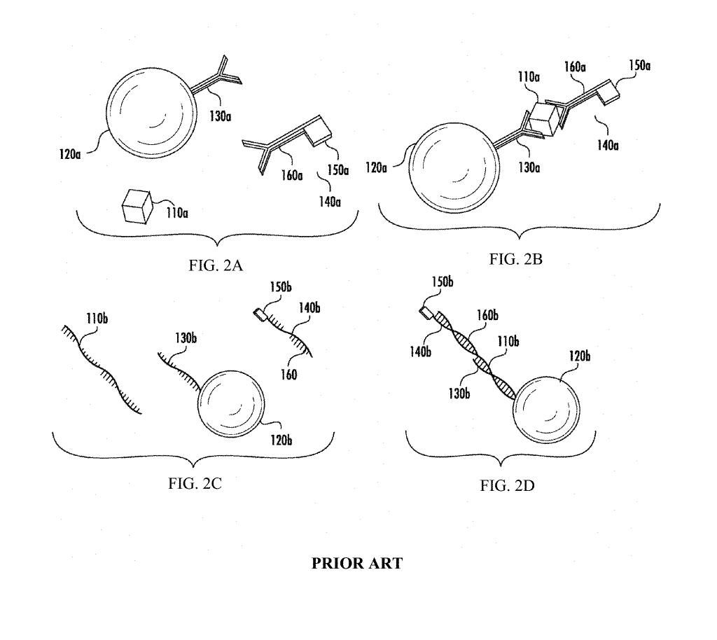

In some embodiments, a sandwich assay may be implemented. As shown in FIG. 2A and FIG. 2B, the present SPNs may be the affinity molecule 130a and 130b bound to the surface of magnetic particles 120a and 120b. Similarly, the present SPNs may replace the antibody 160a conjugated to a signaling agent 150a in the detector agent 140a, and the labeled nucleic acid probe 160b conjugated to a signaling agent 150b in the detector agent 140b. The signaling agent 150 may be a redox compound, a fluorescent marker (e.g., a fluorophore), an enzyme (e.g., horseradish peroxidase and soybean peroxidase) and any other detectable signaling agents.

In this context, sandwich complexes are formed when the various sample preparation reagents including magnetic particles 120, SPNs 130 and detector agents 140, hybridize together. As illustrated in FIG. 2C and FIG. 2D, each sandwich complex 100a and 100b is formed of a magnetic particle 120a, 120b having a surface bound SPN 130a, 130b; a target allergen 110a, 110b; and a detector agent 140a, 140b. The detailed description of the sample preparation reagents are described in the PCT patent application publication NO.: WO2014/164933; the content of which is incorporated by reference in its entirety.

In other embodiments, as shown in FIGS. 2A and 2B in the PCT patent application publication NO.: WO2014/164933, the present SPNs may be the affinity molecule 230 bound to magnetic particle 220, and the affinity molecule 260 bound to the signaling molecule 250. In this context, the sample preparation reagents in the sample analysis cartridge include a population of magnetic particles 220, each having a SPN as affinity molecule 230 bound to its surface; and a population of competitive binding agents 240, each including a pre-bound target allergen 270 which binds indirectly to the signaling agent 250 through a SPN (i.e., an affinity molecule) 260. During the process of the detection assay, the free unbound target allergen 210 present in s test sample and the competitive binding agent 240 compete with each other to bind to the surface affinity molecules 230 on the magnetic particles 220. The amount of competitive binding agent 240 and signaling agent 250 that successfully bind to the magnetic particle 220 is inversely proportional to the amount of unbound target allergen 210 present in the test sample.

The plurality of magnetic particles may include magnetic particles of two or more different sizes, each size having a different SPN coupled as surface-bound affinity molecule such that each size binds to a different target allergen. Similar to the signaling agent 150, the signaling agent 250 may be a redox compound, a fluorescent marker (e.g., a fluorophore), an enzyme (e.g., horseradish peroxidase and soybean peroxidase) and any other detectable signaling agents.

In some embodiments, sensors that can detect the complexes formed of the target allergen and SPN-magnetic particles may be disposed in a portion of the analysis channel within the sample analysis cartridge. The analyte reader device may include a magnet aligned with the sensors, a circuit and a processor as described in WO2014/164933 (the content of which is incorporated herein by reference in its entirety).

In some embodiments, the signaling agents 150 and 250 may be an oxidizing enzyme for example a peroxidase. The oxidizing enzyme bound to a target allergen and magnetic particle will induce, when its substrate is present in the sample analysis cartridge, an oxidation reaction and generate a detectable electrochemical signal, indicative of the absence, presence and/or quantity of the target allergen in the test sample.

In accordance with the present invention, the detection assays may be tailored for detection of one target allergen in a test sample, including one population of the sample preparation agents such as one population of magnetic particles at one size having particular SPNs bound to the surfaces, and one population of detector agents or competitive binding agents having the same SPNs. In other embodiments, the detection assays may comprise more than one populations of the sample preparation reagents including more than one magnetic particles having different SPNs bound to their surfaces, and more than one populations of detector agents and/or competitive binding agents, each population of the sample reagents is constructed to detect a different target allergen in the test sample.

In addition to SPNs and magnetic particles complexes, other reagents include agents that facilitate the formation of magnetic particle bound complexes, such as extraction buffers for lysis and extraction of target allergens from a test sample, salts that enhance the binding of the sample preparation reagents, detergents and blockers.

In some embodiments, the test sample may be collected using the sample collection devices in the PCT patent application publication No.: WO2014/164,933 to Ayub et al (the content of which is incorporated herein by reference in its entirety). A test sample may be delivered to the sample analysis cartridge by inserting the sample collection device loaded with the test sample into an input tunnel within the cartridge.

In some embodiments, the collected test sample may be transferred to the sample analysis cartridge through an input tunnel of the cartridge, wherein the reactions needed to detect the absence, presence and/or quantity of target allergens in a test sample occur. The sample received from the sample collection device may be mixed and hybridized with the sample preparation reagents stored in the reservoirs of the cartridge. The hybridized target allergens may be localized, for example by a magnetic force, over sensors embedded within the analysis channel of the cartridge for detection. A substrate may also be stored in a separate reservoir of the cartridge, when the signaling agent is an oxidizing enzyme. The substrate is transferred to the analysis channel of the cartridge and introduced to the hybridized target allergens to undergo a detectable reaction. An electrochemical signal is generated. Various embodiments of the sample analysis cartridge are described in detail in the PCT patent application publication No.: WO2014/164,933 to Ayub et al; the content of which is incorporated herein by reference in its entirety.

In some embodiments, the analysis channel within the cartridge includes a circuit board component on which one or more sensors are disposed. In some aspects, the sensors are formed of gold or other conducting metals if the signaling agent is an oxidizing enzyme. Such sensors will detect target allergens when an enzyme substrate is provided to induce the electrochemical reaction with the hybridized target allergens within the analysis channel. Signals detected by the sensors may be delivered to a reader device for processing. The more detailed description of the sensors are included in the PCT patent application publication No.: WO2014/164,933 to Ayub et al; the content of which is incorporated herein by reference in its entirety.

In some embodiments, the detection signals are processed and displayed by a reader device. The reader device may be a computer, an iPad and/or a cellphone, or other processors that can execute one or more methods for detecting the absence, presence and/or quantity of the target allergens. As a non-limiting example, the reader device by Ayub et al (WO2014/164,933; the content of which is incorporated herein by reference in its entirety) may be used to detect the absence, presence and/or quantity of target allergens as described in the present disclosure.

### Detection Assays Using Filtration Devices and On-Chip Biosensors by Florescu et al.

In other embodiments, methods of the present invention for detecting the absence, presence and/or quantity of a target allergen in a test sample comprise: (a). obtaining a test sample which is suspected to contain the target allergen; (b). filtering the test sample using a filter configured to filter the test sample resulting in a filtrate comprising the target allergen; (c). delivering the filtrate of step (b) through a capillary to the surface of an integrated circuit which includes one or more sensor areas on the surface of said integrated circuit, wherein dried magnetic particles whose surfaces are functionalized to react with the target allergen in the filtrate are pre-stored in the capillary channel, and wherein the filtrate flows in the capillary channel and the target allergen in the filtrate binds the functionalized magnetic particles to form allergen magnetic particle complexes which can bind specifically onto the sensor areas on the surface of the integrated circuit; (d). detecting magnetic particles specifically bound to said one or more sensor areas using a plurality of sensors; and (e). transmitting the signals detected in step (d) into indicative of the absence, presence and/or quantity of the target allergen in the test sample. The present assays may be implemented using the filtration device and on-chip biosensors disclosed by Florescu et al., in U.S. Pat. Nos. 8,614,572; 8,895,320; 9,244,068; U.S. patent application publication No.: US2013/0230913; and PCT patent application publication No.: WO2014/189624; the contents of each of which are incorporated herein by reference in their entirety.

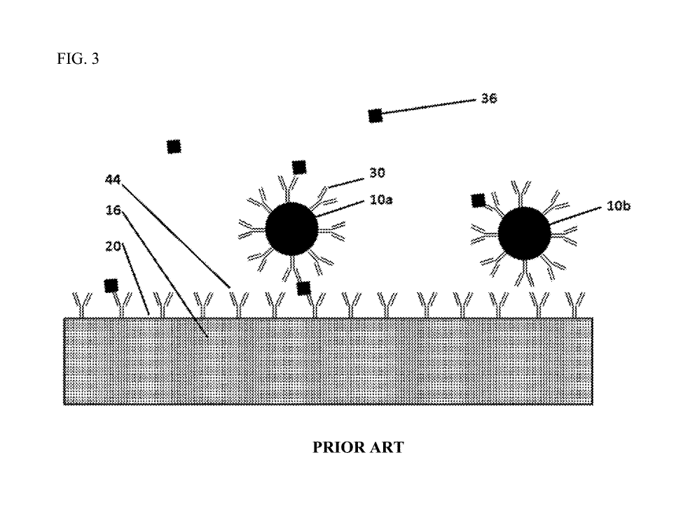

In some embodiments, the present SPNs may be used to functionalize the surfaces of magnetic particles included in the capillary channel, the sensor areas on the surface of the integrated circuit, or the combination thereof, of the filtration device as disclosed in U.S. Pat. Nos. 8,895,320 and 9,244,068 and biosensors as disclosed in U.S. patent application publication NO.: 2013/0230913 and PCT patent application publication NO. WO2014/189624; the contents of each of which are incorporated herein by reference in their entirety. In one example, the dried magnetic particles in the capillary channel of the filtration device (U.S. Pat. Nos. 8,895,320 and 9,244,068) may be functionalized by conjugating the present SPNs to the surfaces of the magnetic particles. The SPNs functionalized magnetic particles may capture the target allergen in the filtrate by their specific affinity to the target allergen. Allergen bound magnetic particles, when flow to the surface of the integrated circuit, will bind the sensor areas on the surface of the integrated circuit and be manipulated by the magnetic field created by the magnetic field generators of the filtration device by Florescu (U.S. Pat. Nos. 8,895,320 and 9,244,068). As shown in FIG. 3, the capture antibody 30 which are conjugated to magnetic particles 10b, may be replaced by the SPNs of the present invention. Similarly, the surface antibody 44 on the surface 20 of the integrated circuit 16 may be replaced by the SPNs of the present invention.

In addition to functionalize the magnetic particles to capture target allergen in the test sample, SPNs of the present invention may also be used to coat the sensor areas on the surface of the integrated circuit (FIG. 3). The SPN coated sensor areas will bind specifically to allergen bound magnetic particles and trap such magnetic particle complexes. Free magnetic particles that are not bound to target allergens may be separated from the sensor areas by different magnetic concentration field. The magnetic particles bound to the sensor areas may be detected by a plurality of sensors.

In some embodiments, the present SPNs used for target allergen detection comprise the nucleic acid sequences of SEQ ID NOs.: 1-353 (See Tables 1 and 2). In some aspects, the SPNs with different sequences but having a high affinity and specificity to the same target allergen may be used in combination. In other aspects, the SPNs may be used in combination with antibodies against the same target allergen.

In some embodiments, the capillary channel configured to deliver the filtrate to the surface of the integrated circuit (IC). The IC may be placed with one or more sensor areas directly below the outlet of the capillary channel. The SPN functionalized magnetic particles and other reagents may be stored in a dried state in the filter, the capillary channel and/or the sensor areas on the surface of the integrated circuit. The target allergen in the test sample filtrate, when flows from the filter membrane, the capillary channel to the sensor areas on the surface of the integrated circuit, may bind the SPN functionalized magnetic particles. Magnetic particle bound to the target allergen can bind specifically through specific chemical interactions to the chemically functionalized sensor areas on the surface of the IC.

In accordance with the present invention, detection assays mediated by the present SPNs further comprise a step of manipulating the magnetic particles bound to said one or more sensor areas on the surface of the integrated circuit before detecting magnetic particles specifically bound to said one or more sensor areas, wherein the magnetic particles are manipulated by one or more magnetic field generators. In some aspects, the magnetic field generators may be one or more magnetic concentration field generators which generate one or more concentration fields to pull the specifically bound magnetic particles to one or more sensor areas on the surface of the integrated circuit. In other aspects, the magnetic field generator may be one or more magnetic separation field generators which generate one or more separation fields to remove the non-specifically bound magnetic particles from one or more sensor areas on the surface of the integrated circuit. As a non-limiting example, the magnetic field generation platform disclosed by Florescu et al in U.S. Pat. No. 8,614,572 (the content of which is herein incorporated by reference in its entirety) may be integrated to manipulate magnetic particles in the present detection assays.

In some embodiments, the filter may be a porous filter membrane or a filter assembly. As non-limiting examples, the filter may be those disclosed in U.S. Pat. Nos. 8,895,320; 9,244,068; U.S. patent application publication No.: US2013/0230913; and PCT patent application publication No.: WO2014/189624; the contents of each of which are herein incorporated by reference in their entirety.

In accordance to some embodiments of the present invention, a plurality of magnetic particle sensors are embedded in the integrated circuit; the magnetic particle sensors are capable of detecting magnetic particles specifically bound to the one or more sensor areas on the surface of the integrated circuit (U.S. Pat. Nos. 8,895,320; and 9,244,068; the contents of each of which are incorporated herein by reference in their entirety). In some aspects, magnetic particle sensors may be placed outside of the sensor areas of the integrated circuit to count non-specifically bound magnetic particles, which is excluded from detection signal. The number of magnetic particles specifically bound to the sensor areas on the surface of the integrated circuit is representative of the concentration of the target allergen in the test sample.

In other embodiments, other types of sensors may be embedded in the sensor areas on the surface of the integrated circuit to detect magnetic particle bound to the target allergen. In some examples, the sensors are a plurality of optical sensors formed on the surface of the integrated circuit. Target allergen-magnetic particle complexes specifically bound to the sensor areas can cast optical shadows that reduce the amount of light transmitted from the light source to the optical sensors. The shadow refers to any type of light modulation such as intensity, spectrum and polarization. The amount of light detected by the optical sensors is representative of the presence and number of the magnetic particles bound to the sensor areas on the surface of the integrated circuit. A detailed description of optical sensors is disclosed by Florescu in U.S. patent application publication NO.: US2013/0230913, the content of which is herein incorporated by reference in its entirety. In other examples, magnetic particles with different colors may be used to capture the target allergen and optical sensors may be used to detect magnetic particles of different colors (See PCT patent application publication NO.: WO2014/189624; the content of which is herein incorporated by reference in its entirety).

Detection Assays Using Electrical and/or Electrochemical Detection

In some embodiments, methods of the present invention for detecting the absence, presence and/or quantity of a target allergen in a test sample comprise: (a). obtaining a test sample which is suspected to contain the target allergen; (b). obtaining a processed extract from the test sample of step (a) having the target allergen. (c). contacting the processed extract with detection agents specific to the target allergen, wherein the detect agents are labeled with an electroactive reporter; (d). detecting electrical and/or electrochemical signal generated upon the binding of the target allergen to the detect agents; and (e). transmitting the signals detected in step (d) into indicative of the absence, presence and/or quantity of the target allergen in the test sample.

In accordance with the present invention, detection agents and/or other reagents/biomolecules are coated to the electrode surface, for example a gold electrode surface. Conducting polymers such as AuNRs, AuNPs modified conducting polymers can be used as materials for immobilization of detect agents on the surface of the electrode.

Electronic biosensors with diverse signaling approaches may be incorporated for signal detection. In some embodiments, the electroactive reporter (e.g., a redox indicator) may be covalently linked to one end of the SPN. The free SPNs or the SPN magnetic particle conjugates are coated to the surface of an electrode.

In some aspects, it may be a “signal off” biosensor. SPN can be attached to two signaling moieties and the signal produced by the sensor is dependent on the structural changes in the SPN following target analyte binding. As a non-limiting example, a SPN may be attached to a gold electrode via its 5′ end and linked to methylene blue via its 3′ end, or vice versa. In the absence of the target analyte, the SPN adopts a flexible, folded configuration, bringing methylene blue close to the surface of the electrode and promoting electron transfer from methylene blue to the electrode when the two components collide. In the presence of the target analyte, the SPN is driven to fold into its binding competent guanine-quartet conformation, which in turn moves the methylene blue away from the electrode surface and prohibits electron transfer due to its rigidity.

In some aspects, it may be a “signal-on” biosensor. In this system, the target analyte binding induces folding of the SPN into a three-way junction structure, thus decreasing the resistance of electron transfer. The conformation change due to the target analyte binding brings electroactive reporter/signal transducer closer to the electrode, thereby favoring the collision and electron transfer between them.

In other aspects, it may be a competition based assay for detection. SPN labeled with an electroactive reporter (e.g., MB) may not be directly attached to the surface of the electrode. Rather, the electrode is functionalized with a nucleic acid sequence that is complementary to the SPN or aptamer sequence. When the SPN or aptamer is not bound by the target analyte, it hybridizes with the complementary DNA (cDNA), so that the electroactive reporter (e.g., MB) is brought to the proximity of the electrode. When the SPN or aptamer binds to the target analyte, the hybridization is disrupted and this duplex-to-complex structural change is reported by a decrease in the electrochemical signal.

In some embodiments, the electroactive reporter may be added in trans to the SPN-linked electrode in order to produce a signal. As a non-limiting example, the SPN is attached to the surface of a gold electrode through its 5′ terminus. An electroactive reporter linked to a short oligonucleotide complementary to the 3′ terminus of the SPN may be added to the reaction mixture. In the absence of the target analyte, the reporter is brought to interact with the electrode and generate an electrical/electrochemical signal. In the presence of the target analyte, the target competes with the complementary oligonucleotide and the electrical/electrochemical signal is decreased because of the competition.

In other embodiments, two complementary oligonucleotides may be bound to a surface connected to a positively charged electrode and a negatively charged electrode, respectively. The electroactive reporter labeled SPN or SPN magnetic conjugate binds to both complementary sequences and generates an electrical/electrochemical signal. In the presence of the target analyte, the binding of the target analyte to the SPN interrupts the connection of the two electrodes and decreases the electrical/electrochemical signal.

The complementary oligonucleotide may contain 5 to 30 nucleotide residues, for instance, 5, 6, 7, 8, 9, 10, 11, 12, 13, 14, 15, 16, 17, 18, 19, 20, 21, 22, 23, 24, 25, 26, 27, 28, 29 or 30 nucleotide residues.

Electrochemical sensors may be fabricated using several techniques including, but not limited to, Electrochemical Impedance Spectroscopy (EIS), potentitometry with Electrochemiluminescence or electrogenerated chemiluminescence (ECL), Cyclic voltammetry (CV) and Differential Pulse Voltammetry (DPV).

### Detection Assays Using MEMS Devices

In some embodiments, MEMS devices may be used in combination with the present aptamers, signal polynucleotides (SPNs), aptamer/SPN magnetic particle conjugates for detecting the absence, presence and/or quantity of a target allergen in a test sample.

MEMS (Micro-electromechanical systems) is a technology that combines computers with tiny mechanical devices such as sensors, embedded in semiconductor chips. While the functional elements of MEMS are miniaturized structures, sensors, actuators and microelectrics, the most notable elements are the microsensors and microactuators, which together can be appropriately categorized as “transducers”. The transducers are defined as converting energy from one form to another. The microsensors embedded in MEMS devices typically convert a measured mechanical signal into an electrical signal. The microsensors can sense a variety of modalities including temperature, pressure, inertial forces and other mechanical phenomena, chemical species, magnetic fields, radiation, optical changes, biological reactions, etc. In accordance with the present invention, Aptamers/SPNs and aptamer/SPN-based biosensors may be integrated to MEMS devices for detection of allergen in a sample. Generally, aptamers/SPNs and aptamer/SPN-based microbiosensors, and other key elements (such as actuators, and structures) can all be merged onto a common silicon substrate (e.g., a single microchip) along with integrated circuits (IC) (i.e., microelectronics). The mechanical and/or electromechanical signals are transformed to electrical signals for detection. The electronics then process the information derived from the sensors.

In some embodiments, MEMS biosensors include microcantilever based sensors. This approach involves the use of microcantilevers as signal transducers. The SPN detects and binds a target analyte. The change in binding state of the SPN is subsequently communicated either directly or through a “bridge” element to the cantilevers, prompting this signal transducer to produce a signal to report the interaction between the SPN and the target analyte. In some examples, two cantilevers are used as the signaling transducers; one is attached the detection agent (e.g., aptamer/SPN magnetic particle conjugates) which serves as the sensor; the other has a control aptamer (e.g., a non-specific DNA/RNA sequence) which serves as the reference. In the presence of the target analyte, the SPN binds to the target and generates forces which act on the surface of cantilevers. The mass loaded cantilevers will bend and deflect. Thus, target recognition may be evaluated by measuring the extent of cantilever deflection or changes in resonance frequency, force constant, or other capacities by the integrated MEMS device. The mechanical changes caused by the interaction between two sequences upon the target analyte binding are calculated and the non-specific binding and other background disturbances may be eliminated by subtracting the deflection of the reference cantilever from the deflection of the sensor cantilever.

In some aspects, the cantilever may be coated with gold in order to permit the covalent linkage of sequences.

In accordance with the present detection assays, MEMS devices may be fabricated using any techniques known in the art, for example, those disclosed in U.S. Pat. No. 7,785,912 to Hartzell J W et al.; U.S. Pat. No. 7,875,483 to Izumi K et al.; U.S. Pat. No. 8,174,342 to Ebin L et al.; U.S. Pat. No. 8,278,919 to Edelstein A et al.; U.S. Pat. No. 8,384,169 to Langebrake L et al.; U.S. Pat. No. 8,451,078 to Chiu C et al.; U.S. Pat. No. 9,000,656 to Peterson K E et al.; U.S. Pat. No. 9,048,052 to Birkholz M et al.; and U.S. Pat. No. 9,162,877 to Chang J B et al.; and U.S. application publication NOs.: US2014/0206074 to Peterson K E et al.; US2015/0203345 to Ramchandra P A et al.; the contents of each of which are incorporated herein by references in their entirety.

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Activity: As used herein, the term “activity” refers to the condition in which things are happening or being done. Compositions of the invention may have activity and this activity may involve the binding to a target molecule.

Allergen: as used herein, the term “allergen” means a compound, substance or composition that causes, elicits or triggers and immune reaction in a subject. As such, allergens are typically referred to as antigens. An allergen is typically a protein or a polypeptide.

Allergen detection agent: As used herein, the term “an allergen detection agent” refers to Any agent which is capable of, or does, interact with and/or bind to one or more allergens in a way that allows detection of such allergen in a sample is referred to herein as an “allergen detection agent” or “detection agent”.

Analyte: As used herein, an “analyte” is a target of interest that can specifically interact with (bind to) an aptamer and be detected and/or measured. In the context of the present invention, an analyte may be an allergen.

Aptamer: as used herein, the term “aptamer” refers to single stranded nucleic acid. In general, aptamers refer to either an oligonucleotide of a single defined sequence or a mixture of said oligonucleotides, wherein the mixture retains the properties of binding specifically to a target allergen. A RNA aptamer is an aptamer comprising ribonucleoside units. RNA aptamer also meant to encompass RNA analogs as defined herein. A DNA aptamer an aptamer comprising deoxy-ribonucleoside units. DNA aptamer also meant to encompass DNA analogs as defined herein.

Binding affinity: as used herein, the term “binding affinity” is intended to refer to the tendency of an aptamer to bind or not bind a target and describes the measure of the strength of the binding or affinity of the aptamer to bind the target.

Detection: As used herein, the term “detection” means an extraction of a particular target protein from a mixture of many non-target proteins, indicating the absence, presence, and/or amount of a target protein from a mixture of many non-target proteins.

Electrochemical biosensor: The term “electrochemical biosensor”, as used herein, means an analytical device that consists of a sensitive biological recognition material targeting an analyte of interest and a transduction element for converting the recognition process into an amperometric or potentiometric signal. In the context of the present invention, the sensitive biological recognition material may be aptamer-derived signal polynucleotides (SPNs). One example of the analyte of interest is food allergen.

Magnetic particles: As used herein, the term “magnetic particles” refer to ( ). Magnetic particles may include magnetic microbeads and/or nanoparticles.

Oligonucleotide: as used herein, the term “oligonucleotide” is generic to polydeoxyribonucleotides (containing 2′-deoxy-D-ribose or modified forms thereof), i.e. DNA, to polyribonucleotides (containing D ribose or modified forms thereof), i.e. RNA, and to any other type of polynucleotide which is an N-glycoside or C-glycoside of a purine or pyrimidine base, or modified purine or pyrimidine base or abasic nucleotides. In the context of the present invention, the “oligonucleotide” includes not only those with conventional bases, sugar residues and internucleotide linkages, but also those that contain modifications of any or all of these three moieties.

As used herein, the terms “nucleic acid” “polynucleotide” and “oligonucleotide” are used interchangeable herein and refer to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Sample: As used herein, the term “sample” refers to any composition that might contain a target of interest to be analyzed including, but not limited to, biological samples obtained from subjects (including humans and animals as detailed below), samples obtained from the environment for example soil samples, water samples, agriculture samples (including plant and crop samples), or food samples. Food samples may be obtained from fresh food, processed/cooked food or frozen food.

Sensitivity: As used herein, the term “sensitivity” means the ability of a detection molecule to bind to a target molecule.

Specifically binds: as used herein, the term “specifically binds” means that an aptamer reacts or associates more frequently, more rapidly, with greater duration and with greater affinity with a particular target molecule, than it does with alternative target molecules. For example, an aptamer that specifically binds to a target allergen binds that allergen or a structural part or fragment thereof with greater affinity, avidity, more readily, and/or with greater duration than it binds to unrelated allergen protein and/or parts or fragments thereof. It is also understood by reading this definition that, for example, an aptamer that specifically binds to a first target may or may not specifically bind to a second target. As such, “specific binding” does not necessarily require exclusive binding or non-detectable binding of another molecule, this is encompassed by the term “selective binding”. The specificity of binding is defined in terms of the comparative dissociation constants (Kd) of the aptamer for target as compared to the dissociation constant with respect to the aptamer and other materials in the environment or unrelated molecules in general. Typically, the Kd for the aptamer with respect to the target will be 2-fold, 5-fold, or 10-fold less than the Kd with respect to the target and the unrelated molecule or accompanying molecule in the environment. Even more preferably, the Kd will be 50-fold, 100-fold or 200-fold less.

Target: as used herein, the term “target” and “target molecule” refers to a molecule which may be found in a tested sample and which is capable of binding to a detection molecule such as an aptamer or an antibody.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### Examples

### Example 1. Selection of Aptamers with Nucleic Acid Sequences that Bind an Allergen

An in vitro screening experiment based on SELEX method was carried out and aptamers were selected against the allergen targets including egg, gluten, milk, soy, fish, peanut, cashew and crustacean, over the counter-target (combinations of the non-target proteins) and were further engineered for their capability in detecting targeted food allergens.

### Experimental Plan

Various RNA libraries were used to select for binding ability in selection buffer consisting of 100 mM Tris (pH 8), 5 mM EDTA, 150 mM NaCl, 10 mM MgCl2, 0.1% SDS, 0.1% Gelatin, 1% NP-40 (Tergitol), 0.5% Deoxycholate Sodium at 23° C. A given round of selection began with incubating RNA library members in either the buffer alone (negative selection), then collecting the portion of the library that did not respond (i.e. cleave). The second part of each round (when called for) consisted of incubating the non-responsive molecules from the prior negative selection step with the full combination of non-positive targets (as the counter), or with just the selection buffer again for a second negative selection. Once again, the non-responsive (non-cleaving) molecules would be collected. The final step of each round consists of incubating the material from the previous step with the positive target (each of the allergens as appropriate) in buffer, then collecting the responsive material (i.e. cleaved RNA). Each selection round was followed by reverse transcription to generate cDNA, library amplification through PCR, and regeneration of the RNA library by transcription. After subjecting the initial library of diverse random sequences to varying consecutive rounds of selection (i.e. negative, counter and positive selections), again project-dependent, and the enriched libraries were divided into three fractions to perform the parallel assessments.

The parallel assessment of libraries enriched after rounds of negative, counter and positive selections, involves simultaneously exposing one third of the enriched library to selection buffer alone, another one-third to the counter-target complex in selection buffer, and the final one-third of the enriched library to the target allergen in buffer. Any residual RNA molecules that react indiscriminately to both target allergen and counter-targets, or that still generate a response in the absence of the target allergen were identified and discarded during further bioinformatics analysis.

The enriched RNA libraries after the parallel assessment were subjected to PAGE gel assessment. 40 pmoles of enriched library was exposed separately to the negative (buffer only), counter target, or target allergen (e.g., milk, wheat, egg white and peanut) in selection buffer. After 5 minutes incubation at 23° C., libraries exhibiting a positive response (i.e. cleavage) material were collected, ethanol precipitated, reverse transcribed, and PCR-amplified for sequencing and bioinformatics analysis.

### Materials and Methods

Targets (complexes of proteins from cashew, peanut, fish, milk, soy, gluten, egg and crustacean) were dried down, if necessary, before being combined with RNase-free water for preliminary analysis and aptamer screening. When needed, targets were pooled to produce counter-target mixture by combining appropriate amounts of the targets which were not designated as positive target for the selection. The initial aptamer library template and primers were synthesized by IDT (Coralville, Iowa) as single-stranded DNA. The library was then primer extended to provide double-stranded DNA (dsDNA) using Titanium Taq DNA polymerase from Clontech (Mountain View, Calif.).

Following the experimental plan, for a given generation of the library, RNA was transcribed from the previous dsDNA with AmpliScribe T7 Transcription kits from Epicentre (Madison, Wis.) and purified using a 10% denaturing polyacrylamide gel electrophoresis (PAGE). The purified RNA was combined with Selection Buffer, which was then diluted to 1× concentration (100 mM Tris (pH 8), 5 mM EDTA, 150 mM NaCl, 10 mM MgCl2, 0.1% SDS, 0.1% Gelatin, 1% NP-40 (Tergitol), 0.5% Deoxycholate Sodium) for negative selection. Negative selection began with a refolding cycle, which involved heating the sample to 65° C. to denature the RNA before bringing the sample to 23° C. for the remainder of the incubation. After incubation, non-cleaved RNA was separated from cleaved RNA using 10% denaturing PAGE. Recovered non-cleaved material was combined with counter-target and buffer, target and buffer, or buffer alone depending on the selection step, incubated at 23° C., and partitioned on 10% denaturing PAGE. Recovery and another selection step was implemented if called for. cDNA was then generated from eluted post-selection library using SuperScript II Reverse Transcriptase (Life Technologies; Carlsbad, Calif.), then PCR-amplified with Titanium Taq DNA polymerase (Clontech; Mountain View, Calif.) to complete the round of selection. After several rounds of selection steps, libraries were enriched and showed that the negative cleavage amount was less than 30%, and that there was at least 5% more cleavage in the positive treatment when compared to the counter.

The initial libraries consisting of approximately 10¹⁴ random sequences was subjected to varying rounds of ribozyme-based SELEX to enrich for sequences that bind to the target allergens and to eliminated sequences that bind to the counter-targets over multiple rounds of selection. As a result, the population to be sequenced is expected to contain multiple copies of potential aptamer candidates (Van Simaeys et al., Study of the Molecular Recognition of Aptamers Selected through Ovarian Cancer Cell-SELEX, 2010, PLOS One, 5(11): e13770).

### Sequencing and Bioinformatics

The Illumina (San Diego, Calif.) MiSeq system was implemented to sequence the aptamers after the selections using a paired-end read technique. Bioinformatics analysis of the sequencing data identified candidate aptamer molecules. The deep sequencing and subsequent data analysis reduced the traditional approach of performing a large number of selections, which may introduce error and bias due to the screening process (Schütze et al., Probing the SELEX Process with Next-Generation Sequencing, PLos One, 2011, 6(12): e29604).

### Selection of Aptamer Candidates

Sequence family construction focused on motif presence which means that a sequence's frequency in the positive target population was factored in, but places greater emphasis on the prevalence of sub-sequences in the overall population (100% match over the entire sequence not necessary to join a family). Two other factors were used to adjust the importance of motif-family size to determine candidate sequences. One factor is the presence of the sequence in the negative and counter-target population. Three libraries were collected from the parallel assessment: the positive target-exposed library, the buffer-only negative library, and the counter-target-exposed library. All libraries were analyzed to discover any sequences that have yet to be removed during a negative- or counter-selection step, but still have affinity for both the target and counter-target. A given sequence appears more frequently in the positive population than in the counter-target-exposed population, making it an attractive candidate for further testing.

The secondary structure of a given candidate sequence was also predicted using the Mfold secondary structure modeling software (Zucker, Mfold web server for nucleic acid folding and hybridization prediction, Nucleic Acids Res., 2003, 31 (13): 3406-3415).

A set of aptamer sequences were selected and further designed as signaling polynucleotides for detecting different food allergens, including cashew, peanut, egg white, wheat, fish, soy, milk and crustacean. The full sequences and core sequences which define the binding specificity to each allergen of selected aptamers are listed in Table 1. The selected aptamers for each food allergen are then further modified at either one or both of the 5′ terminus and the 3′ terminus to optimize the binding affinity to its targeted allergen. Modified sequences that are intended to have a fluorophore probe (e.g., Texas Red) at the 5′ terminus are the signaling polynucleotides that will be tested for allergen detection as described herein.

### Example 2. Generation of SPN and Magnetic Particle Conjugates

Aptamer-magnetic particle conjugates are generated using biotin-streptavidin system. Magnetic particles of different sizes are purchased from any commercial vendors. Aptamers that specifically bind peanut allergen are biotinalyted following standard procedure.

An aliquot of streptavidin-coated magnetic beads are washed first several times with the wash buffer (e.g., 1 M NaCl, 20 mM Tris, 1 mM EDTA, pH=7.5), and then resuspended in aptamer binding buffer (e.g., 1 M NaCl, 5 mM Tris, 1 mM EDTA, pH=7.5). A concentration of biotinylated aptamer solution is added to magnetic beads suspension, ensuring that the beads would be well functionalized with the aptamer, and the mixture are incubated with gentle mixing for a period of time (e.g., one hour). The biotinylated aptamers are attached to the streptavidin-coated beads via the biotin-streptavidin bond.

1. (canceled)

2. (canceled)

3. (canceled)

4. A method for detecting the absence, presence and/or quantity of a target allergen in a test sample comprising:
a) obtaining a test sample which is suspected to contain the target allergen,
b) placing the test sample into a sample analysis cartridge, wherein the cartridge comprises an input tunnel configured for receiving the test sample, a plurality of reservoirs which separately store sample preparation reagents and a substrate, and an analysis channel,
c) mixing the test sample with the sample preparation reagents stored in the reservoirs sequentially from the first reservoir, the second reservoir and the third reservoir, and so on, wherein the target allergen is hybridized with the preparation reagents,
d) initiating a testing protocol in a specialized computer which is configured to detect the sample analysis cartridge,
e) releasing the contents of the plurality of reservoirs into the analysis channel of the sample analysis cartridge, wherein one or more sensors are disposed on the analysis channel to detect the hybridized target allergen, and
f) processing and analyzing the detection signals to identify the absence, presence, and/or the quantity of the target allergen in the test sample.

5. The method of claim 4, wherein the test sample is obtained using a sample collection device which can be inserted into the input tunnel of the sample analysis cartridge and optionally, is pre-processed by cutting into small pieces, crushing into powder, smashing into small particles, heating, or a combination thereof.

6. (canceled)

7. The method of claim 4, wherein the sample preparation reagents stored in the first reservoir of the plurality of reservoirs include a plurality of magnetic particles, each magnetic particle having surface bound affinity molecules, a plurality of detector agents, and other reagents that facilitate the binding of the target allergen to the sample preparation reagents,
wherein the detector agents are provided, and a resulting hybridized mixture comprises a plurality of sandwich complexes formed of the target allergen from the test sample bound to both the surface affinity molecules on the surfaces of magnetic particles and detector agents.

8. The method of claim 4, wherein the sample preparation reagents stored in the first reservoir of the plurality of reservoirs include a plurality of magnetic particles, each magnetic particle having surface bound affinity molecules, a plurality of competitive binding agents, and other reagents that facilitate the binding of the target allergen to the sample preparation reagents,
wherein the competitive binding agents are provided, and a resulting hybridized mixture comprises a plurality of molecule complexes formed of the competitive binding agent bound to the surface affinity molecules on the surfaces of magnetic particles, and the target allergen from the test sample bound to the surface affinity molecules on the surfaces of magnetic particles, and wherein the amount of the competitive binding agents that bind to the magnetic particles is inversely proportional to the amount of the unbound target allergen in the test sample.

9. (canceled)

10. The method of claim 7, wherein the surface affinity molecules and the detector agents comprise aptamer derived signaling polynucleotides which bind the target allergen with high specificity and affinity.

11. (canceled)

12. The method of claim 8, wherein the surface affinity molecules and competitive binding agents comprise aptamer derived signaling polynucleotides which bind the target allergen with high specificity and affinity.

13. The method of claim 10, wherein the aptamer derived signaling polynucleotides comprise a core nucleic acid sequence selected from the group consisting of SEQ ID NO.: 6, SEQ ID NO.: 13, SEQ ID NO.: 27, SEQ ID NO.: 35, SEQ ID NO.: 44, SEQ ID NO.: 57, SEQ ID NO.: 61, SEQ ID NO.: 65, SEQ ID NO.: 71, SEQ ID NO.: 78, SEQ ID NO.: 84, SEQ ID NO.: 98, SEQ ID NO.: 110, SEQ ID NO.: 119, SEQ ID NO.: 126; SEQ ID NO.: 132, SEQ ID NO.: 137, SEQ ID NO.: 150, SEQ ID NO.: 158, SEQ ID NO.: 168, SEQ ID NO.: 178, SEQ ID NO.: 190, SEQ ID NO.: 194, SEQ ID NO.: 204, SEQ ID NO.: 215, SEQ ID NO.: 220, SEQ ID NO.: 228, SEQ ID NO.: 237, SEQ ID NO.: 247, SEQ ID NO.: 251, SEQ ID NO.: 255, SEQ ID NO.: 259, SEQ ID NO.: 263, SEQ ID NO.: 267, SEQ ID NO.: 271, SEQ ID NO.: 279, SEQ ID NO.: 296, SEQ ID NO.: 308, SEQ ID NO.: 316, SEQ ID NO.: 332, SEQ ID NO.: 334, SEQ ID NO.: 336, SEQ ID NO.: 338 and SEQ ID Nos.: 341-353.

14. The method of claim 13, wherein the signaling polynucleotides comprise the nucleic acid sequences selected from the group consisting of SEQ ID Nos.: 1-353.

15. The method of claim 14, wherein the nucleic acid sequence of the signaling polynucleotide is conjugated to the surface of a magnetic particle through a streptavidin and biotin interaction.

16. The method of claim 15, wherein the detector agent further comprises a signaling agent.

17. The method of claim 16, wherein the signaling agent is a peroxidase.

18. The method of claim 17, wherein the second reservoir of the plurality of the reservoirs within the sample analysis cartridge includes a substrate, wherein the substrate is introduced to the resulting hybridized mixture and induces a detection reaction.

19. The method of claim 18, wherein a third reservoir of the plurality reservoirs comprises wash solution.

20. The method of claim 19, wherein the resulting hybridized mixture flows out of the plurality of reservoirs into the analysis channel within the cartridge, wherein the magnetic particles within the mixture localize to a portion of the analysis channel, forming a localized sample, wherein one or more sensors are deposed on the portion of the analysis channel.

21. The method of claim 20, wherein the mixture flows into the analysis channel via capillary action.

22. The method of claim 21, further comprising a step of which the wash solution flows out of the reservoir into the analysis channel, wherein the wash solution removes, from the localized sample, detector agents or competitive binding agents that are not directly bound to magnetic particles.

23. The method of claim 20, wherein the one or more sensors are gold or other conducting metals which can detect an electrochemical reaction and wherein the one or more sensors are connected to a reader device which processes the detected signaling from the sensors and indicates the absence, presence and/or quantity of the target allergen in the test sample.

24. (canceled)

25. The method of claim 23, wherein the reader device further comprises a magnetic field generator and a circuit having a cartridge detection unit, wherein the reader device is coupled to the sample analysis cartridge, the magnetic field generated by the magnetic field generator is aligned with the sensor within the analysis channel of the sample analysis cartridge, and the circuit is electrically coupled to the sensors within the analysis channel.

26. A method of detecting the absence, presence and/or quantity of a target allergen in a test sample comprising:
a) obtaining a test sample which is suspected to contain the target allergen,
b) filtering the test sample using a filter configured to filter the test sample resulting in a filtrate comprising the target allergen,
c) delivering the filtrate of step (b) through a capillary to a surface of an integrated circuit which includes one or more sensor areas on the surface of said integrated circuit, wherein dried magnetic particles whose surfaces are chemically functionalized to react with the target allergen in the filtrate are pre-stored in the capillary channel or the sensor areas on the surface of the integrated circuit, and wherein the filtrate flows in the capillary channel and the target allergen in the filtrate binds the functionalized magnetic particles to form target magnetic particle complexes which can bind specifically onto the sensor areas on the surface of the integrated circuit,
d) detecting magnetic particles specifically bound to said one or more sensor areas using a plurality of sensors, and
e) transmitting signals detected in step (d) into indicative of the absence, presence and/or quantity of the target allergen in the test sample.

27. The method of claim 26 further comprising a step of manipulating the magnetic particles bound to said one or more sensor areas on the surface of the integrated circuit before detecting magnetic particles specifically bound to said one or more sensor areas, wherein the magnetic particles are manipulated by one or more magnetic field generators.

28. The method of claim 27, wherein the one or more magnetic field generators comprise one or more magnetic concentration field generators which generate one or more concentration fields to pull the magnetic particles to one or more sensor areas on the surface of the integrated circuit, and one or more magnetic separation field generators which generate one or more separation fields to remove the non-specifically bound magnetic particles from one or more sensor areas on the surface of the integrated circuit.

29. The method of claim 28, wherein one or more magnetic separation field generators are embedded in the integrated circuit.

30. The method of claim 26, wherein the chemically functionalized magnetic particles are conjugated to one or more aptamer derived signaling polynucleotides.

31. The method of claim 30, wherein the signaling polynucleotide comprises a core nucleic acid sequence selected from the group consisting of SEQ ID NO.: 6, SEQ ID NO.: 13, SEQ ID NO.: 27, SEQ ID NO.: 35, SEQ ID NO.: 44, SEQ ID NO.: 57, SEQ ID NO.: 61, SEQ ID NO.: 65, SEQ ID NO.: 71, SEQ ID NO.: 78, SEQ ID NO.: 84, SEQ ID NO.: 98, SEQ ID NO.: 110, SEQ ID NO.: 119, SEQ ID NO.: 126; SEQ ID NO.: 132, SEQ ID NO.: 137, SEQ ID NO.: 150, SEQ ID NO.: 158, SEQ ID NO.: 168, SEQ ID NO.: 178, SEQ ID NO.: 190, SEQ ID NO.: 194, SEQ ID NO.: 204, SEQ ID NO.: 215, SEQ ID NO.: 220, SEQ ID NO.: 228, SEQ ID NO.: 237, SEQ ID NO.: 247, SEQ ID NO.: 251, SEQ ID NO.: 255, SEQ ID NO.: 259, SEQ ID NO.: 263, SEQ ID NO.: 267, SEQ ID NO.: 271, SEQ ID NO.: 279, SEQ ID NO.: 296, SEQ ID NO.: 308, SEQ ID NO.: 316, SEQ ID NO.: 332, SEQ ID NO.: 334, SEQ ID NO.: 336, SEQ ID NO.: 338 and SEQ ID Nos.: 341-353.

32. The method of claim 31, wherein the signaling polynucleotides comprise the nucleic acid sequences selected from the group consisting of SEQ ID NOs.: 1-353.

33. The method of claim 32, wherein the magnetic particles are lyophilized into one or more microspheres.

34. The method of claim 27, wherein the sensors are a plurality of magnetic particle sensors which are embedded in the integrated circuit, wherein the magnetic particle sensors are capable of detecting magnetic particles specifically bound to the one or more sensor areas on the surface of the integrated circuit.

35. The method of claim 34, wherein the number of magnetic particles specifically bound to the one or more sensor areas on the surface of the integrated circuit is representative of the concentration of the target allergen in the sample presented on the filter.

36. The method of claim 27, wherein the sensors are a plurality of optical sensors which are embedded in the integrated circuit.

37. (canceled)

38. The method of claim 12, wherein the aptamer derived signaling polynucleotides comprise a core nucleic acid sequence selected from the group consisting of SEQ ID NO.: 6, SEQ ID NO.: 13, SEQ ID NO.: 27, SEQ ID NO.: 35, SEQ ID NO.: 44, SEQ ID NO.: 57, SEQ ID NO.: 61, SEQ ID NO.: 65, SEQ ID NO.: 71, SEQ ID NO.: 78, SEQ ID NO.: 84, SEQ ID NO.: 98, SEQ ID NO.: 110, SEQ ID NO.: 119, SEQ ID NO.: 126; SEQ ID NO.: 132, SEQ ID NO.: 137, SEQ ID NO.: 150, SEQ ID NO.: 158, SEQ ID NO.: 168, SEQ ID NO.: 178, SEQ ID NO.: 190, SEQ ID NO.: 194, SEQ ID NO.: 204, SEQ ID NO.: 215, SEQ ID NO.: 220, SEQ ID NO.: 228, SEQ ID NO.: 237, SEQ ID NO.: 247, SEQ ID NO.: 251, SEQ ID NO.: 255, SEQ ID NO.: 259, SEQ ID NO.: 263, SEQ ID NO.: 267, SEQ ID NO.: 271, SEQ ID NO.: 279, SEQ ID NO.: 296, SEQ ID NO.: 308, SEQ ID NO.: 316, SEQ ID NO.: 332, SEQ ID NO.: 334, SEQ ID NO.: 336, SEQ ID NO.: 338 and SEQ ID Nos.: 341-353.

39. The method of claim 38, wherein the signaling polynucleotides comprise the nucleic acid sequences selected from the group consisting of SEQ ID Nos.: 1-353.
